(19) United States
(12) Patent Application Publication
(10) Pub. No.: US 2026/0207872 A1
(43) Pub. Date: Jul. 23, 2026

(54) DEVICES, SYSTEMS, AND METHODS FOR DETECTION OF MEDICAL DEVICE

(71) Applicant: ResMed Pty Ltd, Bella Vista, New South Wales (AU)

(72) Inventors: David Noel CREUSOT, Sydney (AU); Michael James DENT, Sydney (AU); Samuel Thomas HORLER, Sydney (AU); Ting Lee TEH, Sydney (AU); Rudi VOON, Sydney (AU)

(21) Appl. No.: 19/134,996

(22) PCT Filed: Dec. 13, 2023
(86) PCT No.: PCT/AU2023/051296

§ 371 Date: Jun. 2, 2025

Related U.S. Application Data
(60) Provisional application No. 63/387,325, filed on Dec. 14, 2022.
(60) Provisional application No. 63/487,311, filed on Feb. 28, 2023.
(60) Provisional application No. 63/487,317, filed on Feb. 28, 2023.
(60) Provisional application No. 63/515,681, filed on Jul. 26, 2023.

(51) Int. Cl. A61M 16/08; A61M 16/06

(52) U.S. Cl. CPC A61M 16/0816; A61M 16/0683; A61M 16/0883; A61M 2205/14; A61M 2205/3592; A61M 2205/6027

(57) ABSTRACT

A conduit headgear may comprise an opening removably coupleable with an air circuit; a first portion extending from the opening to a first proximal end configured to couple with a patient interface; a second portion extending from the opening to a second proximal end and configured to couple with the patient interface; a first stretchable concertina portion extending along the first portion; a second stretchable concertina portion extending along the second portion; a first antenna located at a region adjacent the opening; a second antenna spaced apart from the first antenna; and a conductive electrical connection coupling the first antenna and the second antenna, wherein the electrical connection extends along at least a portion of either the first stretchable concertina portion or the second stretchable concertina portion, and where the electrical connection is configured to accommodate stretching of the first stretchable concertina portion or the second stretchable concertina portion.

### 1 CROSS-REFERENCE TO RELATED APPLICATIONS

This application claims priority to U.S. Provisional Application Nos. 63/387,325, filed Dec. 14, 2022, 63/487,311, filed Feb. 28, 2023, 63/487,317, filed Feb. 28, 2023, and 63/515,681, filed Jul. 26, 2023, each of which is incorporated herein by reference in its entirety.

### 2 BACKGROUND OF THE TECHNOLOGY

### 2.1 Field of the Technology

The present technology relates to one or more of the screening, diagnosis, monitoring, treatment, prevention and amelioration of respiratory-related disorders. The present technology also relates to medical devices, or apparatus, systems, and their use.

### 2.2 Description of the Related Art

### 2.2.1 Human Respiratory System and its Disorders

The respiratory system of the body facilitates gas exchange. The nose and mouth form the entrance to the airways of a patient.

The airways include a series of branching tubes, which become narrower, shorter and more numerous as they penetrate deeper into the lung. The prime function of the lung is gas exchange, allowing oxygen to move from the inhaled air into the venous blood and carbon dioxide to move in the opposite direction. The trachea divides into right and left main bronchi, which further divide eventually into terminal bronchioles. The bronchi make up the conducting airways, and do not take part in gas exchange. Further divisions of the airways lead to the respiratory bronchioles, and eventually to the alveoli. The alveolated region of the lung is where the gas exchange takes place, and is referred to as the respiratory zone. See “Respiratory Physiology”, by John B. West, Lippincott Williams & Wilkins, 9^{th} edition published 2012.

A range of respiratory disorders exist. Certain disorders may be characterised by particular events, e.g. apneas, hypopneas, and hyperpneas.

Examples of respiratory disorders include Obstructive Sleep Apnea (OSA), Cheyne-Stokes Respiration (CSR), respiratory insufficiency, Obesity Hyperventilation Syndrome (OHS), Chronic Obstructive Pulmonary Disease (COPD), Neuromuscular Disease (NMD) and Chest wall disorders.

Obstructive Sleep Apnea (OSA), a form of Sleep Disordered Breathing (SDB), is characterised by events including occlusion or obstruction of the upper air passage during sleep. It results from a combination of an abnormally small upper airway and the normal loss of muscle tone in the region of the tongue, soft palate and posterior oropharyngeal wall during sleep. The condition causes the affected patient to stop breathing for periods typically of 30 to 120 seconds in duration, sometimes 200 to 300 times per night. It often causes excessive daytime somnolence, and it may cause cardiovascular disease and brain damage. The syndrome is a common disorder, particularly in middle aged overweight males, although a person affected may have no awareness of the problem. See U.S. Pat. No. 4,944,310 (Sullivan).

Cheyne-Stokes Respiration (CSR) is another form of sleep disordered breathing. CSR is a disorder of a patient's respiratory controller in which there are rhythmic alternating periods of waxing and waning ventilation known as CSR cycles. CSR is characterised by repetitive de-oxygenation and re-oxygenation of the arterial blood. It is possible that CSR is harmful because of the repetitive hypoxia. In some patients CSR is associated with repetitive arousal from sleep, which causes severe sleep disruption, increased sympathetic activity, and increased afterload. See U.S. Pat. No. 6,532,959 (Berthon-Jones).

Respiratory failure is an umbrella term for respiratory disorders in which the lungs are unable to inspire sufficient oxygen or exhale sufficient CO2 to meet the patient's needs. Respiratory failure may encompass some or all of the following disorders.

A patient with respiratory insufficiency (a form of respiratory failure) may experience abnormal shortness of breath on exercise.

Obesity Hyperventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common. These include increased resistance to air movement, extended expiratory phase of respiration, and loss of the normal elasticity of the lung. Examples of COPD are emphysema and chronic bronchitis. COPD is caused by chronic tobacco smoking (primary risk factor), occupational exposures, air pollution and genetic factors. Symptoms include: dyspnea on exertion, chronic cough and sputum production.

Neuromuscular Disease (NMD) is a broad term that encompasses many diseases and ailments that impair the functioning of the muscles either directly via intrinsic muscle pathology, or indirectly via nerve pathology. Some NMD patients are characterised by progressive muscular impairment leading to loss of ambulation, being wheelchair-bound, swallowing difficulties, respiratory muscle weakness and, eventually, death from respiratory failure. Neuromuscular disorders can be divided into rapidly progressive and slowly progressive: (i) Rapidly progressive disorders: Characterised by muscle impairment that worsens over months and results in death within a few years (e.g. Amyotrophic lateral sclerosis (ALS) and Duchenne muscular dystrophy (DMD) in teenagers); (ii) Variable or slowly progressive disorders: Characterised by muscle impairment that worsens over years and only mildly reduces life expectancy (e.g. Limb girdle, Facioscapulohumeral and Myotonic muscular dystrophy). Symptoms of respiratory failure in NMD include: increasing generalised weakness, dysphagia, dyspnea on exertion and at rest, fatigue, sleepiness, morning headache, and difficulties with concentration and mood changes.

Chest wall disorders are a group of thoracic deformities that result in inefficient coupling between the respiratory muscles and the thoracic cage. The disorders are usually characterised by a restrictive defect and share the potential of long term hypercapnic respiratory failure. Scoliosis and/or kyphoscoliosis may cause severe respiratory failure. Symptoms of respiratory failure include: dyspnea on exertion, peripheral oedema, orthopnea, repeated chest infections, morning headaches, fatigue, poor sleep quality and loss of appetite.

A range of therapies have been used to treat or ameliorate such conditions. Furthermore, otherwise healthy individuals may take advantage of such therapies to prevent respiratory disorders from arising. However, these have a number of shortcomings.

### 2.2.2 Therapies

Various respiratory therapies, such as Continuous Positive Airway Pressure (CPAP) therapy, Non-invasive ventilation (NIV), Invasive ventilation (IV), and High Flow Therapy (HFT) have been used to treat one or more of the above respiratory disorders.

### 2.2.2.1 Respiratory Pressure Therapies

Respiratory pressure therapy is the application of a supply of air to an entrance to the airways at a controlled target pressure that is nominally positive with respect to atmosphere throughout the patient's breathing cycle (in contrast to negative pressure therapies such as the tank ventilator or cuirass).

Continuous Positive Airway Pressure (CPAP) therapy has been used to treat Obstructive Sleep Apnea (OSA). The mechanism of action is that continuous positive airway pressure acts as a pneumatic splint and may prevent upper airway occlusion, such as by pushing the soft palate and tongue forward and away from the posterior oropharyngeal wall. Treatment of OSA by CPAP therapy may be voluntary, and hence patients may elect not to comply with therapy if they find devices used to provide such therapy one or more of: uncomfortable, difficult to use, expensive and aesthetically unappealing.

Non-invasive ventilation (NIV) provides ventilatory support to a patient through the upper airways to assist the patient breathing and/or maintain adequate oxygen levels in the body by doing some or all of the work of breathing. The ventilatory support is provided via a non-invasive patient interface. NIV has been used to treat CSR and respiratory failure, in forms such as OHS, COPD, NMD and Chest Wall disorders. In some forms, the comfort and effectiveness of these therapies may be improved.

Invasive ventilation (IV) provides ventilatory support to patients that are no longer able to effectively breathe themselves and may be provided using a tracheostomy tube or endotracheal tube. In some forms, the comfort and effectiveness of these therapies may be improved.

### 2.2.2.2 Flow Therapies

Not all respiratory therapies aim to deliver a prescribed therapeutic pressure. Some respiratory therapies aim to deliver a prescribed respiratory volume, by delivering an inspiratory flow rate profile over a targeted duration, possibly superimposed on a positive baseline pressure. In other cases, the interface to the patient's airways is ‘open’ (unsealed) and the respiratory therapy may only supplement the patient's own spontaneous breathing with a flow of conditioned or enriched gas. In one example, High Flow therapy (HFT) is the provision of a continuous, heated, humidified flow of air to an entrance to the airway through an unsealed or open patient interface at a “treatment flow rate” that may be held approximately constant throughout the respiratory cycle. The treatment flow rate is nominally set to exceed the patient's peak inspiratory flow rate. HFT has been used to treat OSA, CSR, respiratory failure, COPD, and other respiratory disorders. One mechanism of action is that the high flow rate of air at the airway entrance improves ventilation efficiency by flushing, or washing out, expired CO2 from the patient's anatomical deadspace. Hence, HFT is thus sometimes referred to as a deadspace therapy (DST). Other benefits may include the elevated warmth and humidification (possibly of benefit in secretion management) and the potential for modest elevation of airway pressures. As an alternative to constant flow rate, the treatment flow rate may follow a profile that varies over the respiratory cycle.

Another form of flow therapy is long-term oxygen therapy (LTOT) or supplemental oxygen therapy. Doctors may prescribe a continuous flow of oxygen enriched air at a specified oxygen concentration (from 21%, the oxygen fraction in ambient air, to 100%) at a specified flow rate (e.g., 1 litre per minute (LPM), 2 LPM, 3 LPM, etc.) to be delivered to the patient's airway.

### 2.2.2.3 Supplementary Oxygen

For certain patients, oxygen therapy may be combined with a respiratory pressure therapy or HFT by adding supplementary oxygen to the pressurised flow of air. When oxygen is added to respiratory pressure therapy, this is referred to as RPT with in supplementary oxygen. When oxygen is added to HFT, the resulting therapy is referred to as HFT with supplementary oxygen.

### 2.2.3 Respiratory Therapy Systems

These respiratory therapies may be provided by a respiratory therapy system or device. Such systems and devices may also be used to screen, diagnose, or monitor a condition without treating it.

A respiratory therapy system may comprise a Respiratory Pressure Therapy Device (RPT device), an air circuit, a humidifier, a patient interface, an oxygen source, and data management.

Another form of therapy system is a mandibular repositioning device.

### 2.2.3.1 Patient Interface

A patient interface may be used to interface respiratory equipment to its wearer, for example by providing a flow of air to an entrance to the airways. The flow of air may be provided via a mask to the nose and/or mouth, a tube to the mouth or a tracheostomy tube to the trachea of a patient. Depending upon the therapy to be applied, the patient interface may form a seal, e.g., with a region of the patient's face, to facilitate the delivery of gas at a pressure at sufficient variance with ambient pressure to effect therapy, e.g., at a positive pressure of about 10 cmH2O relative to ambient pressure. For other forms of therapy, such as the delivery of oxygen, the patient interface may not include a seal sufficient to facilitate delivery to the airways of a supply of gas at a positive pressure of about 10 cmH2O. For flow therapies such as nasal HFT, the patient interface is configured to insufflate the nares but specifically to avoid a complete seal. One example of such a patient interface is a nasal cannula.

Certain other mask systems may be functionally unsuitable for the present field. For example, purely ornamental masks may be unable to maintain a suitable pressure. Mask systems used for underwater swimming or diving may be configured to guard against ingress of water from an external higher pressure, but not to maintain air internally at a higher pressure than ambient.

Certain masks may be clinically unfavourable for the present technology e.g. if they block airflow via the nose and only allow it via the mouth.

Certain masks may be uncomfortable or impractical for the present technology if they require a patient to insert a portion of a mask structure in their mouth to create and maintain a seal via their lips.

Certain masks may be impractical for use while sleeping, e.g. for sleeping while lying on one's side in bed with a head on a pillow.

The design of a patient interface presents a number of challenges. The face has a complex three-dimensional shape. The size and shape of noses and heads varies considerably between individuals. Since the head includes bone, cartilage and soft tissue, different regions of the face respond differently to mechanical forces. The jaw or mandible may move relative to other bones of the skull. The whole head may move during the course of a period of respiratory therapy.

As a consequence of these challenges, some masks suffer from being one or more of obtrusive, aesthetically undesirable, costly, poorly fitting, difficult to use, and uncomfortable especially when worn for long periods of time or when a patient is unfamiliar with a system. Wrongly sized masks can give rise to reduced compliance, reduced comfort and poorer patient outcomes. Masks designed solely for aviators, masks designed as part of personal protection equipment (e.g. filter masks), SCUBA masks, or for the administration of anaesthetics may be tolerable for their original application, but nevertheless such masks may be undesirably uncomfortable to be worn for extended periods of time, e.g., several hours. This discomfort may lead to a reduction in patient compliance with therapy. This is even more so if the mask is to be worn during sleep.

CPAP therapy is highly effective to treat certain respiratory disorders, provided patients comply with therapy. If a mask is uncomfortable, or difficult to use a patient may not comply with therapy. Since it is often recommended that a patient regularly wash their mask, if a mask is difficult to clean (e.g., difficult to assemble or disassemble), patients may not clean their mask and this may impact on patient compliance.

While a mask for other applications (e.g. aviators) may not be suitable for use in treating sleep disordered breathing, a mask designed for use in treating sleep disordered breathing may be suitable for other applications.

For these reasons, patient interfaces for delivery of CPAP during sleep form a distinct field.

### 2.2.3.1.1 Seal-Forming Structure

Patient interfaces may include a seal-forming structure. Since it is in direct contact with the patient's face, the shape and configuration of the seal-forming structure can have a direct impact the effectiveness and comfort of the patient interface.

A patient interface may be partly characterised according to the design intent of where the seal-forming structure is to engage with the face in use. In one form of patient interface, a seal-forming structure may comprise a first sub-portion to form a seal around the left naris and a second sub-portion to form a seal around the right naris. In one form of patient interface, a seal-forming structure may comprise a single element that surrounds both nares in use. Such single element may be designed to for example overlay an upper lip region and a nasal bridge region of a face. In one form of patient interface a seal-forming structure may comprise an element that surrounds a mouth region in use, e.g. by forming a seal on a lower lip region of a face. In one form of patient interface, a seal-forming structure may comprise a single element that surrounds both nares and a mouth region in use. These different types of patient interfaces may be known by a variety of names by their manufacturer including nasal masks, full-face masks, nasal pillows, nasal puffs and oro-nasal masks.

A seal-forming structure that may be effective in one region of a patient's face may be inappropriate in another region, e.g. because of the different shape, structure, variability and sensitivity regions of the patient's face. For example, a seal on swimming goggles that overlays a patient's forehead may not be appropriate to use on a patient's nose.

Certain seal-forming structures may be designed for mass manufacture such that one design fit and be comfortable and effective for a wide range of different face shapes and sizes. To the extent to which there is a mismatch between the shape of the patient's face, and the seal-forming structure of the mass-manufactured patient interface, one or both must adapt in order for a seal to form.

One type of seal-forming structure extends around the periphery of the patient interface, and is intended to seal against the patient's face when force is applied to the patient interface with the seal-forming structure in confronting engagement with the patient's face. The seal-forming structure may include an air or fluid filled cushion, or a moulded or formed surface of a resilient seal element made of an elastomer such as a rubber. With this type of seal-forming structure, if the fit is not adequate, there will be gaps between the seal-forming structure and the face, and additional force will be required to force the patient interface against the face in order to achieve a seal.

Another type of seal-forming structure incorporates a flap seal of thin material positioned about the periphery of the mask so as to provide a self-sealing action against the face of the patient when positive pressure is applied within the mask. Like the previous style of seal forming portion, if the match between the face and the mask is not good, additional force may be required to achieve a seal, or the mask may leak. Furthermore, if the shape of the seal-forming structure does not match that of the patient, it may crease or buckle in use, giving rise to leaks.

Another type of seal-forming structure may comprise a friction-fit element, e.g. for insertion into a naris, however some patients find these uncomfortable.

Another form of seal-forming structure may use adhesive to achieve a seal. Some patients may find it inconvenient to constantly apply and remove an adhesive to their face.

A range of patient interface seal-forming structure technologies are disclosed in the following patent applications, assigned to ResMed Limited: WO 1998/004,310; WO 2006/074,513; WO 2010/135,785.

One form of nasal pillow is found in the Adam Circuit manufactured by Puritan Bennett. Another nasal pillow, or nasal puff is the subject of U.S. Pat. No. 4,782,832 (Trimble et al.), assigned to Puritan-Bennett Corporation.

ResMed Limited has manufactured the following products that incorporate nasal pillows: SWIFT™ nasal pillows mask, SWIFT™ II nasal pillows mask, SWIFT™ LT nasal pillows mask, SWIFT™ FX nasal pillows mask and MIRAGE LIBERTY™ full-face mask. The following patent applications, assigned to ResMed Limited, describe examples of nasal pillows masks: International Patent Application WO2004/073,778 (describing amongst other things aspects of the ResMed Limited SWIFT™ nasal pillows), US Patent Application 2009/0044808 (describing amongst other things aspects of the ResMed Limited SWIFT™ LT nasal pillows); International Patent Applications WO 2005/063,328 and WO 2006/130,903 (describing amongst other things aspects of the ResMed Limited MIRAGE LIBERTY™ full-face mask); International Patent Application WO 2009/052,560 (describing amongst other things aspects of the ResMed Limited SWIFT™ FX nasal pillows).

### 2.2.3.1.2 Positioning and Stabilising

A seal-forming structure of a patient interface used for positive air pressure therapy is subject to the corresponding force of the air pressure to disrupt a seal. Thus, a variety of techniques have been used to position the seal-forming structure, and to maintain it in sealing relation with the appropriate portion of the face.

One technique is the use of adhesives. See for example US Patent Application Publication No. US 2010/0000534. However, the use of adhesives may be uncomfortable for some.

Another technique is the use of one or more straps and/or stabilising harnesses. Many such harnesses suffer from being one or more of ill-fitting, bulky, uncomfortable and awkward to use.

### 2.2.3.2 Respiratory Pressure Therapy (RPT) Device

A respiratory pressure therapy (RPT) device may be used individually or as part of a system to deliver one or more of a number of therapies described above, such as by operating the device to generate a flow of air for delivery to an interface to the airways. The flow of air may be pressure-controlled (for respiratory pressure therapies) or flow-controlled (for flow therapies such as HFT). Thus, RPT devices may also act as flow therapy devices. Examples of RPT devices include a CPAP device and a ventilator.

Air pressure generators are known in a range of applications, e.g. industrial-scale ventilation systems. However, air pressure generators for medical applications have particular requirements not fulfilled by more generalised air pressure generators, such as the reliability, size and weight requirements of medical devices. In addition, even devices designed for medical treatment may suffer from shortcomings, pertaining to one or more of: comfort, noise, ease of use, efficacy, size, weight, manufacturability, cost, and reliability.

An example of the special requirements of certain RPT devices is acoustic noise.

Table of noise output levels of prior RPT devices (one specimen only, measured using test method specified in ISO 3744 in CPAP mode at 10 cmH2O).

| | A-weighted sound | Year |
|---|---|---|
| RPT Device name | pressure level dB(A) | (approx.) |
| C-Series TangoTM | 31.9 | 2007 |
| C-Series TangoTM with Humidifier | 33.1 | 2007 |
| S8 EscapeTM II | 30.5 | 2005 |
| S8 EscapeTM II with H4iTM | 31.1 | 2005 |
| Humidifier | | |
| S9 AutoSetTM | 26.5 | 2010 |
| S9 AutoSetTM with H5i Humidifier | 28.6 | 2010 |

One known RPT device used for treating sleep disordered breathing is the S9 Sleep Therapy System, manufactured by ResMed Limited. Another example of an RPT device is a ventilator. Ventilators such as the ResMed Stellar™ Series of Adult and Paediatric Ventilators may provide support for invasive and non-invasive non-dependent ventilation for a range of patients for treating a number of conditions such as but not limited to NMD, OHS and COPD.

The ResMed Elisée™ 150 ventilator and ResMed VS III™ ventilator may provide support for invasive and non-invasive dependent ventilation suitable for adult or paediatric patients for treating a number of conditions. These ventilators provide volumetric and barometric ventilation modes with a single or double limb circuit. RPT devices typically comprise a pressure generator, such as a motor-driven blower or a compressed gas reservoir, and are configured to supply a flow of air to the airway of a patient. In some cases, the flow of air may be supplied to the airway of the patient at positive pressure. The outlet of the RPT device is connected via an air circuit to a patient interface such as those described above.

The designer of a device may be presented with an infinite number of choices to make. Design criteria often conflict, meaning that certain design choices are far from routine or inevitable. Furthermore, the comfort and efficacy of certain aspects may be highly sensitive to small, subtle changes in one or more parameters.

### 2.2.3.3 Air Circuit

An air circuit is a conduit or a tube constructed and arranged to allow, in use, a flow of air to travel between two components of a respiratory therapy system such as the RPT device and the patient interface. In some cases, there may be separate limbs of the air circuit for inhalation and exhalation. In other cases, a single limb air circuit is used for both inhalation and exhalation.

### 2.2.3.4 Humidifier

Delivery of a flow of air without humidification may cause drying of airways. The use of a humidifier with an RPT device and the patient interface produces humidified gas that minimizes drying of the nasal mucosa and increases patient airway comfort. In addition, in cooler climates, warm air applied generally to the face area in and about the patient interface is more comfortable than cold air.

A range of artificial humidification devices and systems are known, however they may not fulfil the specialised requirements of a medical humidifier.

Medical humidifiers are used to increase humidity and/or temperature of the flow of air in relation to ambient air when required, typically where the patient may be asleep or resting (e.g. at a hospital). A medical humidifier for bedside placement may be small. A medical humidifier may be configured to only humidify and/or heat the flow of air delivered to the patient without humidifying and/or heating the patient's surroundings. Room-based systems (e.g. a sauna, an air conditioner, or an evaporative cooler), for example, may also humidify air that is breathed in by the patient, however those systems would also humidify and/or heat the entire room, which may cause discomfort to the occupants. Furthermore, medical humidifiers may have more stringent safety constraints than industrial humidifiers.

While a number of medical humidifiers are known, they can suffer from one or more shortcomings. Some medical humidifiers may provide inadequate humidification, some are difficult or inconvenient to use by patients.

### 2.2.3.5 Oxygen Source

Experts in this field have recognized that exercise for respiratory failure patients provides long term benefits that slow the progression of the disease, improve quality of life and extend patient longevity. Most stationary forms of exercise like tread mills and stationary bicycles, however, are too strenuous for these patients. As a result, the need for mobility has long been recognized. Until recently, this mobility has been facilitated by the use of small compressed oxygen tanks or cylinders mounted on a cart with dolly wheels. The disadvantage of these tanks is that they contain a finite amount of oxygen and are heavy, weighing about 50 pounds when mounted.

Oxygen concentrators have been in use for about 50 years to supply oxygen for respiratory therapy. Traditional oxygen concentrators have been bulky and heavy making ordinary ambulatory activities with them difficult and impractical. Recently, companies that manufacture large stationary oxygen concentrators began developing portable oxygen concentrators (POCs). The advantage of POCs is that they can produce a theoretically endless supply of oxygen. In order to make these devices small for mobility, the various systems necessary for the production of oxygen enriched gas are condensed. POCs seek to utilize their produced oxygen as efficiently as possible, in order to minimise weight, size, and power consumption. This may be achieved by delivering the oxygen as series of pulses, each pulse or “bolus” timed to coincide with the onset of inhalation. This therapy mode is known as pulsed oxygen delivery (POD) or demand mode, in contrast with traditional continuous flow delivery more suited to stationary oxygen concentrators.

### 2.2.3.6 Data Management

There may be clinical reasons to obtain data to determine whether the patient prescribed with respiratory therapy has been “compliant”, e.g. that the patient has used their RPT device according to one or more “compliance rules”. One example of a compliance rule for CPAP therapy is that a patient, in order to be deemed compliant, is required to use the RPT device for at least four hours a night for at least 21 of 30 consecutive days. In order to determine a patient's compliance, a provider of the RPT device, such as a health care provider, may manually obtain data describing the patient's therapy using the RPT device, calculate the usage over a predetermined time period, and compare with the compliance rule. Once the health care provider has determined that the patient has used their RPT device according to the compliance rule, the health care provider may notify a third party that the patient is compliant.

There may be other aspects of a patient's therapy that would benefit from communication of therapy data to a third party or external system.

Existing processes to communicate and manage such data can be one or more of costly, time-consuming, and error-prone.

### 2.2.3.7 Vent Technologies

Some forms of treatment systems may include a vent to allow the washout of exhaled carbon dioxide. The vent may allow a flow of gas from an interior space of a patient interface, e.g., the plenum chamber, to an exterior of the patient interface, e.g., to ambient.

The vent may comprise an orifice and gas may flow through the orifice in use of the mask. Many such vents are noisy. Others may become blocked in use and thus provide insufficient washout. Some vents may be disruptive of the sleep of a bed partner 1100 of the patient 1000, e.g. through noise or focussed airflow.

ResMed Limited has developed a number of improved mask vent technologies. See International Patent Application Publication No. WO 1998/034,665; International Patent Application Publication No. WO 2000/078,381; U.S. Pat. No. 6,581,594; US Patent Application Publication No. US 2009/0050156; US Patent Application Publication No. 2009/0044808.

Table of noise of prior masks (ISO 17510-2:2007, 10 cmH2O pressure at 1m)

| | | | A-weighted | |
|---|---|---|---|---|
| | | A-weighted | sound | |
| | | sound power | pressure | |
| | Mask | level dB(A) | dB(A) | Year |
| Mask name | type | (uncertainty) | (uncertainty) | (approx.) |
| Glue-on (*) | nasal | 50.9 | 42.9 | 1981 |
| ResCare standard (*) | nasal | 31.5 | 23.5 | 1993 |
| ResMed | nasal | 29.5 | 21.5 | 1998 |
| MirageTM (*) | | | | |
| ResMed | nasal | 36 (3) | 28 (3) | 2000 |
| UltraMirageTM | | | | |
| ResMed Mirage | nasal | 32 (3) | 24 (3) | 2002 |
| ActivaTM | | | | |
| ResMed Mirage | nasal | 30 (3) | 22 (3) | 2008 |
| MicroTM | | | | |
| ResMed MirageTM | nasal | 29 (3) | 22 (3) | 2008 |
| SoftGel | | | | |
| ResMed MirageTM | nasal | 26 (3) | 18 (3) | 2010 |
| FX | | | | |
| ResMed Mirage | nasal | 37 | 29 | 2004 |
| SwiftTM (*) | pillows | | | |
| ResMed Mirage | nasal | 28 (3) | 20 (3) | 2005 |
| SwiftTM II | pillows | | | |
| ResMed Mirage | nasal | 25 (3) | 17 (3) | 2008 |
| SwiftTM LT | pillows | | | |
| ResMed AirFit P10 | nasal | 21 (3) | 13 (3) | 2014 |
| | pillows | | | |

(*one specimen only, measured using test method specified in ISO 3744 in CPAP mode at 10 cmH2O)

Sound pressure values of a variety of objects are listed below:

| | A-weighted sound | |
|---|---|---|
| Object | pressure dB(A) | Notes |
| Vacuum cleaner: Nilfisk | 68 | ISO 3744 at 1 m |
| Walter Broadly Litter Hog: B+ | | distance |
| Grade | | |
| Conversational speech | 60 | 1 m distance |
| Average home | 50 | |
| Quiet library | 40 | |
| Quiet bedroom at night | 30 | |
| Background in TV studio | 20 | |

### 2.2.4 Screening, Diagnosis, and Monitoring Systems

Polysomnography (PSG) is a conventional system for diagnosis and monitoring of cardio-pulmonary disorders, and typically involves expert clinical staff to apply the system. PSG typically involves the placement of 15 to 20 contact sensors on a patient in order to record various bodily signals such as electroencephalography (EEG), electrocardiography (ECG), electrooculography (EOG), electromyography (EMG), etc. PSG for sleep disordered breathing has involved two nights of observation of a patient in a clinic, one night of pure diagnosis and a second night of titration of treatment parameters by a clinician. PSG is therefore expensive and inconvenient. In particular, it is unsuitable for home screening/diagnosis/monitoring of sleep disordered breathing.

Screening and diagnosis generally describe the identification of a condition from its signs and symptoms. Screening typically gives a true/false result indicating whether or not a patient's SDB is severe enough to warrant further investigation, while diagnosis may result in clinically actionable information. Screening and diagnosis tend to be one-off processes, whereas monitoring the progress of a condition can continue indefinitely. Some screening/diagnosis systems are suitable only for screening/diagnosis, whereas some may also be used for monitoring.

Clinical experts may be able to screen, diagnose, or monitor patients adequately based on visual observation of PSG signals. However, there are circumstances where a clinical expert may not be available, or a clinical expert may not be affordable. Different clinical experts may disagree on a patient's condition. In addition, a given clinical expert may apply a different standard at different times.

### 3 BRIEF SUMMARY OF THE TECHNOLOGY

The present technology is directed towards providing medical devices used in the screening, diagnosis, monitoring, amelioration, treatment, or prevention of respiratory disorders having one or more of improved comfort, cost, efficacy, ease of use and manufacturability.

A first aspect of the present technology relates to apparatus used in the screening, diagnosis, monitoring, amelioration, treatment or prevention of a respiratory disorder.

Another aspect of the present technology relates to methods used in the screening, diagnosis, monitoring, amelioration, treatment or prevention of a respiratory disorder.

An aspect of certain forms of the present technology is to provide methods and/or apparatus that improve the compliance of patients with respiratory therapy.

One form of the present technology comprises a positioning and stabilising structure configured to provide a force to hold the seal-forming structure in a therapeutically effective position on the patient's head. The positioning and stabilising structure includes at least one strap.

One form of the present technology comprises a patient interface comprising a plenum chamber, a seal-forming structure, and a positioning and stabilising structure.

One form of the present technology comprises patient interface comprising a plenum chamber pressurisable to a therapeutic pressure of at least 4 cmH2O above ambient air pressure. The plenum chamber includes at least one plenum chamber inlet port sized and structured to receive a flow of air at the therapeutic pressure for breathing by a patient. The patient interface also comprises a seal-forming structure that is constructed and arranged to form a seal with a region of the patient's face surrounding an entrance to the patient's airways. The seal-forming structure has a hole therein such that the flow of air at said therapeutic pressure is delivered to at least an entrance to the patient's nares. The seal-forming structure is constructed and arranged to maintain said therapeutic pressure in the plenum chamber throughout the patient's respiratory cycle in use. The patient interface also comprises a positioning and stabilising structure to provide a force to hold the seal-forming structure in a therapeutically effective position on the patient's head.

Another aspect of one form of the present technology is a series of modular elements that may be interconnected in order to form different styles of patient interfaces.

In one form, there are at least two versions or styles of each modular element. The versions or styles may be interchangeably used with one another in order to form different modular assemblies.

One form of the present technology comprises a conduit headgear, comprising: an opening configured to removably couple with an air circuit; a first portion extending from the opening to a first proximal end, wherein the first proximal end is configured to couple with a patient interface; a second portion extending from the opening to a second proximal end, wherein the second proximal end is configured to couple with the patient interface; a first stretchable concertina portion extending along a length of the first portion; a second stretchable concertina portion extending along a length of the second portion; a first antenna located at a region adjacent the opening; a second antenna spaced apart from the first antenna; and a conductive electrical connection coupling the first antenna and the second antenna, wherein the electrical connection extends along at least a portion of either the first stretchable concertina portion or the second stretchable concertina portion, and where the electrical connection is configured to accommodate stretching of the first stretchable concertina portion or the second stretchable concertina portion.

In an aspect of the present technology, the second antenna may be located at a region adjacent the first proximal end or the second proximal end.

In another aspect, the electrical connection has a serpentine or a zig-zag path at least where the electrical connection extends along the at least a portion of either the first stretchable concertina portion or the second stretchable concertina portion.

In a further aspect, the electrical connection may be formed of a wire or a stretchable material.

In some forms, the electrical connection may be located on an external surface of the first portion or the second portion.

In at least one form, the electrical connection may be located within a wall of the first portion or the second portion.

In aspects, the electrical connection may be incorporated as part of a sleeve covering an outer surface of at least one of the first portion or the second portion.

In an aspect of the present technology, the sleeve may be formed of a stretchable textile material.

In forms, the electrical connection may be in the form of a conductive trace, a conductive thread, or a metal gel.

In aspects of the present technology, the electrical connection may be printed, thermally bonded, or sewn on the sleeve.

In some aspects, the electrical connection may be a stretchable conductive silicone or thread printed or thermally bonded to an external surface of the first portion or the second portion.

In further aspects, the first antenna may encircle the opening.

In another form of the present technology, a conduit headgear may comprise: an opening configured to removably couple with an air circuit; a first portion extending from the opening to a first proximal end, wherein the first proximal end is configured to couple with a patient interface; a second portion extending from the opening to a second proximal end, wherein the second proximal end is configured to couple with the patient interface; a first stretchable concertina portion extending along a length of the first portion; a second stretchable concertina portion extending along a length of the second portion; a first antenna located at a region adjacent the opening; a second antenna spaced apart from the first antenna; and a sleeve covering at least one of the first portion or the second portion, wherein the sleeve comprises a conductive electrical connection extending along the sleeve and electrically coupling the first antenna and the second antenna.

In aspects of the present technology, the sleeve may be formed of a stretchable textile material.

In forms, the sleeve may be thermally bonded to an outer surface of at least one of the first portion or the second portion.

In further forms, at least one of the first antenna or the second antenna may be incorporated as part of the sleeve.

In other forms, the sleeve may be removably coupled to the first portion or the second portion.

In aspects, the sleeve may cover the first portion and the second portion.

In some forms, the second antenna may be located at a region adjacent the first proximal end or the second proximal end.

A further form of the present technology may comprise a conduit headgear, comprising: an opening configured to removably couple with an air circuit; a first portion extending from the opening to a first proximal end, wherein the first proximal end is configured to couple with a patient interface; a second portion extending from the opening to a second proximal end, wherein the second proximal end is configured to couple with the patient interface; a first stretchable concertina portion extending along a length of the first portion; a second stretchable concertina portion extending along a length of the second portion; a first antenna located at a region adjacent the opening; a second antenna spaced apart from the first antenna; and a stretchable conductive electrical connection electrically coupling the first antenna and the second antenna, wherein the electrical connection extends along at least a portion of either the first stretchable concertina portion or the second stretchable concertina portion.

In one form, the electrical connection may be formed of conductive silicone.

In further forms, the electrical connection may be in the form of a thread or wire.

In aspects, the electrical connection may be printed or thermally bonded on an external surface of the first portion or the second portion.

A further form of the present technology may comprise a conduit comprising a portion configured to extend from a distal end to a proximal end, a stretchable concertina portion configured to extend along a length of the portion, and a stretchable conductor configured to extend along at least a portion of the stretchable concertina portion. In an example, the distal end is configured to couple with an air circuit. In an example, the proximal end is configured to couple with a patient interface. In an example, the conduit further comprises a first antenna and a second antenna spaced apart from the first antenna, wherein the stretchable conductor is configured to electrically couple the first antenna and the second antenna.

The methods, systems, devices and apparatus described may be implemented to improve the functionality of a processor, such as a processor of a specific purpose computer, respiratory monitor and/or a respiratory therapy apparatus. Moreover, the described methods, systems, devices and apparatus can provide improvements in the technological field of automated management, monitoring and/or treatment of respiratory conditions, including, for example, sleep disordered breathing.

Of course, portions of the aspects may form sub-aspects of the present technology. Also, various ones of the sub-aspects and/or aspects may be combined in various manners and also constitute additional aspects or sub-aspects of the present technology.

Other features of the technology will be apparent from consideration of the information contained in the following detailed description, abstract, drawings and claims.

### 4 BRIEF DESCRIPTION OF THE DRAWINGS

The present technology is illustrated by way of example, and not by way of limitation, in the Figures of the accompanying drawings, in which like reference numerals refer to similar elements including:

FIGS. 1A-1C each illustrate various configurations of a respiratory therapy system in use.

FIG. 2 shows a patient interface in the form of a nasal mask in accordance with one form of the present technology.

FIG. 3A is a schematic diagram of the pneumatic path of an RPT device in accordance with one form of the present technology. The directions of upstream and downstream are indicated with reference to the blower and the patient interface. The blower is defined to be upstream of the patient interface and the patient interface is defined to be downstream of the blower, regardless of the actual flow direction at any particular moment. Items which are located within the pneumatic path between the blower and the patient interface are downstream of the blower and upstream of the patient interface.

FIG. 3B is a schematic diagram of the electrical components of an RPT device in accordance with one form of the present technology.

FIG. 3C is a schematic diagram of the algorithms implemented in an RPT device in accordance with one form of the present technology.

FIG. 4A shows a perspective view of a cushion of a patient interface configured to be worn by a patient and convey pressurized air to the patient's nose and the patient's mouth.

FIG. 4B shows a perspective view of a cushion of a patient interface configured to be worn by a patient and convey pressurized air to the patient's nose.

FIG. 4C shows a perspective view of tubes usable with either the cushion of FIG. 4A or the cushion of FIG. 4B.

FIG. 4D shows a perspective view of rigidiser arms usable with either the cushion of FIG. 4A of the cushion of FIG. 4B.

FIG. 4E shows a perspective view of headgear straps usable with the cushion of FIG. 4A.

FIG. 4F shows a perspective view of headgear straps usable with the cushion of FIG. 4B.

FIG. 4G shows a front view of a pair of sleeves that is removably fitted to either the tubes of FIG. 4C or the rigidiser arms of FIG. 4D.

FIG. 4H shows a front view of a full sleeve that is removably fitted to the rigidiser arms of FIG. 4D.

FIG. 4I shows a front perspective view of yet another alternate form of a full sleeve that is removably fitted to the rigidiser arms of FIG. 4D.

FIG. 4J is a front view of a patient wearing the cushion of FIG. 4A connected to the tubes of FIG. 4C, the headgear straps of FIG. 4E, and the sleeves of FIG. 4G.

FIG. 4K is a front view of a patient wearing the cushion of FIG. 4A connected to the rigidiser arms of FIG. 4D, the headgear straps of FIG. 4E, and the sleeve of FIG. 4H.

FIG. 4L is a front view of a patient wearing the cushion of FIG. 4B connected to the conduit headgear of FIG. 4C, and the headgear straps of FIG. 4F.

FIG. 4M is a front view of a patient wearing the cushion of FIG. 4B connected to the rigidiser arms of FIG. 4D, the headgear straps of FIG. 4F, and the sleeve of FIG. 4I.

FIG. 4N is an isolated perspective view of the vent of FIG. 4L.

FIG. 4O is an isolated perspective view of a portion of the air circuit of FIG. 4M.

FIG. 4P is a schematic view illustrating the possible combinations of the patient interfaces.

FIG. 5A illustrates a schematic view of a medical system, according to aspects of this disclosure.

FIG. 5B illustrates a schematic view of a medical system, according to aspects of this disclosure.

FIG. 6 illustrates a perspective view of an exemplary embodiment, according to aspects of this disclosure.

FIG. 7 illustrates perspective view of an exemplary embodiment, according to aspects of this disclosure.

FIG. 8 illustrates an exemplary configuration of antennas and tags in use, according to aspects of this disclosure.

### 5 DETAILED DESCRIPTION OF EXAMPLES OF THE TECHNOLOGY

Before the present technology is described in further detail, it is to be understood that the technology is not limited to the particular examples described herein, which may vary. It is also to be understood that the terminology used in this disclosure is for the purpose of describing only the particular examples discussed herein, and is not intended to be limiting.

The following description is provided in relation to various examples which may share one or more common characteristics and/or features. It is to be understood that one or more features of any one example may be combinable with one or more features of another example or other examples. In addition, any single feature or combination of features in any of the examples may constitute a further example.

Reference will now be made in detail to aspects of the present disclosure, examples of which are illustrated in the accompanying drawings. Wherever possible, the same or similar reference numbers will be used through the drawings to refer to the same or like parts. The term “distal” refers to a portion farthest away from a user (e.g., patient). By contrast, the term “proximal” refers to a portion closest to the user.

Both the foregoing general description and the following detailed description are exemplary and explanatory only and are not restrictive of the features, as claimed. As used herein, the terms “comprises,” “comprising,” “having,” “including,” or other variations thereof, are intended to cover a non-exclusive inclusion such that a process, method, article, or apparatus that comprises a list of elements does not include only those elements, but may include other elements not expressly listed or inherent to such a process, method, article, or apparatus. In this disclosure, relative terms, such as, for example, “about,” “substantially,” “generally,” and “approximately” are used to indicate a possible variation of ±10% in a stated value or characteristic.

### 5.1 Therapy

In one form, the present technology comprises a method for treating a respiratory disorder comprising applying positive pressure to the entrance of the airways of a patient 1000.

In certain examples of the present technology, a supply of air at positive pressure is provided to the nasal passages of the patient via one or both nares.

In certain examples of the present technology, mouth breathing is limited, restricted or prevented.

### 5.2 Respiratory Therapy Systems

In one form, the present technology comprises a respiratory therapy system for treating a respiratory disorder. The respiratory therapy system may comprise an RPT device 4000 for supplying a flow of air to the patient 1000 via an air circuit 4170 and a patient interface 3000 or 3800.

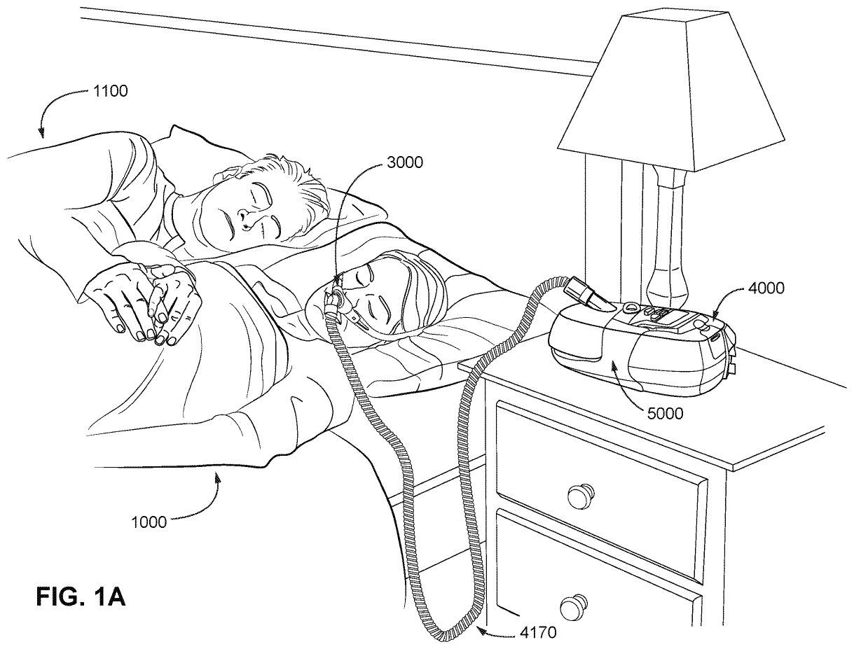

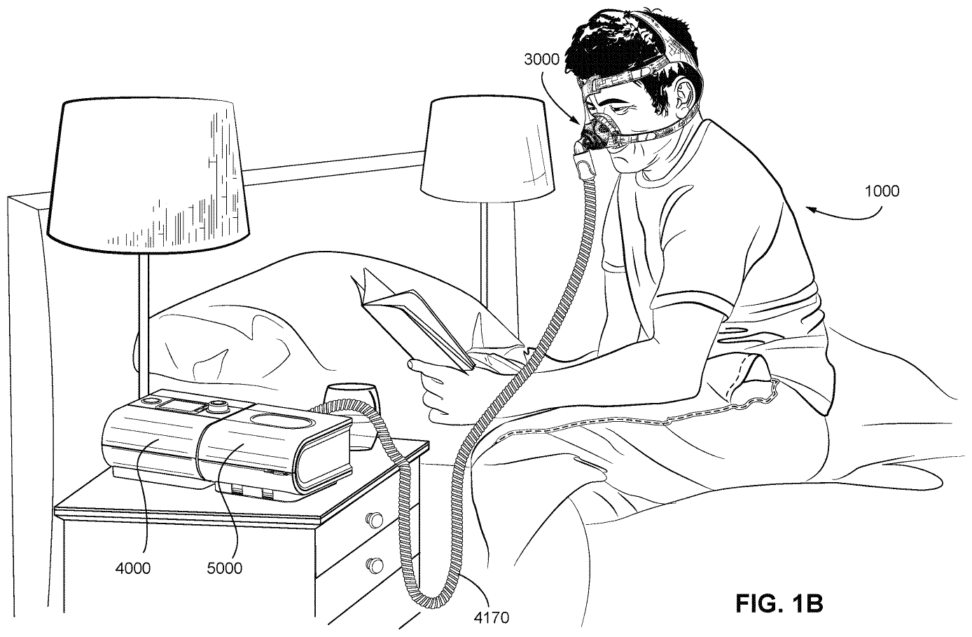

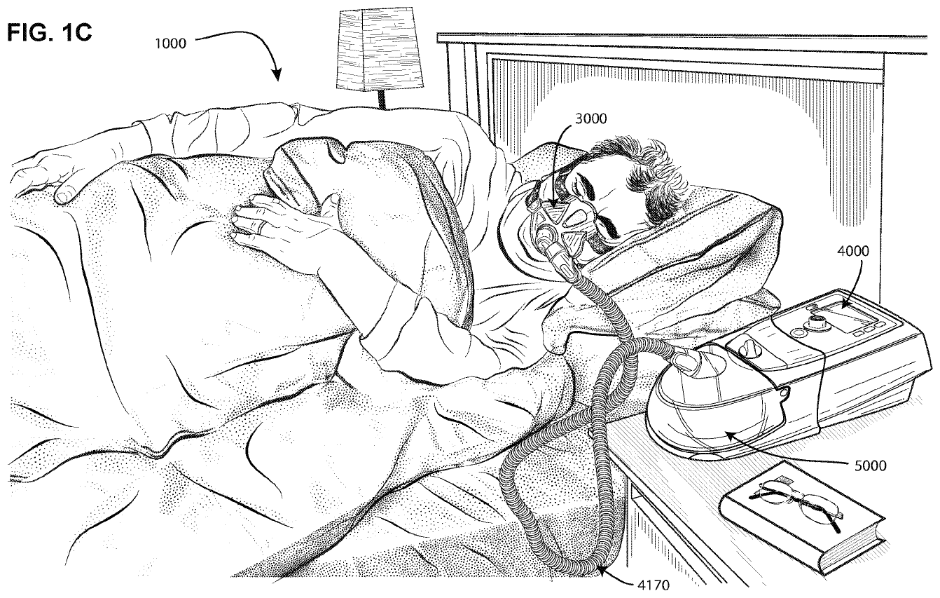

FIG. 1A shows a respiratory therapy system including a patient 1000 wearing a patient interface 3000, in the form of nasal pillows, receiving a supply of air at positive pressure from an RPT device 4000. Air from the RPT device 4000 is humidified in a humidifier 5000, and passes along an air circuit 4170 to the patient 1000. A bed partner 1100 is also shown. The patient is sleeping in a supine sleeping position.

FIG. 1B shows an alternative configuration of the respiratory therapy system including a patient 1000 wearing a patient interface 3000, in the form of a nasal mask, receiving a supply of air at positive pressure from an RPT device 4000. Air from the RPT device is humidified in a humidifier 5000, and passes along an air circuit 4170 to the patient 1000.

FIG. 1C shows a further alternative configuration of the respiratory therapy system including a patient 1000 wearing a patient interface 3000, in the form of a full-face mask, receiving a supply of air at positive pressure from an RPT device 4000. Air from the RPT device is humidified in a humidifier 5000, and passes along an air circuit 4170 to the patient 1000. The patient is sleeping in a side sleeping position.

An RPT device 4000 in accordance with one aspect of the present technology comprises mechanical, pneumatic, and/or electrical components and is configured to execute one or more algorithms 4300, such as any of the methods, in whole or in part, described herein. The RPT device 4000 may be configured to generate a flow of air for delivery to a patient's airways, such as to treat one or more of the respiratory conditions described elsewhere in the present document.

In one form, the RPT device 4000 is constructed and arranged to be capable of delivering a flow of air in a range of −20 L/min to +150 L/min while maintaining a positive pressure of at least 4 cmH2O, or at least 10 cmH2O, or at least 20 cmH2O.

As shown in FIGS. 1A, 1B, and 1C, patient 1000 may use the respiratory therapy system in a variety of positions. Accordingly, a position of patient interface 3000, for example, relative to air circuit 4170 or other aspects of the respiratory therapy system may vary throughout use.

### 5.3 Patient Interface

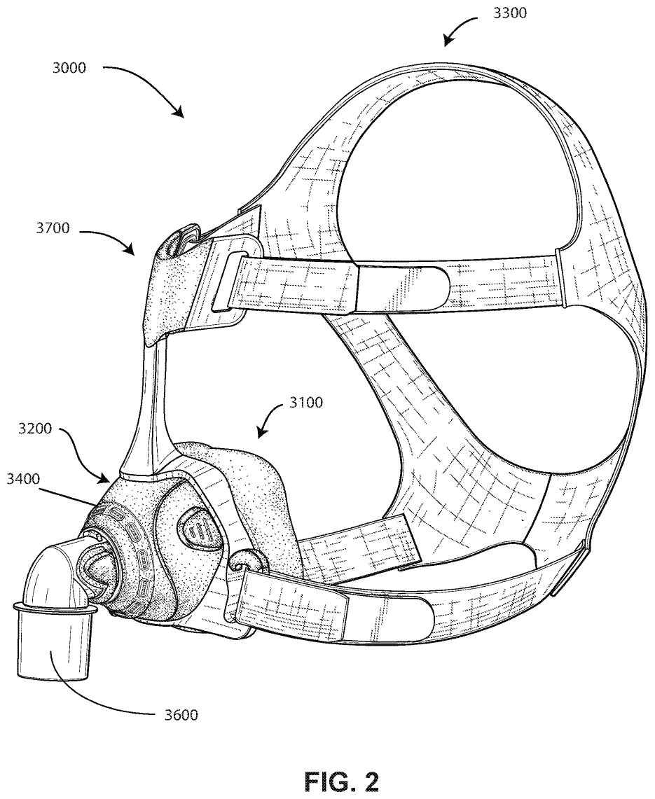

A non-invasive patient interface 3000, such as that shown in FIG. 2, in accordance with one aspect of the present technology comprises the following functional aspects: a seal-forming structure 3100, a plenum chamber 3200, a positioning and stabilising structure 3300, a vent 3400, one form of connection port 3600 for connection to air circuit 4170, and a forehead support-. In some forms a functional aspect may be provided by one or more physical components. In some forms, one physical component may provide one or more functional aspects. In use the seal-forming structure 3100 is arranged to surround an entrance to the airways of the patient so as to maintain positive pressure at the entrance(s) to the airways of the patient 1000. The sealed patient interface 3000 is therefore suitable for delivery of positive pressure therapy.

### 5.3.1 Plenum Chamber

The plenum chamber 3200 has a perimeter that is shaped to be complementary to the surface contour of the face of an average person in the region where a seal will form in use. In use, a marginal edge of the plenum chamber 3200 is positioned in close proximity to an adjacent surface of the face. Actual contact with the face is provided by the seal-forming structure 3100. The seal-forming structure 3100 may extend in use about the entire perimeter of the plenum chamber 3200. In some forms, the plenum chamber 3200 and the seal-forming structure 3100 are formed from a single homogeneous piece of material.

In certain forms of the present technology, the plenum chamber 3200 does not cover the eyes of the patient in use. In other words, the eyes are outside the pressurised volume defined by the plenum chamber. Such forms tend to be less obtrusive and/or more comfortable for the wearer, which can improve compliance with therapy.

In certain forms of the present technology, the plenum chamber 3200 is constructed from a transparent material, e.g. a transparent polycarbonate. The use of a transparent material can reduce the obtrusiveness of the patient interface, and help improve compliance with therapy. The use of a transparent material can aid a clinician to observe how the patient interface is located and functioning.

In certain forms of the present technology, the plenum chamber 3200 is constructed from a translucent material. The use of a translucent material can reduce the obtrusiveness of the patient interface, and help improve compliance with therapy.

In some forms, the plenum chamber 3200 is constructed from a rigid material such as polycarbonate. The rigid material may provide support to the seal-forming structure.

In some forms, the plenum chamber 3200 is constructed from a flexible material (e.g., constructed from a soft, flexible, resilient material like silicone, textile, foam, etc.). For example, in examples then may be formed from a material which has a Young's modulus of 0.4 Gpa or lower, for example foam. In some forms of the technology the plenum chamber 3200 may be made from a material having Young's modulus of 0.1 Gpa or lower, for example rubber. In other forms of the technology the plenum chamber 3200 may be made from a material having a Young's modulus of 0.4 Mpa or less, for example between 0.4 Mpa and 0.3 Mpa. An example of such a material is silicone.

### 5.3.1.1 Multiple Openings

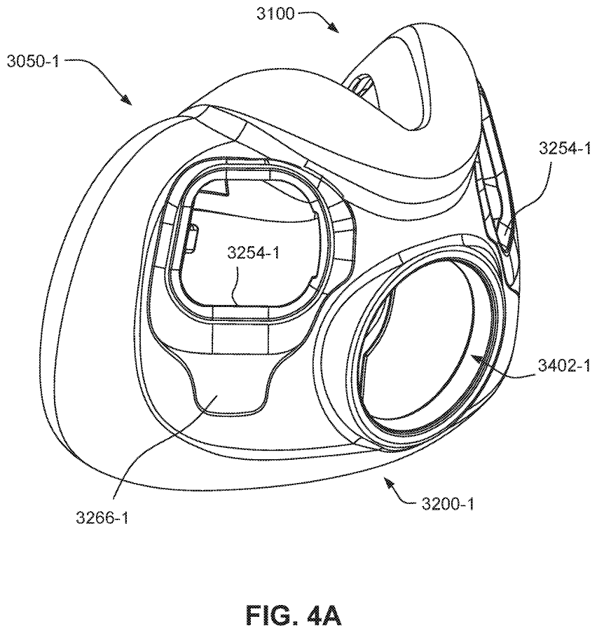

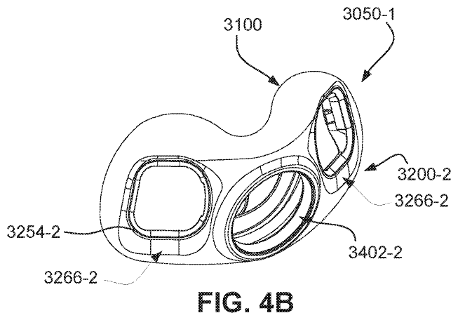

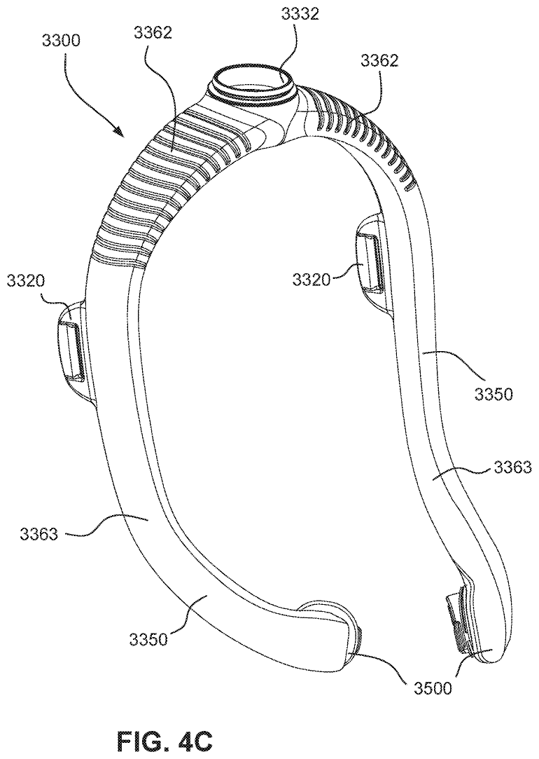

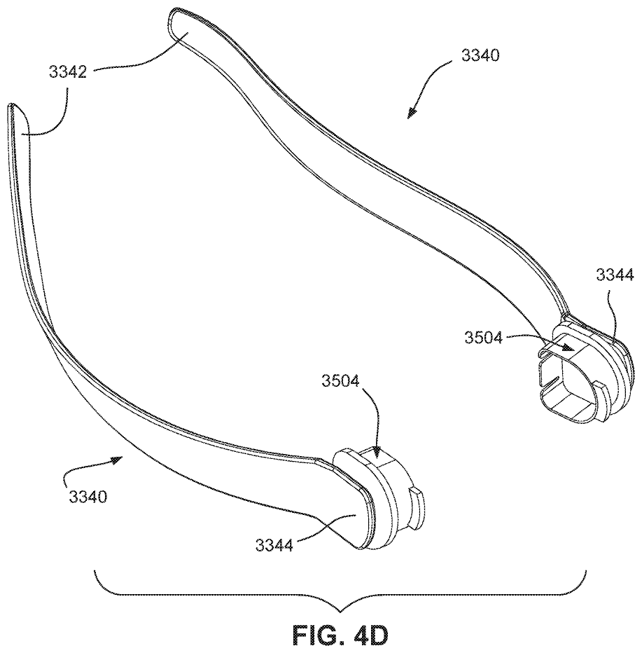

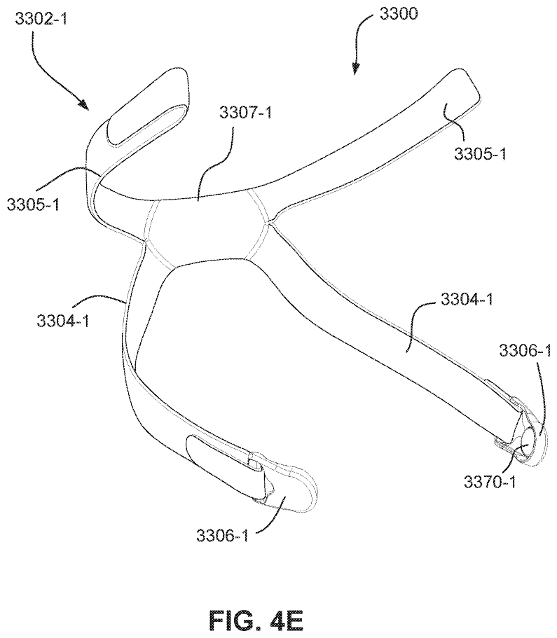

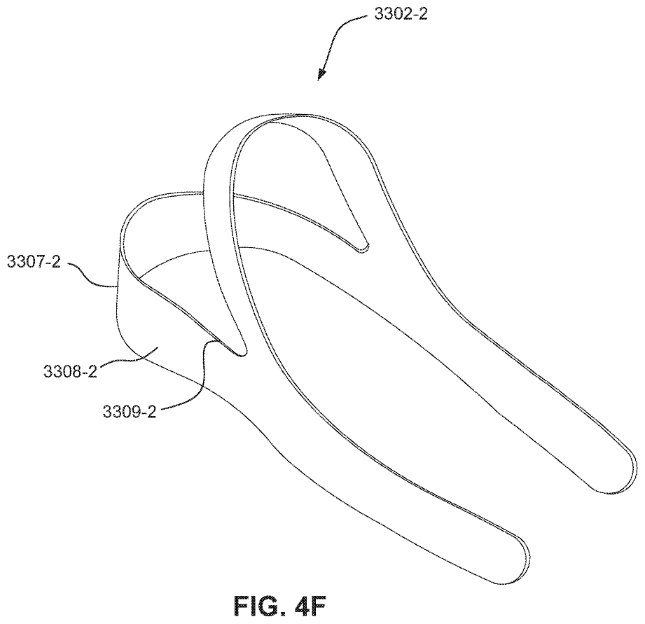

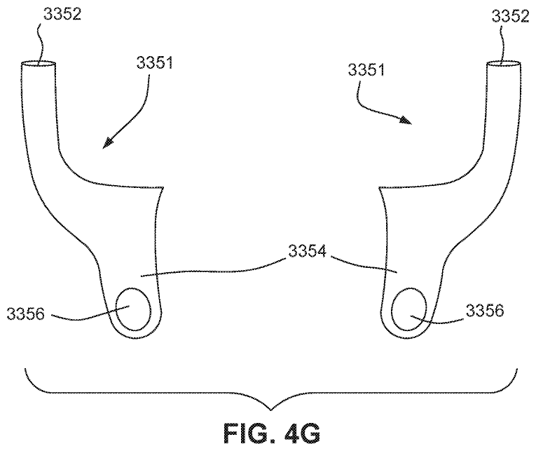

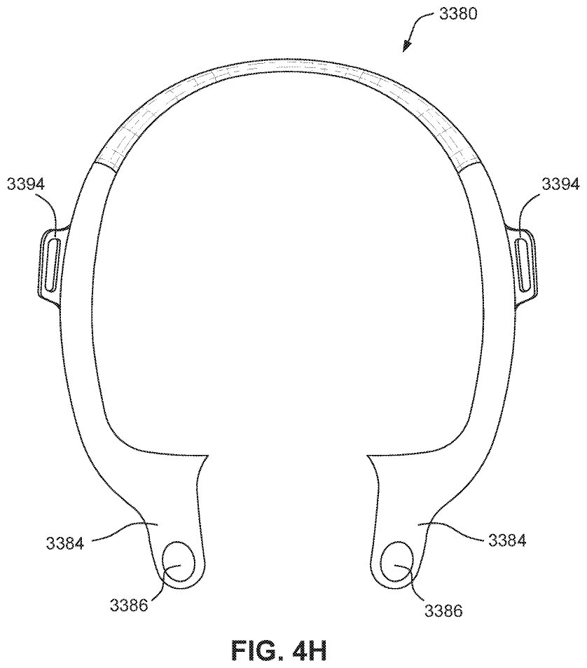

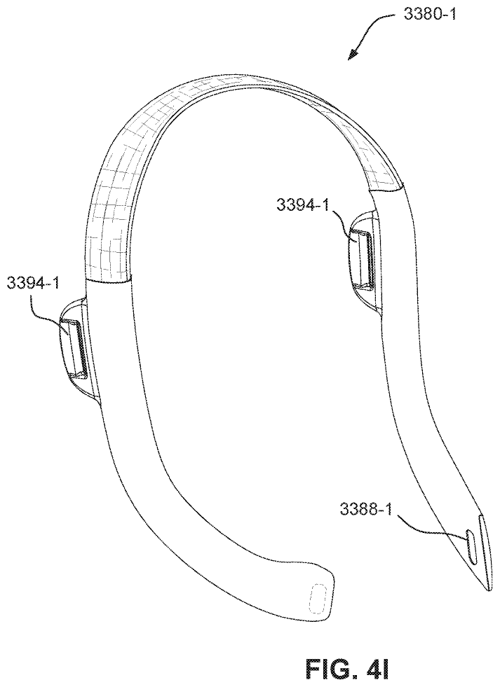

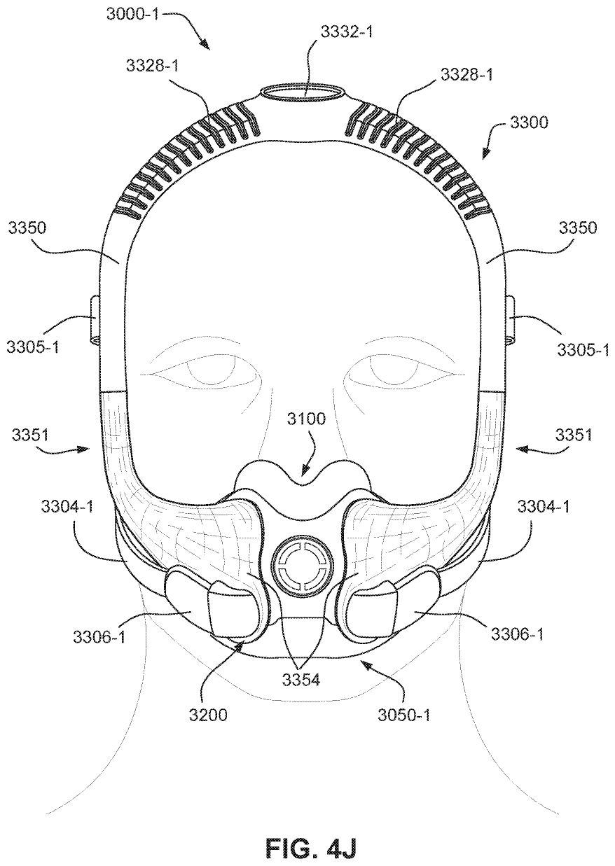

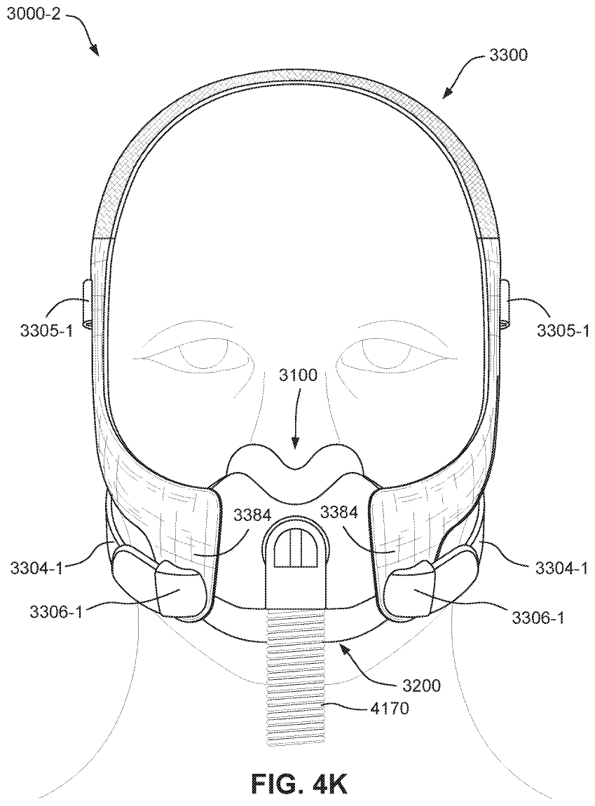

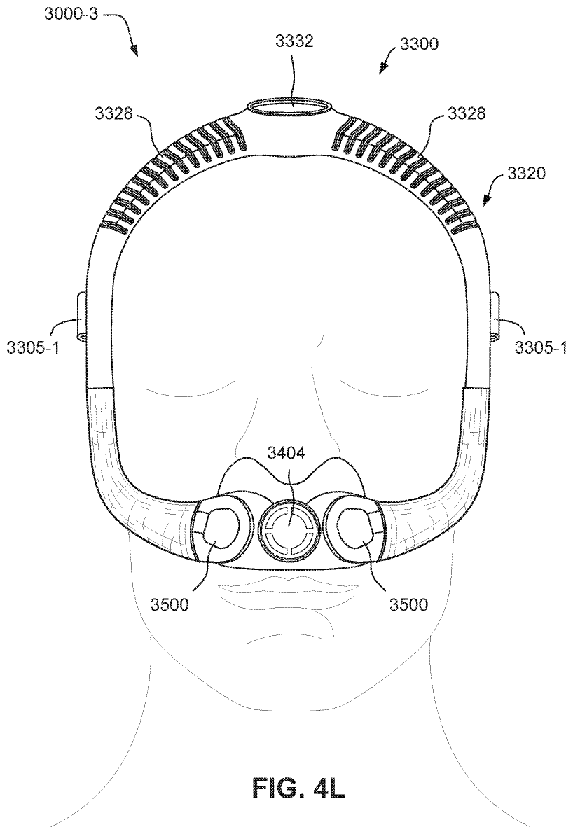

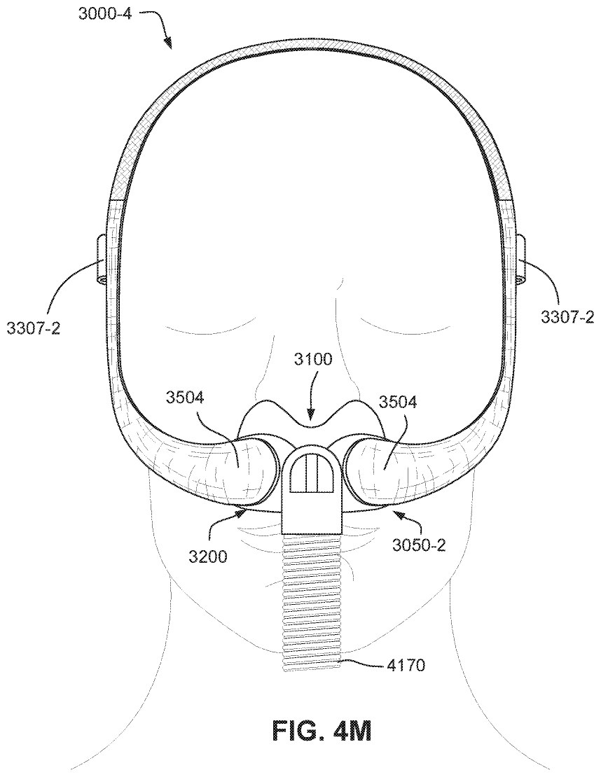

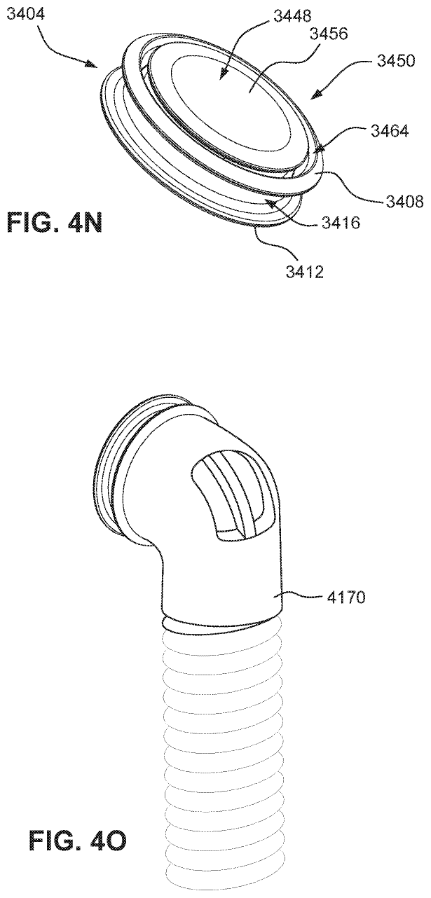

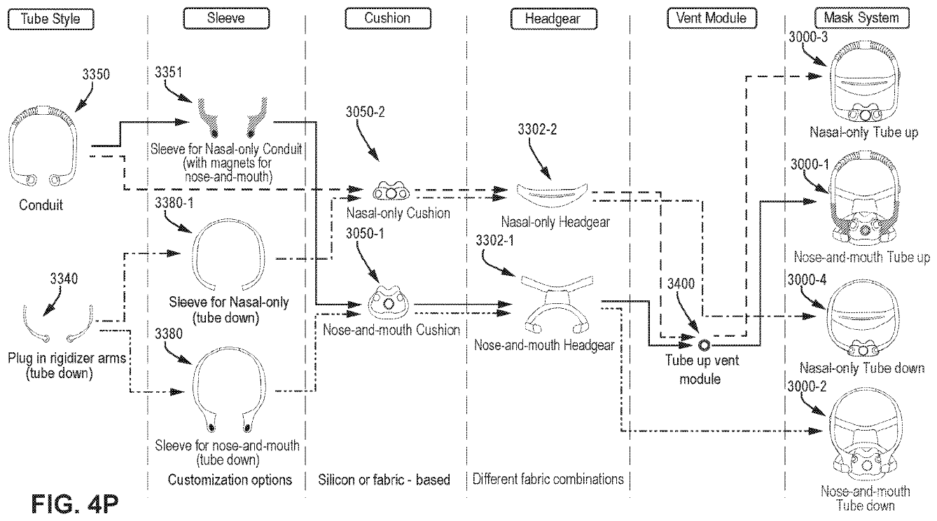

As shown in FIGS. 4A and 4B, different plenum chambers 3200-1, 3200-2 may be formed as part of a multi-opening cushion 3050-1, 3050-2. In the illustrated examples, the cushions 3050-1, 3050-2 each include three openings, although an alternate cushion may be formed with greater or fewer openings.

In some forms, the different openings may serve different functions. For example, some openings may be exclusively inlet openings, while other openings may be exclusively outlet openings.

In other forms, at least one opening may serve two different functions. For example, one opening may operate as both an inlet and an outlet during the same breathing cycle.

The plurality of openings may allow for a variety of configurations of air delivery to the plenum chamber 3200-1, 3200-2. For example, depending on patient need and/or patient comfort, the patient may use a given cushion 3050-1, 3050-2 in a “tube-up” configuration (e.g., using conduit headgear-described below) or a “tube-down” configuration (e.g., using a single conduit in front of the patient's face).

### 5.3.1.1.1 Nose and Mouth Mask

As shown in FIG. 4A, the plenum chamber 3200-1 includes a pair of plenum chamber inlet ports 3254-1, which may be used to convey gas into and/or out of the plenum chamber 3200-1. The plenum chamber inlet ports 3254-1 may be disposed on opposite sides (e.g., left and right sides) of the plenum chamber 3200-1.

In some forms, the plenum chamber 3200-1 may also include at least one vent opening 3402-1 (see e.g., FIG. 4A). The vent opening 3402-1 may be disposed in a center of the plenum chamber 3200-1. For example, the vent opening 3402-1 may be disposed between the plenum chamber inlet ports 3254-1.

In some forms, the plenum chamber 3200-1 may include a pair of grooves 3266-1. Each groove 3266-1 may be disposed proximate to one of the plenum chamber inlet ports 3254-1. Each groove 3266-1 may form a partially recessed surface.

### 5.3.1.1.2 Nose-Only Mask

The plenum chamber 3200-2 of a nasal only cushion 3050-2 may be similar to the plenum chamber 3200-1 of the mouth and nose cushion 3050-1. Only some similarities and differences between the plenum chambers 3200-1, 3200-2 may be described below.

As shown in FIG. 4B, the plenum chamber 3200-2 includes a pair of plenum chamber inlet ports 3254-2, which may be used to convey gas into and/or out of the plenum chamber 3200-2. The plenum chamber inlet ports 3254-2 may be disposed on opposite sides (e.g., left and right sides) of the plenum chamber 3200-2.

In some forms, the plenum chamber 3200-2 may also include at least one vent opening 3402-2 (see e.g., FIG. 4B). The vent opening 3402-2 may be disposed in a center of the plenum chamber 3200-2. For example, the vent opening 3402-2 may be disposed between the plenum chamber inlet ports 3254-2.

In some forms, the plenum chamber 3200-2 may include a pair of grooves 3266-2. Each groove 3266-2 may be disposed proximate to one of the plenum chamber inlet ports 3254-2. Each groove 3266-2 may form a partially recessed surface.

### 5.3.2 Positioning and Stabilising Structure

In one form the positioning and stabilising structure 3300 provides a retention force as a safety margin to overcome the potential effect of disrupting forces on the patient interface 3000, such as from tube drag, or accidental interference with the patient interface.

In one form of the present technology, a positioning and stabilising structure 3300 is provided that is configured in a manner consistent with being worn by a patient while sleeping. In one example the positioning and stabilising structure 3300 has a low profile, or cross-sectional thickness, to reduce the perceived or actual bulk of the apparatus. In one example, the positioning and stabilising structure 3300 comprises at least one strap having a rectangular cross-section. In one example the positioning and stabilising structure 3300 comprises at least one flat strap.

In one form of the present technology, a positioning and stabilising structure 3300 is provided that is configured so as not to be too large and bulky to prevent the patient from lying in a supine sleeping position with a back region of the patient's head on a pillow.

In one form of the present technology, a positioning and stabilising structure 3300 is provided that is configured so as not to be too large and bulky to prevent the patient from lying in a side sleeping position with a side region of the patient's head on a pillow.

In one form of the present technology, a positioning and stabilising structure 3300 is provided with a decoupling portion located between an anterior portion of the positioning and stabilising structure 3300, and a posterior portion of the positioning and stabilising structure 3300. The decoupling portion does not resist compression and may be, e.g. a flexible or floppy strap. The decoupling portion is constructed and arranged so that when the patient lies with their head on a pillow, the presence of the decoupling portion prevents a force on the posterior portion from being transmitted along the positioning and stabilising structure 3300 and disrupting the seal.

In one form of the present technology, a positioning and stabilising structure 3300 comprises a strap constructed from a laminate of a fabric patient-contacting layer, a foam inner layer and a fabric outer layer. In one form, the foam is porous to allow moisture, (e.g., sweat), to pass through the strap. In one form, the fabric outer layer comprises loop material to engage with a hook material portion.

In certain forms of the present technology, a positioning and stabilising structure 3300 comprises a strap that is extensible, e.g. resiliently extensible. For example the strap may be configured in use to be in tension, and to direct a force to draw a seal-forming structure into sealing contact with a portion of a patient's face. In an example the strap may be configured as a tie.

In one form of the present technology, the positioning and stabilising structure comprises a first tie, the first tie being constructed and arranged so that in use at least a portion of an inferior edge thereof passes superior to an otobasion superior of the patient's head and overlays a portion of a parietal bone without overlaying the occipital bone.

In one form of the present technology suitable for a nasal-only mask or for a full-face mask, the positioning and stabilising structure includes a second tie, the second tie being constructed and arranged so that in use at least a portion of a superior edge thereof passes inferior to an otobasion inferior of the patient's head and overlays or lies inferior to the occipital bone of the patient's head.

In one form of the present technology suitable for a nasal-only mask or for a full-face mask, the positioning and stabilising structure includes a third tie that is constructed and arranged to interconnect the first tie and the second tie to reduce a tendency of the first tie and the second tie to move apart from one another.

In certain forms of the present technology, a positioning and stabilising structure 3300 comprises a strap that is bendable and e.g. non-rigid. An advantage of this aspect is that the strap is more comfortable for a patient to lie upon while the patient is sleeping.

In certain forms of the present technology, a positioning and stabilising structure 3300 comprises a strap constructed to be breathable to allow moisture vapour to be transmitted through the strap,

In certain forms of the present technology, a system is provided comprising more than one positioning and stabilising structure 3300, each being configured to provide a retaining force to correspond to a different size and/or shape range. For example the system may comprise one form of positioning and stabilising structure 3300 suitable for a large sized head, but not a small sized head, and another. Suitable for a small sized head, but not a large sized head.

### 5.3.2.1 Conduit Headgear

### 5.3.2.1.1 Conduit Headgear Tubes

In some forms of the present technology, the positioning and stabilising structure 3300 comprises one or more headgear tubes 3350 that deliver pressurised air received from a conduit forming part of the air circuit 4170 from the RPT device to the patient's airways, for example through the plenum chamber 3200 and seal-forming structure 3100. In the form of the present technology illustrated in FIG. 4J, the positioning and stabilising structure 3300 comprises two tubes 3350 that deliver air to the plenum chamber 3200 from the air circuit 4170. The tubes 3350 are configured to position and stabilise the seal-forming structure 3100 of the patient interface 3000 at the appropriate part of the patient's face (for example, the nose and/or mouth) in use. This allows the conduit of air circuit 4170 providing the flow of pressurised air to connect to a connection port 3600 of the patient interface in a position other than in front of the patient's face, for example on top of the patient's head.

In the form of the present technology illustrated in FIG. 4J, the positioning and stabilising structure 3300 comprises two tubes 3350, each tube 3350 being positioned in use on a different side of the patient's head and extending across the respective cheek region, above the respective ear (superior to the otobasion superior on the patient's head) to the elbow 3610 on top of the head of the patient 1000. This form of technology may be advantageous because, if a patient sleeps with their head on its side and one of the tubes 3350 is compressed to block or partially block the flow of gas along the tube 3350, the other tube 3350 remains open to supply pressurised gas to the patient. In other examples of the technology, the patient interface 3000 may comprise a different number of tubes, for example one tube, or two or more tubes.

In one example in which the patient interface has one tube 3350, the single tube 3350 is positioned on one side of the patient's head in use (e.g. across one cheek region) and a strap forms part of the positioning and stabilising structure 3300 and is positioned on the other side of the patient's head in use (e.g. across the other region) to assist in securing the patient interface 3000 on the patient's head. For example, the tube 3350 and the strap may each be under tension in use in order to assist in maintaining the seal-forming structure 3100 in a sealing position.

In one form, the tube 3350 may be at least partially extensible so that the tube 3350 and the strap may adjust substantially equal lengths when worn by a patient. This may allow for substantially symmetrical adjustments between the tube 3350 and the strap so that the seal-forming structure remains substantially in the middle.

In the form of the technology shown in FIG. 4J, the two tubes 3350 are fluidly connected at superior ends to each other and to the connection port 3600. In some examples, the two tubes 3350 are integrally formed while in other examples the tubes 3350 are formed separately but are connected in use and may be disconnected, for example for cleaning or storage. Where separate tubes are used, they may be indirectly connected together, for example each may be connected to a T-shaped connector. The T-shaped connector may have two arms/branches each fluidly connectable to a respective one of the tubes 3350. Additionally, the T-shaped connector may have a third arm or opening providing the connection port 3600 for fluid connection to the air circuit 4170 in use. The opening may be an inlet 3332 (see e.g., 4C) for receiving the flow of pressurized air.

In some forms, the third arm of the T-shaped connector may be substantially perpendicular to each of the first two arms.

In some forms, the third arm of the T-shaped connector may be obliquely formed with respect to each of the first two arms.

In some forms, a Y-shaped connector may be used instead of the T-shaped connector. The first two arms may be oblique with respect to one another, and the third arm may be oblique with respect to the first two arms. The angled formation of the first two arms may be similar to the shape of the patient's head in order to conform to the shape.

In some forms, at least one of the arms of the T-shaped connector (or Y-shaped connector) may be flexible. This may allow the connector to bend based on the shape of the patient's head and/or a force in the positioning and stabilising structure 3300.

In some forms, at least one of the arms of the T-shaped connector (or Y-shaped connector) may be at least partially rigidised. This may assist in maintaining the shape of the connector so that bending of the connector does not close the airflow path.

The tubes 3350 may be formed from a flexible material, such as an elastomer, e.g. silicone or TPE, and/or from one or more textile and/or foam materials. The tubes 3350 may have a preformed shape and may be able to be bent or moved into another shape upon application of a force but may return to the original preformed shape in the absence of said force. The tubes 3350 may be generally arcuate or curved in a shape approximating the contours of a patient's head between the top of the head and the nasal or oral region.

In some examples, the one or more tubes 3350 are crush resistant to resist being blocked if crushed during use, for example if squashed between a patient's head and pillow, especially if there is only one tube 3350. The tubes 3350 may be formed with a sufficient structural stiffness to resist crushing or may be as described in U.S. Pat. No. 6,044,844, the contents of which are incorporated herein by reference.

Each tube 3350 may be configured to receive a flow of air from the connection port 3600 on top of the patient's head and to deliver the flow of air to the seal-forming structure 3100 at the entrance of the patient's airways. In the example shown in FIG. 4J, each tube 3350 lies in use on a path extending from the plenum chamber 3200 across the patient's cheek region and superior to the patient's ear to the elbow 3610. For example, a portion of each tube 3350 proximate the plenum chamber 3200 may overlie a maxilla region of the patient's head in use. Another portion of each tube 3350 may overlie a region of the patient's head superior to an otobasion superior of the patient's head. Each of the tubes 3350 may also lie over the patient's sphenoid bone and/or temporal bone and either or both of the patient's frontal bone and parietal bone. The elbow 3610 may be located in use over the patient's parietal bone, over the frontal bone and/or over the junction therebetween (e.g. the coronal suture).

In certain forms of the present technology the patient interface 3000 is configured such that the connection port 3600 can be positioned in a range of positions across the top of the patient's head so that the patient interface 3000 can be positioned as appropriate for the comfort or fit of an individual patient. In some examples, the headgear tubes 3350 are configured to allow movement of an upper portion of the patient interface 3000 (e.g. a connection port 3600) with respect to a lower portion of the patient interface 3000 (e.g. a plenum chamber 3200). That is, the connection port 3600 may be at least partially decoupled from the plenum chamber 3200. In this way, the seal-forming structure 3100 may form an effective seal with the patient's face irrespective of the position of the connection port 3600 (at least within a predetermined range of positions) on the patient's head.

As described above, in some examples of the present technology the patient interface 3000 comprises a seal-forming structure 3100 in the form of a cradle cushion which lies generally under the nose and seals to an inferior periphery of the nose (e.g. an under-the-nose cushion). The positioning and stabilising structure 3300, including the tubes 3350 may be structured and arranged to pull the seal-forming structure 3100 into the patient's face under the nose with a sealing force in a posterior and superior direction (e.g. a posterosuperior direction). A sealing force with a posterosuperior direction may cause the seal-forming structure 3100 to form a good seal to both the inferior periphery of the patient's nose and anterior-facing surfaces of the patient's face, for example on either side of the patient's nose and the patient's lip superior.

### 5.3.2.1.2 Extendable and Non-Extendable Tube Portions

In some examples of the present technology, one or both of the tubes 3350 are not extendable in length. However, in some forms, the tubes 3350 may comprise one or more extendable tube sections, for example formed by an extendable concertina structure. In some forms, the patient interface 3000 may comprise a positioning and stabilising structure 3300 including at least one gas delivery tube comprising a tube wall having an extendable concertina structure. The patient interface 3000-1 shown in FIG. 4J comprises tubes 3350, the superior portions of which comprise extendable tube sections each in the form of an extendable concertina structure 3328-1. In the illustrated example, the superior portion of each of the tubes 3350 includes the extendable concertina structure 3328-1. In an alternative example, the superior portion of the tubes 3350 may not include the extendable concertina structure 3328-1.

In some forms, the extendable concertina structure 3328 (e.g., see FIGS. 4J and 4L) may be formed as a series of ridges and grooves on the surface of the tubes 3350. The concertina structure 3328 may be biased toward a retracted position, and may move to an expanded position when the patient dons the positioning and stabilising structure 3300. Because portions of the tubes 3350 may be substantially inextensible (e.g., non-extendable tube sections 3363), the concertina structures 3328 permit the positioning and stabilising structure 3300 to stretch in order to fit different sized heads. This may allow a single sized tube 3350 to be used with multiple sized heads. For example, the positioning and stabilising structure 3300 may be “one-size-fits-all” as a result of the concertina structure 3328. Alternatively, the tubes 3350 may be manufactured in multiple sizes (e.g., small, medium, large). The patient may select a length that most closely conforms to their head, and the concertina structures 3328 may make small adjustments in order to tailor the fit to the individual patient.

In some forms, the inlet 3332 may be disposed in the middle of the conduit 6320. For example, the tubes 3350 may be symmetric about the inlet 3332 through at least one axis.

The cross-sectional shape of the non-extendable tube sections 3363 of the tubes 3350 may be circular, elliptical, oval, D-shaped or a rounded rectangle, for example as described in U.S. Pat. No. 6,044,844. A cross-sectional shape that presents a flattened surface of tube on the side that faces and contacts the patient's face or other part of the head may be more comfortable to wear than, for example a tube with a circular cross-section.

In some examples of the present technology, the non-extendable tube sections 3363 connects to the plenum chamber 3200 from a low angle. The headgear tubes 3350 may extend inferiorly down the sides of the patient's head and then curve anteriorly and medially to connect to the plenum chamber 3200 in front of the patient's face. The tubes 3350, before connecting to the plenum chamber 3200, may extend to a location at the same vertical position as (or, in some examples, inferior to) the connection with the plenum chamber 3200. That is, the tubes 3350 may project in an at least partially superior direction before connecting with the plenum chamber 3200. A portion of the tubes 3350 may be located inferior to the plenum chamber 3200 and/or the seal-forming structure 3100. The tubes 3350 may contact the patient's face below the patient's cheekbones, which may be more comfortable than contact on the patient's cheekbones and may avoid excessively obscuring the patient's peripheral vision.

### 5.3.2.1.3 Conduit Headgear Connection Port

In certain forms of the present technology, the patient interface 3000 may comprise a connection port 3600 located proximal to a superior, lateral or posterior portion of a patient's head. For example, in the form of the present technology illustrated in FIG. 4J, the connection port 3600 is located on top of the patient's head (e.g. at a superior location with respect to the patient's head). In this example the patient interface 3000 comprises an elbow 3610 forming the connection port 3600. The elbow 3610 may be configured to fluidly connect with a conduit of an air circuit 4170. The elbow 3610 may be configured to swivel with respect to the positioning and stabilising structure 3300 to at least partially decouple the conduit from the positioning and stabilising structure 3300. In some examples the elbow 3610 may be configured to swivel by rotation about a substantially vertical axis and, in some particular examples, by rotation about two or more axes. In some examples the elbow may comprise or be connected to the tubes 3350 by a ball-and-socket joint. The connection port 3600 may be located in the sagittal plane of the patient's head in use.

Patient interfaces having a connection port that is not positioned anterior to the patient's face may be advantageous as some patients may find a conduit that connects to a patient interface anterior to their face to be unsightly and/or obtrusive. For example, a conduit connecting to a patient interface anterior to the patient's face may be prone to interference with bedclothes or bed linen, particularly if the conduit extends inferiorly from the patient interface in use. Forms of the present technology comprising a patient interface having a connection port positioned superiorly to the patient's head in use may make it easier or more comfortable for a patient to lie or sleep in one or more of the following positions: a side-sleeping position, a supine position (e.g. on their back, facing generally upwards) or in a prone position (e.g. on their front, facing generally downwards). Moreover, connecting a conduit to an anterior portion of a patient interface may exacerbate a problem known as tube drag in which the conduit exerts an undesired force upon the patient interface during movement of the patient's head or the conduit, thereby causing dislodgement away from the face. Tube drag may be less of a problem when force is received at a superior location of the patient's head than anterior to the patient's face proximate to the seal-forming structure (where tube drag forces may be more likely to disrupt the seal).

### 5.3.2.1.4 Headgear Tube Fluid Connections

The two tubes 3350 are fluidly connected at their inferior ends to the plenum chamber 3200. In certain forms of the technology, the connection between the tubes 3350 and the plenum chamber 3200 is achieved by connection of two rigid connectors. The tubes 3350 and plenum chamber 3200 may be configured to enable the patient to easily connect the two components together in a reliable manner. The tubes 3350 and plenum chamber 3200 may be configured to provide tactile and/or audible feedback in the form of a ‘re-assuring click’ or a similar sound, so that the patient may easily know that each tube 3350 has been correctly connected to the plenum chamber 3200. In one form, the tubes 3350 are formed from a silicone or textile material and the inferior end of each of the silicone tubes 3350 is overmolded to a rigid connector made, for example, from polypropylene, polycarbonate, nylon or the like. The rigid connector on each tube 3350 may comprise a female mating feature configured to connect with a male mating feature on the plenum chamber 3200. Alternatively, the rigid connector on each tube 3350 may comprise a male mating feature configured to connect to a female mating feature on the plenum chamber 3200. In other examples the tubes 3350 may each comprise a male or female connector formed from a flexible material, such as silicone or TPE, for example the same material from which the tubes 3350 are formed.

In other examples a compression seal is used to connect each tube 3350 to the plenum chamber 3200. For example, a resiliently flexible (e.g. silicone) tube 3350 without a rigid connector may be configured to be squeezed to reduce its diameter so that it can be compressed into a port in the plenum chamber 3200 and the inherent resilience of the silicone pushes the tube 3350 outwards to seal the tube 3350 in the port in an air-tight manner. Alternatively, in a hard-to-hard type engagement between the tube 3350 and the plenum chamber 3200, each tube 3350 and/or plenum chamber 3200 may comprise a pressure activated seal, for example a peripheral sealing flange. When pressurised gas is supplied through the tubes 3350 the sealing flange may be urged against the join between the tubes and a circumferential surface around a port or connector of the plenum chamber 3200 to form or enhance a seal between the tube 3350 and plenum chamber 3200.

### 5.3.2.2 Headgear Straps

In some forms, the positioning and stabilising structure 3300 may include headgear 3302 with at least one strap which may be worn by the patient in order to assist in properly orienting the seal-forming structure 3100 against the patient's face (e.g., in order to limit or prevent leaks).

As described above, some forms of the headgear 3302 may be constructed from a textile material, which may be comfortable against the patient's skin. The textile may be flexible in order to conform to a variety of facial contours. Although the textile may include rigidisers along a selected length, which may limit bending, flexing, and/or stretching of the headgear 3302.

In certain forms, the headgear 3302 may be at least partially extensible. For example, the headgear 3302 may include elastic, or a similar extensible material. For example, the entire headgear 3302 may be extensible or selected portions may be extensible (or more extensible than surrounding portions). This may allow the headgear 3302 to stretch while under tension, which may assist in providing a sealing force for the seal-forming structure 3100.

Two forms of the headgear, four-point headgear 3302-1 and two-point headgear 3302-2, are discussed in more detail below as illustrative examples.

### 5.3.2.2.1 Four-Point Connection

As shown in FIG. 4E, some forms of the headgear 3302-1 may be a four-point connection headgear. This means that the headgear 3302-1 may connect to four separate places on the plenum chamber 3200, on a frame connected to the plenum chamber 3200, and/or on arms connected to the plenum chamber 3200. The headgear 3302-1 may include four different straps providing a tensile force to help maintain the seal-forming structure 3100 in a sealing position.

In some forms, the headgear 3302-1 may include inferior straps 3304-1, which may connect to an inferior portion of the cushion 3050-1. The inferior straps 3304-1 may extend along the patient's cheek toward a posterior region of the patient's head. For example, the inferior straps 3304-1 may overlay the masseter muscle on either side of the patient's face. The inferior straps 3304-1 may therefore contact the patient's head below the patient's ears. The inferior straps 3304-1 may meet at the posterior of the patient's head, and may overlay the occipital bone and/or the trapezius muscle.

The headgear 3302-1 may also include superior straps 3305-1, which may overlay the temporal bones, parietal bone, and/or occipital bone. The superior straps 3305-1 may also connect to the tubes 3350 (e.g., by interfacing with the tabs 3320).

A rear strap 3307-1 may extend between the superior straps 3305-1 and between the inferior straps 3304-1. The inferior and superior straps 3304-1, 3305-1 on a given side (e.g., left or right) may also be connected to the rear strap 3307-1 adjacent to one another. The height of the rear strap 3307-1 may therefore be approximately the combined height of the inferior and superior strap 3304-1, 3305-1. The rear strap 3307-1 may overlay the occipital bone and/or the parietal bone in use. This may allow the rear strap 3307-1 to assist in anchoring the headgear 3302-1 to the patient's head.

In the illustrated example, the headgear 3302-1 may be formed with a substantially X-shape. The inferior and superior straps 3304-1, 3305-1 may be connected to a rear strap 3307-1 using stitching, ultrasonic welding, or any similar process.

In some forms, the inferior straps 3304-1 are connected to a magnetic member 3306-1. For example, each inferior straps 3304-1 may be threaded through a magnetic member 3306-1, so that a length of each inferior strap 3304-1 may be adjusted. The magnetic members 3306-1 may removably connect to the magnets 3370-1 (described below), so that the inferior straps 3304-1 may be disconnected from the plenum chamber 3200, but the length of the inferior straps 3304-1 may not be affected.

In some forms, the superior straps 3305-1 may be connected directly to the tabs 3320 of the tubes 3350. The superior straps 3305-1 may be threaded through the tabs 3320 in order to adjust the length and control the tensile force of each superior strap 3305-1.

In some forms, the headgear 3302-1 may be used only with the nose and mouth cushion 3050-1 (e.g., because the nose-only cushion 3050-1 does not have four connection points). However, the headgear 3302-1 may be used interchangeably with the tubes 3350 and the rigidiser arms 3340.

### 5.3.2.2.2 Two-Point Connection

As shown in FIG. 4F, some forms of the headgear 3302-2 may be a two-point connection headgear. This means that the headgear 3302-2 may connect to two separate places.

In some forms, the headgear 3302-2 may be formed from a continuous piece of material. In other words, the headgear 3302-2 may not be formed from multiple straps connected (e.g., stitched) together. This may be comfortable for a patient as they will not be in contact with any seams or joints connecting different straps. In other forms, the headgear 3302-2 may be formed from multiple straps (e.g., two superior straps, a rear strap, etc.) that are connected together (e.g., with stitching, ultra-sonic welding, etc.).

In certain forms of the present technology, the positioning and stabilising structure 3300 comprises at least one headgear strap acting in addition to the tubes 3350 to position and stabilise the seal-forming structure 3100 at the entrance to the patient's airways. As shown in FIG. 4F, the patient interface 3000 comprises a rear strap 3307-2 forming part of the positioning and stabilising structure 3300. The rear strap 3307-2 may be known as a back strap or a rear headgear strap, for example. The rear strap 3307-2 may overlay the temporal bones, parietal bone, and/or occipital bone. In other examples of the present technology, one or more further straps may be provided. For example, patient interface 3000 according to examples of the present technology having a nose-and-mouth cushion may have a second, lower, strap configured to lie against the patient's head proximate the patient's neck and/or against posterior surfaces of the patient's neck.

As shown in FIG. 4F, some forms of the headgear 3302-2 may be at least partially bifurcated. For example, a rear strap 3307-2 of the headgear 3302-2 (e.g., configured to contact the posterior portion of the patient's head) may be wider than the surrounding portions of the headgear 3302-2. An intermediate section 3308-2 of the rear strap 3307-2 may include a slit 3309-2. A superior section of the rear strap 3307-2 may therefore be movable relative to the inferior section as a result of the slit 3309-2. This may allow the patient to have a larger strap coverage on the posterior region of their head, which may assist in better anchoring the headgear 3302-2 to the patient's head since there is no inferior strap (e.g., 3304-1).

In some forms, the headgear 3302-2 may be used only with the nasal cushion 3050-2 (e.g., because the nose and mouth cushion 3050-1 does not have four connection points). However, the headgear 3302-2 may be used interchangeably with the tubes 3350 and the rigidiser arms 3340.

### 5.3.2.3 Rigidiser Arm

As shown in FIG. 4D, a rigidiser arm 3340 may be an elongated, rigid member that assists in maintaining the cushion (e.g., the nose and mouth cushion 3050-1 or the nasal cushion 3050-2) in an operating position. The rigidiser arm 3340 may contact a side of the patient's head and provide a force to limit slipping of the seal-forming structure 3100 from the patient's nose and/or mouth.

In some forms, the rigidiser arm 3340 is constructed from a rigid material (e.g., plastic). The rigid material may not permit the rigidiser arm 3340 to stretch.

In some forms, the rigidiser arm 3340 may be flexible along at least one direction. For example, the rigidiser arm 3340 may be flexible about its width and may be inflexible along its length. In other words, the rigidiser arm 3340 may be bendable about an axis along the width of the rigidiser arm 3340, but may be unable to bend about an axis perpendicular to the rigidiser arm 3340. This may allow an individual patient to adjust the rigidiser arm 3340 in order to better fit their individual head.

In certain forms, the rigidiser arm 3340 may remain in the new position after being bent. This may allow a patient to adjust the shape of the rigidiser arm 3340 for their specific head and then the rigidiser arm 3340 will keep the desired shape while in use in order to promote patient comfort.

In some forms, a first end 3342 of the rigidiser arm 3340 may be a free end and a second end 3344 (e.g., opposite of the first end 3342) of the rigidiser arm 3340 may be fixed. The first end 3342 may be curved in order to minimize sharp edges that could cause patient discomfort. The first end 3342 may also overlay the patient's head proximate to the temporal bone, in use. The second end 3344 may be fixed to an arm connection structure 3504.

In some forms, the arm connection structure 3504 may be similar to the conduit connection structure 3500. For example, the arm connection structure 3504 and the conduit connection structure 3500 may have substantially the same shape. This may allow either the conduit connection structure 3500 or the arm connection structure 3504 to fit into the groove (e.g., 3266-1 or 3266-2) and connect to the plenum chamber inlet port 3254. The arm connection structure 3504 may connect to the nose and mouth cushion 3050-1 or the nose-only cushion 3050-2 in substantially the same way as the conduit connection structure 3500 (e.g., via a snap fit, press fit, friction fit, etc.).

In some forms, the arm connection structure 3504 may act as a plug for the plenum chamber inlet port 3254 (e.g., either 3254-1 and/or 3254-2). Unlike the tubes 3350, the rigidiser arm 3340 does not convey pressurized air to the plenum chamber 3200. The rigidiser arm 3340 may be used with a “tube down” configuration, where a hose is connected to the vent opening 3402 (e.g., either 3402-1 and/or 3402-2), and conveys air into the plenum chamber 3200 through the vent opening 3402. In this example, air does not need to travel into or out of the plenum chamber inlet ports 3254. Thus, the arm connection structure 3504 may form a seal with the plenum chamber inlet port 3254 in order to limit airflow into or out of the plenum chamber 3200.

### 5.3.3 Vent

In one form, the patient interface 3000 includes a vent 3400 constructed and arranged to allow for the washout of exhaled gases, e.g. carbon dioxide.

In certain forms the vent 3400 is configured to allow a continuous vent flow from an interior of the plenum chamber 3200 to ambient whilst the pressure within the plenum chamber is positive with respect to ambient. The vent 3400 is configured such that the vent flow rate has a magnitude sufficient to reduce rebreathing of exhaled CO2 by the patient while maintaining the therapeutic pressure in the plenum chamber in use.

One form of vent 3400 in accordance with the present technology comprises a plurality of holes, for example, about 20 to about 80 holes, or about 40 to about 60 holes, or about 45 to about 55 holes.

The vent 3400 may be located in the plenum chamber 3200. Alternatively, the vent 3400 is located in a decoupling structure, e.g., a swivel.

As shown in FIG. 4N, a vent 3450 may be used with the patient interface 3000. The vent 3450 may have a substantially similar shape to the vent opening 3402-1 (e.g., a substantially circular shape).

The vent 3450 may be used with either the mouth and nose plenum chamber 3200-1 (e.g., illustrated in FIG. 4A) or the nose-only plenum chamber 3200-2 (e.g., illustrated in FIG. 4B).

With continued reference to FIG. 4A, the vent 3450 may include a vent housing 3404, which may be configured to engage with the vent opening 3402. The vent housing 3404 may be constructed from a rigid material or a semi-rigid material. For example, the vent housing 3404 may be constructed from plastic, metal, or any similar material. The vent housing 3404 may add rigidity to the patient interface 3000 (e.g., to limit unwanted bending that may affect the position of the seal-forming structure 3100 on the patient's face).

The vent housing 3404 may include an anterior surface 3408, a posterior surface 3412, and a groove 3416. The anterior surface 3408 faces away from the patient's face in use, and may be positioned outside the pressurized volume of the plenum chamber 3200. The posterior surface 3412 is disposed opposite to the anterior surface 3408. In use, the posterior surface 3412 may face the patient and may be disposed within the pressurized volume of the plenum chamber 3200. The groove 3416 may be formed between the anterior and posterior surfaces 3408, 3412. A portion of the plenum chamber 3200 may be received within the groove 3416 in order to retain the vent 3400 in position.

In some forms, a diffuser 3448 may be used with the vent housing 3404. The diffuser 3448 may assist with limiting the decibel output from any of the patient interface 3000 (or any other patient interface). Specifically, the diffuser 3448 may assist in limiting the decibel level associated with air output from the patient interface 3000 (e.g., exhaled air), although the diffuser 3448 may limit the decibel level of at any point in the patient interface.

In certain forms, the diffuser 3448 may diffuse, and therefore slow, the exhaust gas exiting the plenum chamber 3200 and passing through the vent housing 3404. The diffuser 3448 may assist in avoiding jetting and associated discomfort to the patient and/or bed partner (e.g., noise caused by jetting against a pillow, sheets, bedclothes, etc.).

In some forms, the diffuser may include an anterior surface 3456 that faces away from the patient in use. An outer diameter of the anterior surface 3456 may be less than an inner diameter of the vent housing 3404 proximate to the anterior surface 3408.

This may form a gap 3464 through which air may travel.

### 5.3.4 Decoupling Structure(s)

In one form the patient interface 3000 includes at least one decoupling structure, for example, a swivel or a ball and socket.

### 5.3.5 Modularity

As described above, the cushion, headgear, and sleeves may come in different styles, which may correspond to different uses (e.g., mouth breathing, nasal breathing, etc.). A patient or clinician may select certain combinations of cushions, headgear, and sleeves in order to optimize the effectiveness of the therapy and/or the individual patient's comfort. An example of this sort of modular design is described in PCT/SG2022/050777 filed 28 Oct. 2022, incorporated herein by reference in its entirety.

In some forms, the different styles of cushions, headgear, and sleeves may be used interchangeably with one another in order to form different combinations of patient interfaces. This may be beneficial from a manufacturing perspective because wider variety of patient interfaces may be created using fewer parts. Additionally or alternatively, the various combinations may allow a patient to change styles of patient interface without changing every component.

Air may be delivered to the patient in one of two main ways. In one example, the patient may receive the flow of pressurized air through headgear tubes 3350 (see e.g., FIG. 4C, 4J). This may be referred to as a “tube up” configuration and may position a connection port at the top of the patient's head. In other example, the patient may receive the flow of pressurized air through a conduit connected to the plenum chamber 3200. This may be referred to a “tube down” configuration where the airflow conduit is positioned in front of the patient's face. Different patients may be more comfortable with one style of air delivery over the other (e.g., because of the patient's sleep style). Therefore, it may be beneficial to allow a single style of patient interface to be used in either the “tube up” or “tube down” configuration.

The patient interface may be part of a modular assembly with a variety of interchangeable components that may be swapped out by a patient and/or clinician for one or more components for a different style. The following description describes the various combinations that may be created by assembling the different components together.

### 5.3.5.1 Sleeve

In some forms, to allow for modularity, a sleeve may be used with the tubes 3350 and/or the rigidiser arms 3340. The sleeve may at least partially surround the tubes 3350 and/or the rigidiser arms 3340. As shown in FIGS. 4G to 4I, different shapes of sleeves may be used, which may correspond to different types of positioning and stabilising structures 3300. In some forms, the configuration of the sleeve may be customized to fit a particular user's face. For instance, the sleeves may be configured in a relatively more posterior region of the patient's head.

In some forms, the sleeve may be constructed from a comfortable material. For example, the sleeve may be constructed from a textile material, a foam material, or a combination of the two. The comfortable material may contact the patient in use, and may feel soft against the patient's skin in order to improve patient compliance.

The material may also be flexible in order to assist in donning or doffing the sleeve from the tube 3350 or the rigidiser arms 3340. For example, the material may allow the sleeve to bend in order to conform to the shape of the tubes 3350 or conduit headgear or the rigidiser arms 3340, which may change depending on the shape of an individual patient's head.

In some forms, the sleeve may also be at least partially elastic (e.g., the material may allow the sleeve to stretch). The elastic material may help the sleeve stretch in order to fit around the tubes 3350 or the rigidiser arms 3340. The elastic material may then return to an initial position that is snug against the tubes 3350 or the rigidiser arms 3340 in order to limit the sleeve from sliding while in use.

As described in more detail below, some forms of the sleeves may be specific to a rigidising element (e.g., tubes 3350 and/or rigidiser arms 3340). However, the sleeves may assist the rigidising elements in connecting interchangeably with the version or styles of cushions (e.g., the mouth and nose cushion 3050-1, the nose-only cushion 3050-2, etc.).

### 5.3.5.2 Conduit Sleeve

As shown in FIG. 4G, one example of a sleeve is a conduit sleeve 3351, which may be usable with the tubes 3350 described above.

As shown in FIG. 4G, the conduit sleeve 3351 may include a curved shape that may be similar to the shape of the tubes 3350 shown in FIG. 4C. The flexible material used to construct the conduit sleeve 3351 may allow the conduit sleeve 3351 to further curve in order to correspond to the shape of the tubes 3350 (e.g., when worn by the patient).

In some forms, the conduit sleeve 3351 may include a first or superior opening 3352. The superior opening 3352 may be disposed at one end of the conduit sleeve 3351. The superior opening 3352 may be an opening to a passage that extends along at least a portion of the conduit sleeve 3351.

As shown in FIG. 4G, some forms of the conduit sleeve 3351 may also include an inferior extension 3354. The inferior extension 3354 may be positioned on an opposite end of the conduit sleeve 3351 from the superior opening 3352. The conduit sleeve 3351 may be customized to fit a particular user's face. For instance, the inferior extension 3354 of the conduit sleeve 3351 may be configured in a relatively more posterior region or anterior region of the patient's head.

Some forms of the inferior extension 3354 may include a rigid or semi-rigid piece (e.g., within the conduit sleeve 3351). The rigid or semi-rigid piece may be constructed from a plastic material, or a similar material. Alternatively, the inferior extension 3354 may be stiffened using a manufacturing process (e.g., stitching rigidised thread, flat knitting, using thicker material).

As shown in FIG. 4G, some forms of the inferior extension 3354 may include a connection member 3356. In the illustrated example, the connection member 3356 may be a magnet, although in other examples, the connection member 3356 may be a different type of connector (e.g., a mechanical fastener, an adhesive, hook and loop material, etc.). The connection member 3356 may also be positioned at an end of the inferior extension 3354, although the connection member 3356 could alternatively be positioned anywhere along the inferior extension 3354.

In some forms, the connection member 3356 (e.g., a magnet) may be removably connected to the magnets 3370-1 of the headgear 3302-1. For example, when the conduit sleeves 3351 are connected to the tubes 3350 (see e.g., FIG. 4J), the magnets 3370-1 connected to the inferior straps 3304-1 may be removably connected to the connection member 3356 in order to provide the tensile force.

### 5.3.5.2.1 Four-Point Arm Sleeve

As shown in FIG. 4H, another example of a sleeve is a four-point arm sleeve 3380, which may be usable with the rigidiser arms 3340 described above.

As shown in FIG. 4H, the four-point arm sleeve 3380 may include a curved shape that may be similar to the shape of the rigidiser arm 3340 shown in FIG. 4D. The flexible material used to construct the four-point arm sleeve 3380 may allow the four-point arm sleeve 3380 to further curve in order to correspond to the shape of the rigidiser arm 3340 (e.g., when worn by the patient and/or went bent by the patient).

As shown in FIG. 4H, some forms of the four-point arm sleeve 3380 may include an inferior extension 3384. The inferior extension 3384 may be positioned at an end of the four-point arm sleeve 3380.

In the illustrated example, the shape and/or structure of the inferior extension 3384 is substantially the same as the shape of the inferior extension 3354. For example, the inferior extension 3384 may be more rigid as compared to the rest of the four-point arm sleeve 3380 (e.g., as a result of rigidising thread or rigid material).

As shown in FIG. 4H, some forms of the inferior extension 3384 may include a connection member 3386. In the illustrated example, the connection member 3386 may be a magnet, although in other examples, the connection member 3386 may be a different type of connector (e.g., a mechanical fastener, an adhesive, hook and loop material, etc.). The connection member 3386 may also be positioned at an end of the inferior extension 3384, although the connection member 3386 could alternatively be positioned anywhere along the inferior extension 3384.

In some forms, the connection member 3386 (e.g., a magnet) may be removably connected to the magnets 3370-1 of the headgear 3302-1. For example, when the four-point arm sleeves 3380 are connected to the rigidiser arm 3340 (see e.g., FIG. 4K), the magnets 3370-1 connected to the inferior straps 3304-1 may be removably connected to the connection member 3386 in order to provide the tensile force.

As shown in FIG. 4H, the four-point arm sleeve 3380 may include a pair of tabs 3394, which may be similar to the tab 3320 on the tubes 3350. When the four-point arm sleeve 3380 is worn by the patient, the tabs 3394 may be positioned in substantially the same place on the patient's head as where the tabs 3320 are positioned when the patient wears the tubes 3350.

### 5.3.5.2.2 Two-Point Arm Sleeve

As shown in FIG. 4I, yet another example of a sleeve is a two-point arm sleeve 3380-1, which may be usable with the rigidiser arms 3340 described above.

In some forms, the two-point arm sleeve 3380-1 may be similar to the four-point arm sleeve 3380 described above. Only some similarities and differences may be described below.

As shown in FIG. 4I, the two-point arm sleeve 3380-1 may include an inferior opening 3388-1 that is positioned at an end of the two-point arm sleeve 3380-1. The inferior opening 3388-1 may form an opening to a passageway through the two-point arm sleeve 3380-1. In the illustrated example, the inferior opening 3388-1 may open into a surface of the conduit sleeve 3380-1.

As shown in FIG. 4I, the two-point arm sleeve 3380-1 may include a pair of tabs 3394-1, which may be similar to the tab 3320 on the tubes 3350. When the two-point arm sleeve 3380-1 is worn by the patient, the tabs 3394-1 may be positioned in substantially the same place on the patient's head as where the tabs 3320 are positioned when the patient wears the tubes 3350.

### 5.3.5.3 Assembled Patient Interfaces

As illustrated in FIGS. 4J to 4M, the various elements described above may be combined into four different patient interfaces. The different patient interfaces may allow patients to use different styles based on their individual comfort. The modularity of the different elements (e.g., the ability to be used in multiple styles of patient interfaces) may simplify manufacturing and/or may allow a patient to more easily switch between styles of patient interfaces.

### 5.3.5.3.1 Nose and Mouth Mask Tube Up Configuration

As illustrated in FIG. 4J, the patient may wear the cushion 3050-1 in a tube-up configuration with the tubes 3350 and the four-point headgear 3302-1. This assembly may form a tube up nose and mouth patient interface 3000-1.

In some forms, a conduit sleeve may be used with the tubes 3350 in order to enable a patient to experience the “tube up” air delivery style with the mouth and nose cushion 3050-1. As is described below, the conduit sleeve provides additional connection locations for connecting the four-point headgear 3302-1. However, other forms of connectors aside from or in addition to the conduit sleeve may be used.

In the illustrated example, the conduit sleeves may be connected to the tubes 3350 of the positioning and stabilising structure 3300. The tubes 3350 (via the conduit connection structure 3500), may be used to connect the tubes 3350 to the cushion 3050-1. The conduit sleeves provide the magnets in order to connect to the magnets 3370-1 (see e.g., FIG. 4E) of the four-point headgear 3302-1. Alternatively, a different connection form may be used.

As illustrated in FIG. 4J, the four-point headgear 3302-1 may connect in four separate locations in order to provide a tensile force that maintains the cushion 3050-1 in a sealing position on the patient's head.

For example, the inferior straps 3304-1 (e.g., via the magnetic members 3306-1) may removably connect to the magnets of the conduit sleeves. In use, each inferior strap 3304-1 may contact the patient's cheek (e.g., overlaying the masseter muscle). The inferior straps 3304-1 may also extend below the patient's ears.

### 5.3.5.3.2 Nose and Mouth Mask Tube Down Configuration

As illustrated in FIG. 4K, the patient may wear the cushion 3050-1 in a tube-down configuration with the rigidiser arms 3340 and the four-point headgear 3302-1. This assembly may form a tube down nose and mouth patient interface 3000-2.

In some forms, a conduit sleeve may be used with the rigidiser arms 3340 in order to enable a patient to experience the “tube down” air delivery style with the mouth and nose cushion 3050-1. As is described below, the conduit sleeve provides additional connection locations for connecting the four-point headgear 3302-1. However, other forms of connectors aside from or in addition to the conduit sleeve may be used.

In the illustrated example, the conduit sleeves may be connected to the rigidiser arms 3340 of the positioning and stabilising structure 3300. The rigidiser arms 3340 (via the conduit connection structure 3504), may be used to connect the rigidiser arms 3340 to the cushion 3050-1. The conduit sleeves provide the magnets in order to connect to the magnets 3370-1 (see e.g., FIG. 4E) of the four-point headgear 3302-1. Alternatively, a different connection form may be used.

As illustrated in FIG. 4K, the four-point headgear 3302-1 may connect in four separate locations in order to provide a tensile force that maintains the cushion 3050-1 in a sealing position on the patient's head.

For example, the inferior straps 3304-1 (e.g., via the magnetic members 3306-1) may removably connect to the magnets of the conduit sleeves. In use, each inferior strap 3304-1 may contact the patient's cheek (e.g., overlaying the masseter muscle). The inferior straps 3304-1 may also extend below the patient's ears.

### 5.3.5.3.3 Nose Mask Tube Up Configuration

As illustrated in FIG. 4L, the patient may wear the cushion 3050-2 in a tube-up configuration with the tubes 3350 and the two-point headgear 3302-2. This assembly may form a tube up nose only patient interface 3000-3

A conduit sleeve may be used with the tubes 3350, and may provide additional comfort to the patient. The sleeve may not add additional connection points to connect the positioning and stabilising structure 3300 on the cushion 3050-2. In the illustrated example, the tubes 3350 of the positioning and stabilising structure 3300 may be connected directly to the cushion 3050-2.

As illustrated in FIG. 4L, the two-point headgear 3302-2 may connect to the tabs 3320 on the tubes 3350 in order to provide a tensile force that maintains the cushion 3050-2 in a sealing position on the patient's head.

### 5.3.5.3.4 Nose Mask Tube Down Configuration

As illustrated in FIG. 4M, the patient may wear the cushion 3050-2 in a tube-up configuration with the rigidiser arms 3340 and the two-point headgear 3302-2. This assembly may form a tube down nose only patient interface 3000-4.

A conduit sleeve may be used with the rigidiser arms 3340, and may provide additional comfort to the patient. The sleeve may not add additional connection points to connect the positioning and stabilising structure 3300 on the cushion 3050-2. In the illustrated example, the rigidiser arms 3340 of the positioning and stabilising structure 3300 may be connected directly to the cushion 3050-2.

As illustrated in FIG. 4M, the two-point headgear 3302-2 may connect to the tabs 3320 on the sleeve in order to provide a tensile force that maintains the cushion 3050-2 in a sealing position on the patient's head.

### 5.3.5.3.5 Modularity of Elements

FIG. 4P illustrates how the different elements can be combined in order to form the four different patient interfaces described above. As illustrated, the different components may be reused for different styles of patient interfaces. This may allow for easier manufacturing and assembly, because a large number of the same components may be produced and used in a variety of styles. The only components not used in multiple styles may be the sleeves. However, the sleeves may be easier to manufacture. FIG. 4O shows a portion of air circuit 4170 that may interface with the patient interface, while FIG. 4N shows a vent housing 3404 that may interchangeably replace the air circuit shown in FIG. 4O, depending on the style of the patient interface.

### 5.4 RPT Device

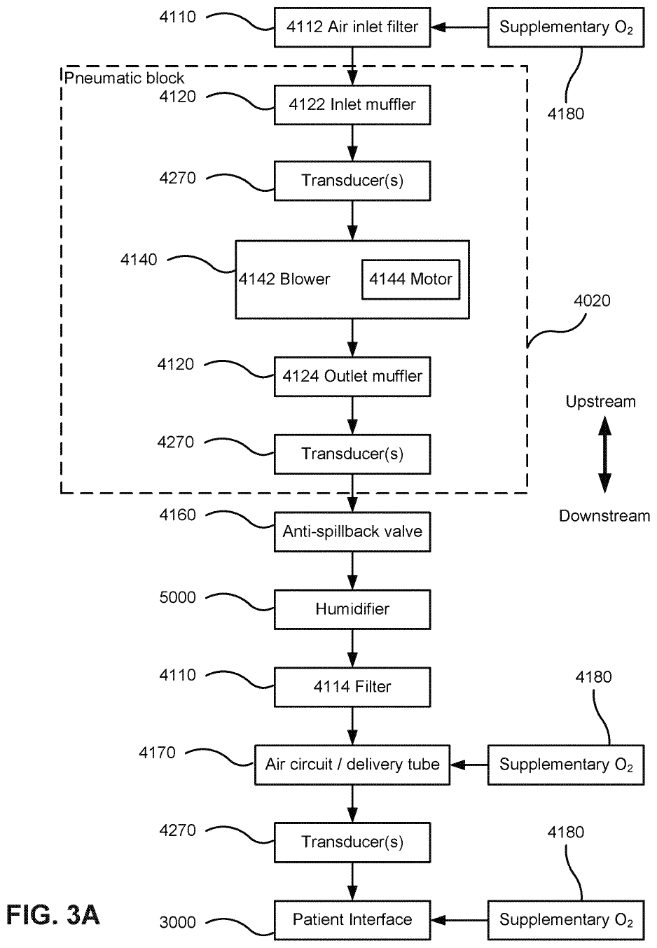

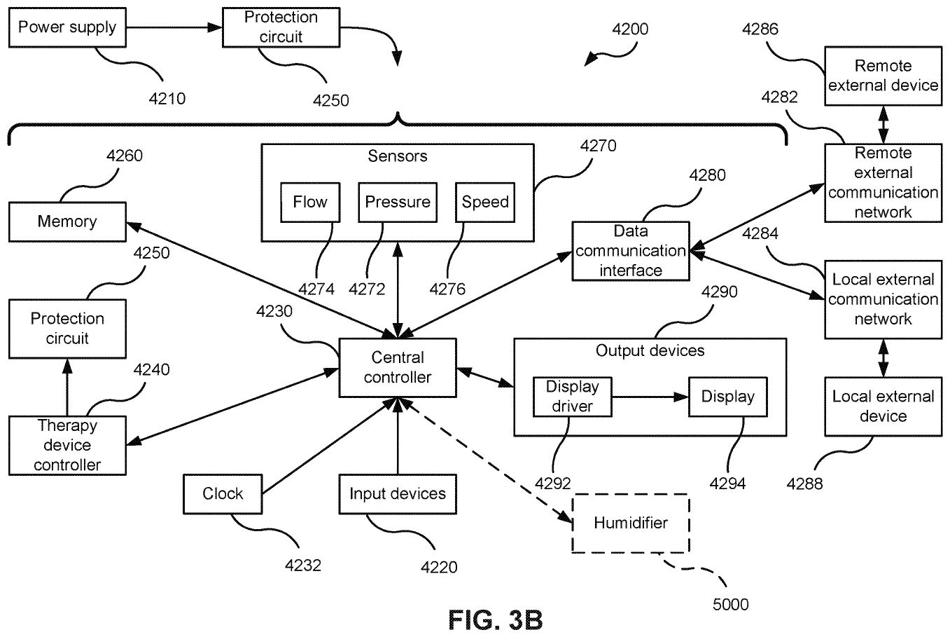

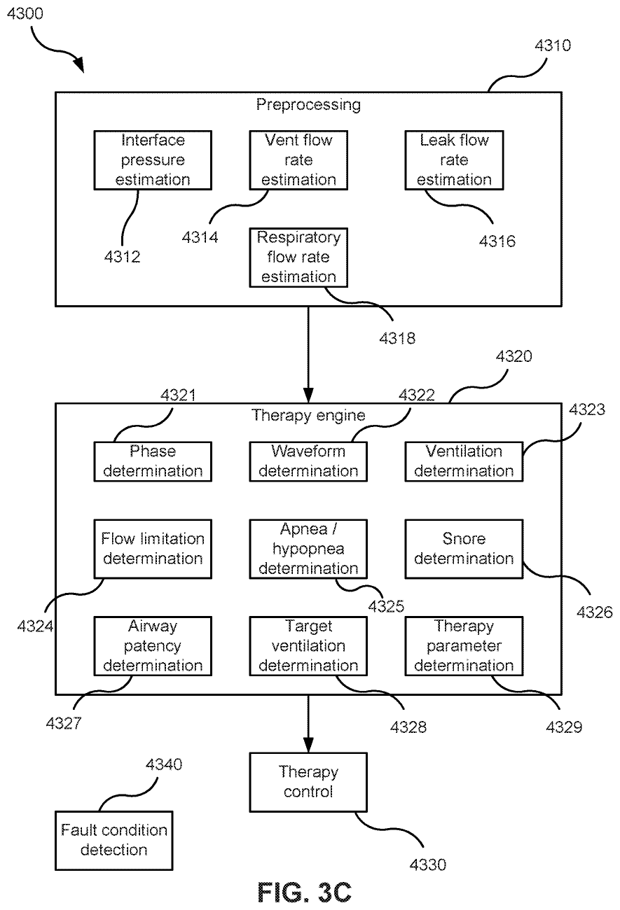

As shown in FIGS. 3A-3C, an RPT device 4000 in accordance with one aspect of the present technology comprises mechanical, pneumatic, and/or electrical components and is configured to execute one or more algorithms 4300, such as any of the methods, in whole or in part, described herein. The RPT device 4000 may be configured to generate a flow of air for delivery to a patient's airways, such as to treat one or more of the respiratory conditions described elsewhere in the present document.

### 5.4.1 Air Filter(s)

An RPT device in accordance with one form of the present technology may include an air filter 4110, or a plurality of air filters 4110.

In one form illustrated in FIG. 3A, an inlet air filter 4112 is located at the beginning of the pneumatic path upstream of a pressure generator 4140.

In one form illustrated in FIG. 3A, an outlet air filter 4114, for example an antibacterial filter, is located between an outlet of the pneumatic block 4020 and a patient interface 3000 or 3800.

### 5.4.2 Muffler(s)

An RPT device in accordance with one form of the present technology may include a muffler 4120, or a plurality of mufflers 4120.

In one form of the present technology (see e.g., FIG. 3A), an inlet muffler 4122 is located in the pneumatic path upstream of a pressure generator 4140.

In one form of the present technology, an outlet muffler 4124 is located in the pneumatic path between the pressure generator 4140 and a patient interface 3000 or 3800.

### 5.4.3 Pressure Generator

In one form of the present technology, a pressure generator 4140 for producing a flow, or a supply, of air at positive pressure is a controllable blower 4142. For example, the blower 4142 may include a brushless DC motor 4144 with one or more impellers. The impellers may be located in a volute. The blower may be capable of delivering a supply of air, for example at a rate of up to about 120 litres/minute, at a positive pressure in a range from about 4 cmH2O to about 20 cmH2O, or in other forms up to about 30 cmH2O when delivering respiratory pressure therapy. The blower may be as described in any one of the following patents or patent applications the contents of which are incorporated herein by reference in their entirety: U.S. Pat. Nos. 7,866,944; 8,638,014; 8,636,479; and PCT Patent Application Publication No. WO 2013/020167.

The pressure generator 4140 may be under the control of the therapy device controller 4240.

In other forms, a pressure generator 4140 may be a piston-driven pump, a pressure regulator connected to a high pressure source (e.g. compressed air reservoir), or a bellows.

### 5.4.4 Transducer(s)

Transducers may be internal of the RPT device, or external of the RPT device. External transducers may be located for example on or form part of the air circuit, e.g., the patient interface. External transducers may be in the form of non-contact sensors such as a Doppler radar movement sensor that transmit or transfer data to the RPT device.

In one form of the present technology (see e.g., FIG. 3A), one or more transducers 4270 are located upstream and/or downstream of the pressure generator 4140.

The one or more transducers 4270 may be constructed and arranged to generate signals representing properties of the flow of air such as a flow rate, a pressure or a temperature at that point in the pneumatic path.

In one form of the present technology, one or more transducers 4270 may be located proximate to the patient interface 3000 or 3800.

In one form, a signal from a transducer 4270 may be filtered, such as by low-pass, high-pass or band-pass filtering.

### 5.4.5 RPT Sensors

### 5.4.5.1 Flow Rate Sensor

A flow rate sensor 4274 in accordance with the present technology may be based on a differential pressure transducer, for example, an SDP600 Series differential pressure transducer from SENSIRION.

In one form, a signal generated by the flow rate sensor 4274 and representing a flow rate is received by the central controller 4230.

### 5.4.5.2 Pressure Sensor

A pressure sensor 4272 in accordance with the present technology is located in fluid communication with the pneumatic path. An example of a suitable pressure sensor is a transducer from the HONEYWELL ASDX series. An alternative suitable pressure sensor is a transducer from the NPA Series from GENERAL ELECTRIC.

In one form, a signal generated by the pressure sensor 4272 and representing a pressure is received by the central controller 4230.

### 5.4.6 Motor Speed Transducer

In one form of the present technology a motor speed transducer 4276 is used to determine a rotational velocity of the motor 4144 and/or the blower 4142. A motor speed signal from the motor speed transducer 4276 may be provided to the therapy device controller 4240. The motor speed transducer 4276 may, for example, be a speed sensor, such as a Hall effect sensor.

### 5.4.7 Anti-Spill Back Valve

As shown in FIG. 3A, one form of the present technology, an anti-spill back valve 4160 is located between the humidifier 5000 and the pneumatic block 4020. The anti-spill back valve is constructed and arranged to reduce the risk that water will flow upstream from the humidifier 5000, for example to the motor 4144.

### 5.4.8 RPT Device Electrical Components

### 5.4.8.1 Power Supply

A power supply 4210 may be located internal or external of the external housing 4010 of the RPT device 4000.

In one form of the present technology, power supply 4210 provides electrical power to the RPT device 4000 only. In another form of the present technology, power supply 4210 provides electrical power to both RPT device 4000 and humidifier 5000.

### 5.4.8.2 Input Devices

In one form of the present technology, an RPT device 4000 includes one or more input devices 4220 in the form of buttons, switches or dials to allow a person to interact with the device. The buttons, switches or dials may be physical devices, or software devices accessible via a touch screen. The buttons, switches or dials may, in one form, be physically connected to the external housing 4010, or may, in another form, be in wireless communication with a receiver that is in electrical connection to the central controller 4230.

In one form, the input device 4220 may be constructed and arranged to allow a person to select a value and/or a menu option.

### 5.4.8.3 Central Controller

In one form of the present technology, the central controller 4230 is one or a plurality of processors suitable to control an RPT device 4000. The central controller 4230 is shown in FIG. 3B.

Suitable processors may include an x86 INTEL processor, a processor based on ARM® Cortex®-M processor from ARM Holdings such as an STM32 series microcontroller from ST MICROELECTRONIC. In certain alternative forms of the present technology, a 32-bit RISC CPU, such as an STR9 series microcontroller from ST MICROELECTRONICS or a 16-bit RISC CPU such as a processor from the MSP430 family of microcontrollers, manufactured by TEXAS INSTRUMENTS may also be suitable.

In one form of the present technology, the central controller 4230 is a dedicated electronic circuit.

In one form, the central controller 4230 is an application-specific integrated circuit. In another form, the central controller 4230 comprises discrete electronic components.

The central controller 4230 may be configured to receive input signal(s) from one or more transducers 4270, one or more input devices 4220, and/or the humidifier 5000.

The central controller 4230 may be configured to provide output signal(s) to one or more of an output device 4290, a pressure generator 4140, a therapy device controller 4240, a data communication interface 4280, and/or the humidifier 5000.

In some forms of the present technology, the central controller 4230 is configured to implement the one or more methodologies described herein, such as the one or more algorithms 4300 which may be implemented with processor-control instructions, expressed as computer programs stored in a non-transitory computer readable storage medium, such as memory 4260. In some forms of the present technology, the central controller 4230 may be integrated with an RPT device 4000. However, in some forms of the present technology, some methodologies may be performed by a remotely located device. For example, the remotely located device may determine control settings for a ventilator or detect respiratory related events by analysis of stored data such as from any of the sensors described herein.

### 5.4.8.4 Clock

The RPT device 4000 may include a clock 4232 that is connected to the central controller 4230.

### 5.4.8.5 Therapy Device Controller

In one form of the present technology, therapy device controller 4240 is a therapy control module 4330 that forms part of the algorithms 4300 executed by the central controller 4230.

In one form of the present technology, therapy device controller 4240 is a dedicated motor control integrated circuit. For example, in one form a MC33035 brushless DC motor controller, manufactured by ONSEMI is used.

### 5.4.8.6 Protection Circuits

The one or more protection circuits 4250 in accordance with the present technology may comprise an electrical protection circuit, a temperature and/or pressure safety circuit.

### 5.4.8.7 Memory

In accordance with one form of the present technology the RPT device 4000 includes memory 4260, e.g., non-volatile memory. In some forms, memory 4260 may include battery powered static RAM. In some forms, memory 4260 may include volatile RAM.

Memory 4260 may be located on the PCBA 4202. Memory 4260 may be in the form of EEPROM, or NAND flash.

Additionally, or alternatively, RPT device 4000 includes a removable form of memory 4260, for example a memory card made in accordance with the Secure Digital (SD) standard.

In one form of the present technology, the memory 4260 acts as a non-transitory computer readable storage medium on which is stored computer program instructions expressing the one or more methodologies described herein, such as the one or more algorithms 4300.

### 5.4.8.8 Data Communication Systems

In one form of the present technology, a data communication interface 4280 is provided, and is connected to the central controller 4230 (see e.g., FIG. 3B). Data communication interface 4280 may be connectable to a remote external communication network 4282 and/or a local external communication network 4284. The remote external communication network 4282 may be connectable to a remote external device 4286. The local external communication network 4284 may be connectable to a local external device 4288.

In one form, data communication interface 4280 is part of the central controller 4230. In another form, data communication interface 4280 is separate from the central controller 4230, and may comprise an integrated circuit or a processor.

In one form, remote external communication network 4282 is the Internet. The data communication interface 4280 may use wired communication (e.g. via Ethernet, or optical fibre) or a wireless protocol (e.g. CDMA, GSM, LTE) to connect to the Internet.

In one form, local external communication network 4284 utilises one or more communication standards, such as Bluetooth, or a consumer infrared protocol.

In one form, remote external device 4286 is one or more computers, for example a cluster of networked computers. In one form, remote external device 4286 may be virtual computers, rather than physical computers. In either case, such a remote external device 4286 may be accessible to an appropriately authorised person such as a clinician.

The local external device 4288 may be a personal computer, mobile phone, tablet or remote control.

### 5.4.8.9 Output Devices Including Optional Display, Alarms

An output device 4290 in accordance with the present technology may take the form of one or more of a visual, audio and haptic unit. A visual display may be a Liquid Crystal Display (LCD) or Light Emitting Diode (LED) display.

### 5.4.8.9.1 Display Driver

A display driver 4292 receives as an input the characters, symbols, or images intended for display on the display 4294, and converts them to commands that cause the display 4294 to display those characters, symbols, or images.

### 5.4.8.9.2 Display

A display 4294 is configured to visually display characters, symbols, or images in response to commands received from the display driver 4292. For example, the display 4294 may be an eight-segment display, in which case the display driver 4292 converts each character or symbol, such as the figure “0”, to eight logical signals indicating whether the eight respective segments are to be activated to display a particular character or symbol.

Referring now to FIGS. 3A-3E, in some forms of the present technology, the central controller 4230 may be configured to implement one or more algorithms 4300 expressed as computer programs stored in a non-transitory computer readable storage medium, such as memory 4260. The algorithms 4300 are generally grouped into groups referred to as modules.

In other forms of the present technology, some portion or all of the algorithms 4300 may be implemented by a controller of an external device such as the local external device 4288 or the remote external device 4286. In such forms, data representing the input signals and/or intermediate algorithm outputs necessary for the portion of the algorithms 4300 to be executed at the external device may be communicated to the external device via the local external communication network 4284 or the remote external communication network 4282. In such forms, the portion of the algorithms 4300 to be executed at the external device may be expressed as computer programs, such as with processor control instructions to be executed by one or more processor(s), stored in a non-transitory computer readable storage medium accessible to the controller of the external device. Such programs configure the controller of the external device to execute the portion of the algorithms 4300.

In such forms, the therapy parameters generated by the external device via the therapy engine module 4320 (if such forms part of the portion of the algorithms 4300 executed by the external device) may be communicated to the central controller 4230 to be passed to the therapy control module 4330.

### 5.4.8.10 Pre-Processing Module

A pre-processing module 4310 in accordance with one form of the present technology receives as an input a signal from a transducer 4270, for example a flow rate sensor 4274 or pressure sensor 4272, and performs one or more process steps to calculate one or more output values that will be used as an input to another module, for example a therapy engine module 4320.

In one form of the present technology, the output values include the interface pressure Pm, the vent flow rate Qv, the respiratory flow rate Qr, and the leak flow rate Ql.

In various forms of the present technology, the pre-processing module 4310 comprises one or more of the following algorithms: interface pressure estimation algorithm 4312, vent flow rate estimation algorithm 4314, leak flow rate estimation algorithm 4316, and respiratory flow rate estimation algorithm 4318.

### 5.4.8.10.1 Interface Pressure Estimation

In one form of the present technology, an interface pressure estimation algorithm 4312 receives as inputs a signal from the pressure sensor 4272 indicative of the pressure in the pneumatic path proximal to an outlet of the pneumatic block (the device pressure Pd) and a signal from the flow rate sensor 4274 representative of the flow rate of the airflow leaving the RPT device 4000 (the device flow rate Qd). The device flow rate Qd, absent any supplementary gas 4192, may be used as the total flow rate Qt. The interface pressure estimation algorithm 4312 estimates the pressure drop ΔP through the air circuit 4170. The dependence of the pressure drop ΔP on the total flow rate Qt may be modelled for the particular air circuit 4170 by a pressure drop characteristic ΔP(Q). The interface pressure estimation algorithm, 4312 then provides as an output an estimated pressure, Pm, in the patient interface 3000 or 3800. The pressure, Pm, in the patient interface 3000 or 3800 may be estimated as the device pressure Pd minus the air circuit pressure drop ΔP.

### 5.4.8.10.2 Vent Flow Rate Estimation

In one form of the present technology, a vent flow rate estimation algorithm 4314 receives as an input an estimated pressure, Pm, in the patient interface 3000 or 3800 from the interface pressure estimation algorithm 4312 and estimates a vent flow rate of air, Qv, from a vent 3400 in a patient interface 3000 or 3800. The dependence of the vent flow rate Qv on the interface pressure Pm for the particular vent 3400 in use may be modelled by a vent characteristic Qv(Pm).

### 5.4.8.10.3 Leak Flow Rate Estimation

In one form of the present technology, a leak flow rate estimation algorithm 4316 receives as an input a total flow rate, Qt, and a vent flow rate Qv, and provides as an output an estimate of the leak flow rate Ql. In one form, the leak flow rate estimation algorithm estimates the leak flow rate Ql by calculating an average of the difference between total flow rate Qt and vent flow rate Qv over a period sufficiently long to include several breathing cycles, e.g. about 10 seconds.

In one form, the leak flow rate estimation algorithm 4316 receives as an input a total flow rate Qt, a vent flow rate Qv, and an estimated pressure, Pm, in the patient interface 3000 or 3800, and provides as an output a leak flow rate Ql, by calculating a leak conductance, and determining a leak flow rate Ql to be a function of leak conductance and pressure, Pm. Leak conductance is calculated as the quotient of low pass filtered non-vent flow rate equal to the difference between total flow rate Qt and vent flow rate Qv, and low pass filtered square root of pressure Pm, where the low pass filter time constant has a value sufficiently long to include several breathing cycles, e.g. about 10 seconds. The leak flow rate Ql may be estimated as the product of leak conductance and a function of pressure, Pm.

### 5.4.8.10.4 Respiratory Flow Rate Estimation

In one form of the present technology, a respiratory flow rate estimation algorithm 4318 receives as an input a total flow rate, Qt, a vent flow rate, Qv, and a leak flow rate, Ql, and estimates a respiratory flow rate of air, Qr, to the patient, by subtracting the vent flow rate Qv and the leak flow rate Ql from the total flow rate Qt.

### 5.4.8.11 Therapy Engine Module

In one form of the present technology, a therapy engine module 4320 receives as inputs one or more of a pressure, Pm, in a patient interface 3000 or 3800, and a respiratory flow rate of air to a patient, Qr, and provides as an output one or more therapy parameters.

In one form of the present technology, a therapy parameter is a treatment pressure Pt.

In one form of the present technology, therapy parameters are one or more of an amplitude of a pressure variation, a base pressure, and a target ventilation.

In various forms, the therapy engine module 4320 comprises one or more of the following algorithms: phase determination 4321, waveform determination 4322, ventilation determination 4323, inspiratory flow limitation determination 4324, apnea/hypopnea determination 4325, snore determination 4326, airway patency determination 4327, target ventilation determination 4328, and therapy parameter determination 4329.

### 5.4.8.11.1 Phase Determination

In one form of the present technology, the RPT device 4000 does not determine phase.

In one form of the present technology, a phase determination algorithm 4321 receives as an input a signal indicative of respiratory flow rate, Qr, and provides as an output a phase Φ of a current breathing cycle of a patient 1000.

### 5.4.8.11.2 Waveform Determination

In one form of the present technology, the therapy parameter determination algorithm 4329 provides an approximately constant treatment pressure throughout a respiratory cycle of a patient.

In other forms of the present technology, the therapy control module 4330 controls the pressure generator 4140 to provide a treatment pressure Pt that varies as a function of phase Φ of a respiratory cycle of a patient according to a waveform template Π(Φ).

In one form of the present technology, a waveform determination algorithm 4322 provides a waveform template Π(Φ) with values in the range [0, 1] on the domain of phase values Φ provided by the phase determination algorithm 4321 to be used by the therapy parameter determination algorithm 4329.

### 5.4.8.11.3 Ventilation Determination

In one form of the present technology, a ventilation determination algorithm 4323 receives an input a respiratory flow rate Qr, and determines a measure indicative of current patient ventilation, Vent.

### 5.4.8.11.4 Determination of Inspiratory Flow Limitation

In one form of the present technology, the central controller 4230 executes an inspiratory flow limitation determination algorithm 4324 for the determination of the extent of inspiratory flow limitation.

### 5.4.8.11.5 Determination of Apneas and Hypopneas

In one form of the present technology, the central controller 4230 executes an apnea/hypopnea determination algorithm 4325 for the determination of the presence of apneas and/or hypopneas.

### 5.4.8.11.6 Determination of Snore

In one form of the present technology, the central controller 4230 executes one or more snore determination algorithms 4326 for the determination of the extent of snore.

### 5.4.8.11.7 Determination of Airway Patency

In one form of the present technology, the central controller 4230 executes one or more airway patency determination algorithms 4327 for the determination of the extent of airway patency.

### 5.4.8.11.8 Determination of Target Ventilation

In one form of the present technology, the central controller 4230 takes as input the measure of current ventilation, Vent, and executes one or more target ventilation determination algorithms 4328 for the determination of a target value Vtgt for the measure of ventilation.

### 5.4.8.11.9 Determination of Therapy Parameters

In some forms of the present technology, the central controller 4230 executes one or more therapy parameter determination algorithms 4329 for the determination of one or more therapy parameters using the values returned by one or more of the other algorithms in the therapy engine module 4320.

### 5.4.8.12 Therapy Control Module

The therapy control module 4330 in accordance with one aspect of the present technology receives as inputs the therapy parameters from the therapy parameter determination algorithm 4329 of the therapy engine module 4320, and controls the pressure generator 4140 to deliver a flow of air in accordance with the therapy parameters.

In one form of the present technology, the therapy parameter is a treatment pressure Pt, and the therapy control module 4330 controls the pressure generator 4140 to deliver a flow of air whose interface pressure Pm at the patient interface 3000 or 3800 is equal to the treatment pressure Pt.

### 5.4.8.13 Detection of Fault Conditions

In one form of the present technology, the central controller 4230 executes one or more methods 4340 for the detection of fault conditions. The fault conditions detected by the one or more methods 4340 may include at least one of the following:
Power failure (no power, or insufficient power)
Transducer fault detection
Failure to detect the presence of a component
Operating parameters outside recommended ranges (e.g. pressure, flow rate, temperature, PaO2)
Failure of a test alarm to generate a detectable alarm signal.

Upon detection of the fault condition, the corresponding algorithm signals the presence of the fault by one or more of the following:
Initiation of an audible, visual &/or kinetic (e.g. vibrating) alarm
Sending a message to an external device
Logging of the incident

### 5.5 Patient Interface Detection

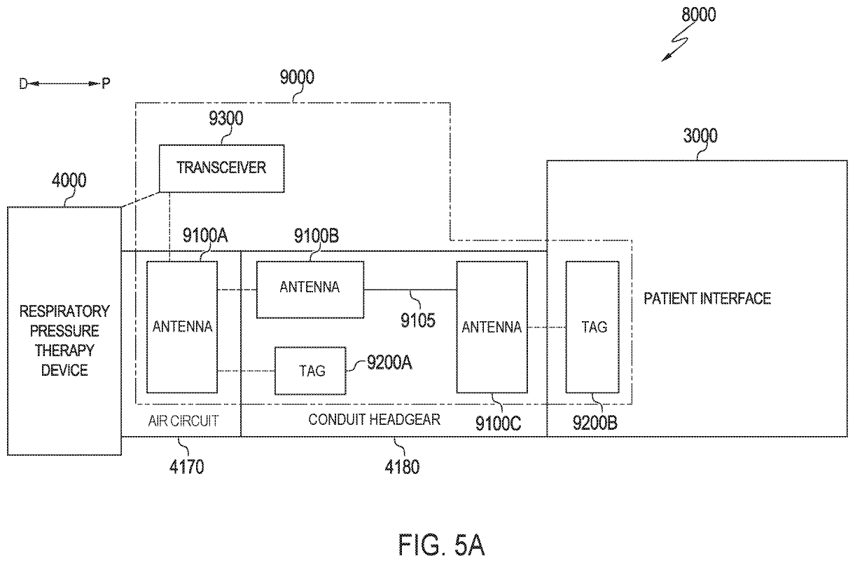

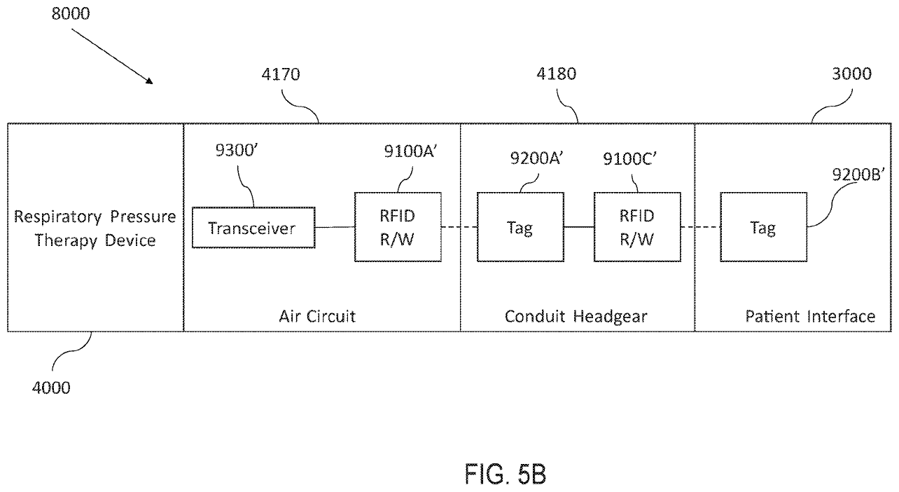

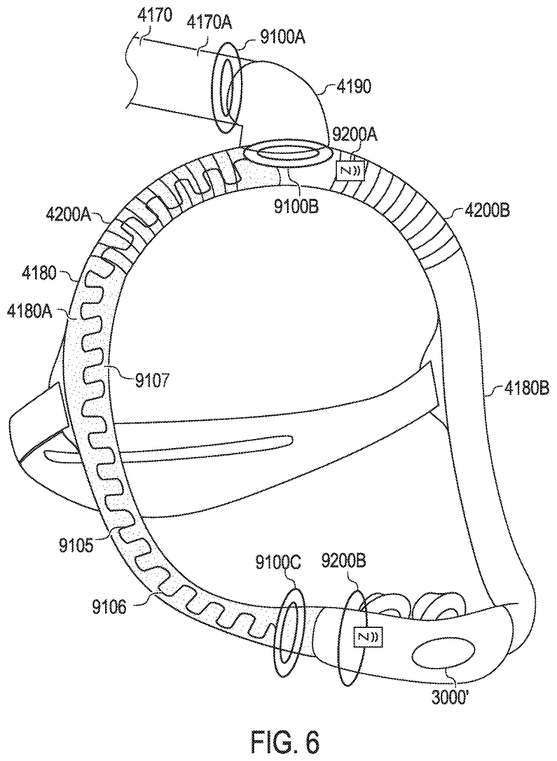

FIG. 5A illustrates a schematic view of a respiratory therapy system 8000, and FIG. 6 illustrates an example of the technology described herein in the context of a tube-up patient interface. FIG. 5B illustrates another schematic view of the respiratory therapy system 8000. The respiratory therapy system 8000 may be configured to wirelessly detect the identification of a patient interface 3000 or an accessory that a patient is using during a therapy session. In particular, the respiratory therapy system 8000 may be configured for use with a tube-up patient interface, such as patient interface 3000-1 of FIG. 4J or patient interface 3000-3 of FIG. 4L, e.g., a full-face, nasal, or pillow cushion tube-up patient interface. The tube-up patient interface 3000 may include one or more Radio Frequency Identification (RFID) tags incorporated within the patient interface 3000. For example, an RFID tag may be incorporated with one or more of a pillow cushion or other seal-forming structure 3100 of patient interface 3000, a conduit of the patient interface 3000 (such as the tubes 3350), or near a connection point with an air circuit. Aspects of the technology described herein may relate to the inclusion of extended antenna portions within a tube-up patient interface having stretchable portions, e.g., corrugated concertina portions such as the extendable concertina structure 3362, described above, in order to facilitate accurate reading of the one or more RFID tags even when positioned further away from a reader antenna and transceiver. Although the technology disclosed in this section is discussed in regards to a tube-up configuration, the technology may also be used with tube-down configurations (such as patient interfaces 3000-2 of FIG. 4K or 3000-4 of FIG. 4M).

The respiratory therapy system 8000 includes a patient interface 3000 and the respiratory pressure therapy (RPT) device 4000 configured to be fluidly coupled by the air circuit 4170, which may be a conduit or tube, as discussed above. Respiratory therapy system 8000 further includes the conduit headgear 4180 configured to fluidly connect with the air circuit 4170 and direct a flow of gas from the RPT device 4000, through the air circuit 4170, through the conduit headgear 4180, and to the patient interface 3000. The RPT device 4000 is configured to supply a flow of gas, for example, air, which may be supplemented with oxygen, through the air circuit 4170 and the conduit headgear 4180 to the patient interface 3000. Although not shown, the respiratory therapy system 8000 may further comprise a humidifier, an oxygen source, and/or data management systems, as discussed above, with respect to the RPT device 4000.

The RPT device 4000 may be used individually or as part of the system 8000 to deliver one or more of a number of therapies described above, such as by operating the device to generate a flow of air for delivery to an interface (e.g., the patient interface 3000) to the airways of the user. The flow of air may be pressure-controlled (for respiratory pressure therapies) or flow-controlled (for flow therapies such as HFT). Thus, the RPT device 4000 may also act as a flow therapy device. The RPT device 4000 may, for example, include a CPAP device and a ventilator.

As described above, the respiratory therapy system 8000 may further comprise a Radio Frequency Identification (RFID) system 9000. RFID is a form of wireless communication that incorporates the use of electromagnetic or electrostatic coupling in the radio frequency portion of the electromagnetic spectrum, for example, to uniquely identify and/or track an object. The RFID system 9000 may operate according to the principle of inductive coupling.

An example RFID system (such as RFID system 9000) may include at least one RFID reader and at least one RFID tag. An RFID reader may include an antenna (sometimes called a “reader antenna” herein) and a RFID reader and/or writer (sometimes called a “reader chip” herein), which is a type of integrated circuit. An illustrative example of a RFID reader may include, for example, the MFRC522 from NXP. In an example, the RFID reader can be coupled to, for example, a microcontroller (e.g., a hardware processor) that is used to read and/or process the data that is communicated to/from the RFID reader. An RFID tag may include an antenna (sometimes called a “tag antenna” herein) and another integrated circuit (sometimes called a “tag chip” herein), which may include non-transitory memory for storing data therein.

The RFID system 9000 may, for example, be configured to detect one or more characteristics of the respiratory therapy being delivered from the RPT device 4000 to the patient interface 3000, the identification of the patient interface 3000, the identification of the air circuit 4170, the identification of the conduit headgear 4180, and/or the identification of an accessory device (not shown) directly or indirectly coupled to the respiratory therapy system 8000. Exemplary accessory devices may include, but are not limited to, a mask headgear, a cushion or other seal-forming structure 3100 on the patient interface 3000, an air filter, a humidifier, one or more components of a humidification system (e.g., heat and moisture exchangers or waterless humidifiers), an additional conduit, and/or a dongle accessory.

In one aspect, an RFID reader, e.g., a first antenna 9100A and a transceiver 9300, which may be a reader chip, may be associated with the air circuit 4170, as described further below. Any of the antennas disclosed herein, including the first antenna 9100A, may include a flexible printed circuit, a coil wire, and/or conductive printing on a substrate, such as plastic. Additionally, or alternatively, the first transceiver 9300 may be associated with (e.g., disposed in or on) the RPT device 4000. When the air circuit 4170 is coupled to the conduit headgear 4180 in a tube-up configuration, an RFID tag 9200 associated, e.g., with the patient interface 3000 or a cushion or other seal-forming structure 3100, may be spaced apart from the first antenna 9100A and the transceiver 9300. As a result, an RFID tag 9200 associated with, e.g., the patient interface 3000 or a cushion or other seal-forming structure 3100, may be out of reading range of the first antenna 9100A and the transceiver 9300. While the reading range may be increased by increasing a size of the antenna associated with the RFID tag 9200, the size of the conduit headgear 4180, the patient interface 3000, one or more cushions or other seal-forming structures 3100, or other accessories may be relatively small. Accordingly, it may not be possible to accommodate a larger antenna as part of the conduit headgear 4180, the patient interface 3000, one or more cushions or other seal-forming structures 3100, or other accessories to increase the reading range.

As an example, the cushion for a typical tube-up patient interface (e.g., a full-face, nasal, or pillow cushion tube-up configuration) may be about 250 mm to about 300 mm away from a proximal end of the air circuit 4170 when the air circuit 4170 is coupled to the conduit headgear 4180. It thus may be difficult for the first antenna 9100A associated with the air circuit 4170 to detect an RFID tag associated with the cushion. Further, with a smaller-diameter air circuit 4170, there may not be room to accommodate a sufficiently large first antenna 9100A on or within the air circuit 4170.

In light of the above, aspects of the technology described herein are drawn to an extended antenna configuration to create a medium to transfer the required energy from an RFID reader (e.g., the antenna 9100A and the transceiver 9300) to the RFID tag 9200 at a distance greater, e.g., than the magnetic field emitted by the antenna 9100A in which the RFID system 9000 is configured to operate. Further, aspects of the extended antenna technology described herein may be compatible for use with stretchable or the flexible conduit headgear 4180, which may include, e.g., a stretchable corrugated concertina portion along both tubes of the conduit headgear.

Aspects of the technology described herein are also drawn to providing power wirelessly from, for example, an antenna to electrical components (e.g., 9200A, 9200B, 9100B, 9100C, etc.) that are included in flexible conduit headgear (e.g., 4180) and/or a patient interface (e.g., 3000). In an aspect, energy provided in this manner may allow for reception of wireless power at one end of the flexible conduit headgear and then provided, via a wire included with the flexible conduit headgear, to other electrical components of the flexible conduit headgear. In an example, another antenna (e.g., 9100C) in the flexible conduit headgear may be used to wirelessly provide power to another electrical component, such as a tag included in a patient interface. Accordingly, power may be supplied to electrical components of the flexible conduit headgear (and patient interface in certain examples) without having to rely upon or use a physical electrical connection between, for example, the flexible conduit headgear and another component, such as air circuit 4170. Furthermore, as power is supplied wirelessly to the flexible conduit headgear, there is no need to include a power source (e.g., a battery) within the flexible conduit headgear.

The RFID system 9000 includes a first antenna 9100A, a second antenna 9100B, a third antenna 9100C, a first transponder 9200A (hereinafter referred to as the “first tag”), a second transponder 9200B (hereinafter referred to as the “second tag”), and a transceiver 9300. Each of the first antenna 9100A, the second antenna 9100B, and the third antenna 9100C is configured to emit radio waves and receive reflected signals, for example, from the first tag 9200A and/or the second tag 9200B. Each of the first antenna 9100A, the second antenna 9100B, and the third antenna 9100C may be a linear antenna, and thus may emit a linear polarized signal, or each of the first antenna 9100A, the second antenna 9100B, and the third antenna 9100C may be a circular antenna, and thus may emit a circularly polarized signal. A combination of linear antennas and circular antennas are also contemplated.

The first antenna 9100A may be disposed at a proximal portion of the air circuit 4170 at a region where the air circuit 4170 is configured to couple with the conduit headgear 4180. For example, the first antenna 9100A may be disposed at a portion of the air circuit 4170, such as, for example, within a lumen of the air circuit 4170, within a cover on a proximal portion of the air circuit 4170, within walls of the air circuit 4170, or on an external surface of the air circuit 4170. In some aspects, the first antenna 9100A may be incorporated as part of an adapter (not shown) configured to couple the air circuit 4170 with conduit headgear 4180. The first antenna 9100A and the transceiver 9300 may also be configured to transmit the data received from the first tag 9200A and/or the second antenna 9100B, for example, to another component within the system 8000, e.g., the RPT device 4000 or a controller (such as the central controller 4230), or to a component external to the system 8000 (such as the remote external device 4286 or the local external device 4288).

In an example, the transmission of such data may be carried out using an appropriate transceiver, which may, as noted above, the same as transceiver 9300 or may be separate/different from transceiver 9300 (e.g., different from the transceiver of the RFID reader). For example, as shown in FIG. 5B, transceiver 9300′ may communicate data to/from RFID reader 9100A′. In other words, in an example, the air circuit 4170 may include a first transceiver (which may be part of an RFID reader) for carrying out RFID communication (via antenna 9100A) with an RFID tag included with conduit headgear 4180 and a second, separate, transceiver (9300′) for communicating data to/from, for example, RPT device 4000 or another device, such as a smartphone or other remote computing device.

The second antenna 9100B may be associated with the conduit headgear 4180. For example, the second antenna 9100B may be located at a distal portion of the conduit headgear 4180 (e.g., near where the proximal portion of the air circuit 4170 is configured to couple with conduit headgear 4180). For example, the second antenna 9100B may be disposed on or within a portion of the conduit headgear 4180 (e.g., within a cover on a distal portion of the conduit headgear 4180, on an external surface of conduit headgear 4180, or within walls of conduit headgear 4180). The second antenna 9100B is configured to transmit data to the first antenna 9100A and the transceiver 9300. The second antenna 9100B is physically and/or electrically coupled to the third antenna 9100C, for example, via one or more electrical connections 9105. Electrical connections 9105 may include any type of conduit for transmitting electrical signals, such as one or more of a wire, a flexible printed circuit (FPC), a conductive textile, a conductive printed plastic, or conductive silicon. Accordingly, data may be transmitted from the third antenna 9100C, which may be spaced apart from the second antenna 9100B, to the second antenna 9100B via electrical connection(s) 9105. The electrical connection(s) 9105 may be disposed on or within walls of the conduit headgear 4180. For example, the electrical connection(s) 9105 may be disposed within a material comprising the conduit headgear 4180 or external to the conduit headgear 4180. In some configurations, the electrical connection(s) 9105 may be at least partially coated or sheathed, for example, to prevent breakdown due to exposure to fluid, humidity, or temperatures.

In some aspects, the electrical connection(s) 9105 may not be located in an air passageway of the conduit headgear 4180. Locating the electrical connection(s) 9105 within the conduit headgear 4180 in the flow of gas may disrupt the flow of gas, which may interfere with treatment. Alternatively or additionally, the locating electrical connection(s) 9105 within the conduit headgear 4180 may expose the electrical connection(s) 9105 to moisture in the humidified air flowing through, which may affect the integrity of the electrical connection(s) 9105, e.g., leading to corrosion of the wires. If electrical connection(s) 9105 are located in an air passageway, they may be encased, sheathed, covered, or otherwise protected to inhibit exposure to moisture.

The third antenna 9100C may be disposed at a proximal portion of the conduit headgear 4180 (e.g., near the patient interface 3000). For example, the third antenna 9100C may be on or within a portion of the conduit headgear 4180 (e.g., within a cover on a distal portion of conduit the headgear 4180, or on an external surface of the conduit headgear 4180). The third antenna 9100C may be configured to transmit a signal to the second antenna 9100B, for example, via the electrical connection(s) 9105. The third antenna 9100C may also be configured to receive a signal from the second tag 9200B, which may be transmitted to the second antenna 9100B. The second antenna 9100B may then be read by the first antenna 9100A.

As described above, the RFID system 9000 may include at least one or more tags 9200. In the examples shown in FIGS. 5A, 5B and 6, the RFID system 9000 may comprise the first tag 9200A and the second tag 9200B, which may each be configured to emit radio waves to transmit information. The first tag 9200A and the second tag 9200B may each contain a microchip configured to store and process information. For example, the first tag 9200A may store data related to a unique identifier of the first tag 9200A and the second antenna 9100B to enable the first tag 9200A to receive and/or transmit radio signals to the second antenna 9100B. Similarly, the second tag 9200B may store data related to the unique identifier of the second tag 9200B and the third antenna 9100C to enable the second tag 9200B to receive and/or transmit radio signals to the third antenna 9100C. The first tag 9200A and the second tag 9200B may be passive tags. If a passive tag is used, the passive tag may rely on the power of the antenna to transmit data, and thus may have a shorter transmittal range. For example, the first tag 9200A may receive all of the required energy from a magnetic field in which first tag 9200A operates. Similarly, the second tag 9200B may receive all of the required energy from a magnetic field in which the second tag 9200B operates.

In an example, a battery (not shown) may be included in the conduit headgear 4180 to supply power to any or all of antenna 9100B, tag 9200A, and antenna 9100C. A battery may be included in the first tag 9200A, the second tag 9200B (e.g., such that they are active tags), or be provided separately. In an example, the battery life of the first tag 9200A, second tag 9200B, and/or an included battery, may have a life span designed to match or exceed the service life of the associated component (e.g., a patient interface, conduit headgear, or the like).

The first tag 9200A and the second tag 9200B may each be read-only, read/write, write once, read many, or any combination of the like. The first tag 9200A and the second tag 9200B may each be configured to include identifying data of conduit headgear 4180, the patient interface 3000, and/or the patient. In some aspects, at least one of the first tag 9200A and the second tag 9200B may include information related to a date of usage or a time stamp. The first tag 9200A and the second tag 9200B may each additionally or alternatively include information related to the type of the conduit headgear 4180 being used, the type of the patient interface 3000 being used, characteristics of the conduit headgear 4180 or the patient interface 3000 (e.g., one or more of cushion material, cushion size, conduit size, patient interface size, length of use of the patient interface or conduit headgear, date of manufacture of the patient interface or conduit headgear, a set of respiratory therapy conditions the patient interface or conduit headgear is suitable for use with, etc.), a serial identification number of the patient interface 3000 or the conduit headgear 4180, and/or other aspects of the respiratory therapy system 8000. In some aspects, the first tag 9200A and/or the second tag 9200B may be configured to include patient information, e.g., the type of therapy or therapy settings the patient is intended to receive, or other information.

In one example, in reference to FIG. 6, the first tag 9200A may be associated with the conduit headgear 4180 and may be configured to store identifying data of the conduit headgear 4180. The second tag 9200B may be associated with the patient interface 3000′ or a cushion of the patient interface 3000′ and may be configured to store identifying data of patient interface 3000′ or a cushion of the patient interface 3000′. Upon connecting the air circuit 4170 to the conduit headgear 4180, the first antenna 9100A may detect the first tag 9200A and receive the identifying data of the conduit headgear 4180. The third antenna 9100C may detect second tag 9200B and receive the identifying data of the patient interface 3000′ or a cushion of the patient interface 3000′, which may then be transmitted along the electrical connection 9105 to second antenna 9100B. The first antenna 9100A may then read the data from the second antenna 9100B.

As discussed above, although two tags 9200 are depicted in the accompanying figures, only one tag 9200 may be incorporated in RFID system 9000 in some aspects. In other aspects, more than two tags, e.g., three, four, five, or more tags, may be incorporated in the RFID system 9000. If multiple tags are incorporated in the RFID system 9000, an anti-collision mechanism may be enabled to allow the reader to accurately read one or more tags simultaneously.

In some embodiments, the first tag 9200A may include an adhesive, e.g., to assist in applying the first tag 9200A to conduit headgear 4180 and/or maintaining the first tag 9200A in place on the conduit headgear 4180 after application. The first tag 9200A may alternatively be overmolded within or integrally formed on a portion of the conduit headgear 4180. Alternatively, the first tag 9200A may be comprised of conductive silicone and/or conductive thread(s) directly on to a portion of the conduit headgear 4180. In some aspects, the first tag 9200A may be incorporated as part of a sleeve fitted onto the conduit headgear 4180. The first tag 9200A may be coupled to the conduit headgear 4180 in any suitable method commonly used in the art.

Similarly, the second tag 9200B may include an adhesive, e.g., to assist in applying the second tag 9200B to the patient interface 3000 or a cushion thereof and/or maintaining the second tag 9200B in place on the patient interface 3000 after application. The second tag 9200B may alternatively be overmolded within or integrally formed on a portion of the patient interface 3000. Alternatively, the second tag 9200B may be comprised of conductive silicone and/or conductive thread(s) directly on to a portion of patient interface 3000. Additionally or alternatively, ink (e.g., silver ink) may be printed onto a substrate, such as a fabric, to form the second tag 9200B, or the tag 9200B may include an overmoulded inlay label or other conductive textile. The second tag 9200B may be coupled to patient interface in any suitable method used in the art.

The first tag 9200A and the second tag 9200B may each be RFID tags, and, in some instances, may each be near-field communication (NFC) tags. Alternatively, the tags 9200A and 9200B may be ultra high frequency (“UHF”), Bluetooth, or ultra wideband (UWB) tags. The first tag 9200A and the second tag 9200B may each be configured to generate an electromagnetic field with a frequency of about 13.56 megahertz (MHz). For example, the first antenna 9100A may be configured to read data transmitted from the first tag 9200A at 13.56 MHz. Similarly, the third antenna 9100C may be configured to read data transmitted from the second tag 9200B at about 13.56 MHz. The first antenna 9100A may also be configured to read data transmitted from the second antenna 9100B at about 13.56 MHz.

In other aspects, the RFID system 9000 may alternatively be configured to generate an electromagnetic field with a frequency between about 30 kilohertz (kHz) and about 12 gigahertz (GHz) (e.g., from approximately 10 MHz to approximately 12 GHz). In some examples, the RFID system 9000 may be configured to operate in a low frequency range, or between about 30 kHz to 300 kHz. Accordingly, the RFID system 9000 may be configured to have a read range of up to approximately 10 centimetres (approximately 3.94 inches), although the exact distance may vary depending, e.g., on the angles of the various components in the system relative to one another. Alternatively, the RFID system 9000 may be configured to operate in a high frequency range, or between about 3 MHz and 30 MHz. In such a configuration, the RFID system 9000 may be configured to have a read range between approximately 10 centimetres up to approximately one metre (approximately 3.94 inches to approximately 39.37 inches). Further still, the RFID system 9000 may be configured to operate in an ultra-high frequency range, or between about 300 MHz and 3 GHz. In such a configuration, the RFID system 9000 may be configured to have a read range between approximately one metre up to approximately 12 metres.

In some examples, a shorter read range for the RFID system 9000 may be desirable. For example, if the patient is in the vicinity of multiple device or objects containing RFID components, the RFID system 9000 may inadvertently read the tag or antenna on the peripheral devices. Accordingly, if the RFID system 9000 is configured to operate on a low frequency range, the possibility for the RFID system 9000 to read a peripheral device inadvertently is decreased. Extended antenna configurations described herein may facilitate the use of shorter reading ranges for the RFID system 9000 even when an RFID tag is spaced apart from an antenna and transceiver.

First, second, and third antennas 9100A, 9100B, and 9100C may be set to the same inductance for the first and second tags 9200A and 9200B for different types of, e.g., the patient interface 3000, the conduit headgear 4180, or other accessory device, so that the antennas 9100 may be compatible with a variety of patient interface components.

The RFID system 9000 may include one first tag 9200A fixedly or removably coupled to the conduit headgear 4180 and/or one second tag 9200B fixedly or removably coupled to the patient interface 3000 or to an accessory device (not shown). Alternatively, the RFID system 9000 may include two or more first tags 9200A fixedly or removably coupled to the conduit headgear 4180 and/or two or more second tags 9200B fixedly coupled to the patient interface 3000 or to an accessory device (not shown). The first tag 9200A and/or the second tag 9200B described herein may be off-the-shelf components or may be customized according to a size and/or a shape of the patient interface 3000 or the conduit headgear 4180 and/or according to a desired read range for the first tag 9200A and the second tag 9200B.

The first antenna 9100A may be configured to receive data from the second antenna 9100B and the first tag 9200A (as noted herein, both 9100B and 9200A may be embodied within the same RFID tag). The first antenna 9100A is also configured to transmit the data received from the second antenna 9100B and the first tag 9200A to transceiver, or the reader, 9300. The transceiver 9300 may be operably connected to the first antenna 9100A, e.g., physically (via a wire) and may be located on the air circuit 4170. In one configuration, a transceiver 9300 may be external to the respiratory therapy system 8000. For example, the transceiver 9300 may be a scanner, a smartphone, a tablet, or any other device configured to receive transmitted RFID signals from an RFID tag or antenna. Accordingly, the first antenna 9100A is configured to communicate data received from one or more tags 9200 to the transceiver 9300. The transceiver 9300 may also be configured to save or store the transmitted data from the first antenna 9100A.

The transceiver 9300 may relay information from the first antenna 9100A to a controller configured to control the RPT device 4000 (e.g., the central controller 4230 and/or the therapy control module 4330). The controller may be separate from the RPT device 4000 or may be incorporated as part of the RPT device 4000. The controller may function as described above. In some configurations, the transceiver 9300 may be configured to transmit data to the RPT device 4000 physically (e.g., via a wire) or wirelessly. The RPT device 4000 may also be configured to save or store the transmitted data from the transceiver 9300, interpret the transmitted data, and/or transmit an alert or signal to the user or care provider, as described above. In some embodiments, RPT device 4000 may also be configured to automatically change one or more characteristics of the respiratory pressure therapy, for example, based on the raw data and/or interpreted data received from the transceiver 9300. Additionally or alternatively, the RPT device 4000 may be configured to suggest one or more therapy settings, for example, to promote patient care and/or patient comfort based on the interpreted data from the transceiver 9300, as will be discussed further below.

Although not shown in FIG. 6, the transceiver 9300 may be a combined with the first antenna 9100A. For example, the transceiver 9300 and the first antenna 9100A may form a single combined component. The combined component may have any or all of the characteristics of the first antenna 9100A and the transceiver 9300 described above. For example, the combined first antenna 9100A and transceiver 9300 may be configured to send or receive data to a controller e.g., of the RPT device 4000, such as the central controller 4230.

FIG. 6 illustrates a perspective view of an exemplary patient interface 3000′ and the conduit headgear 4180. The patient interface 3000′ may be used with the system 8000, described above. The conduit headgear 4180 is a conduit or a tube constructed and arranged to allow, in use, a flow of air to travel between two components of a respiratory therapy system such as, for example, air circuit 4170 and patient interface 3000′.

A proximal portion 4170A of the air circuit 4170 may be coupled to a second portion 4180B of the conduit headgear 4180. The air circuit 4170 may be coupled to the conduit headgear 4180 via, for example, a joint 4190. The joint 4190 may permit rotation and movement of the air circuit 4170 while still maintaining a connection between the air circuit 4170 and the conduit headgear 4180. In alternative embodiments, the air circuit 4170 is coupled directly to the conduit headgear 4180.

The conduit headgear 4180 may bifurcate or branch into a first portion 4180A and a second portion 4180B. A distal portion of the first portion 4180A of the conduit headgear 4180 may include a flexible or stretchable portion, such as a first concertina portion 4200A, and the second portion 4180B of conduit headgear 4180 may include a flexible or stretchable portion, such as a second concertina portion 4200B. The first concertina portion 4200A and the second concertina portion 4200B may be substantially the same for the purposes of this description. For example, the first concertina portion 4200A may extend a first length along the first portion 4180A of the conduit headgear 4180, and the second concertina portion 4200B may extend the same length along second portion 4180B of the conduit headgear 4180. Each of the first concertina portion 4200A and the second concertina portion 4200B may be corrugated. Accordingly, the first concertina portion 4200A and/or the second concertina portion 4200B may extend and/or compress, for example, to adjust the conduit headgear 4180 to a patient's face. The first concertina portion 4200A and/or the second concertina portion 4200B may extend along an entire length of the conduit headgear 4180 or may extend along less than an entire length of the conduit headgear 4180, e.g., along a majority of a length of the conduit headgear 4180, or less than a majority of a length of conduit headgear 4180. In the illustrated example, each of the first portion 4180A and the second portion 4180B includes a concertina portion, i.e., first concertina portion 4200A and the second concertina portion 4200B. In an alternative example, both the first portion 4180A and the second portion 4180B may not include a concertina portion on both sides.

It may be challenging to incorporate a wire extending along the conduit headgear 4180 as a result of the flexibility and stretchability of the conduit headgear 4180. In particular, it may be difficult for the electrical connection 9105 to be bonded on or within the conduit headgear 4180 as a result of the first concertina portion 4200A and/or the second concertina portion 4200B. The electrical connection 9105 may need to maintain the correct antenna impedance while being stretchable. One or more of these challenges may be addressed by aspects of the technology described herein.

Also, it should be appreciated that aspects of the technology may be applicable to a single limb conduit, e.g., a single conduit arranged in a tube-up type configuration in which the conduit extends to a position at the top of the patient's head (e.g., Adam's circuit) and/or a single conduit arranged in a tube-down configuration in which the conduit hangs down from the front of the patient's face. For example, the single conduit may include a portion configured to extend from a distal end to a proximal end (e.g., the distal end configured to couple with an air circuit and the proximal end configured to couple with a patient interface), a stretchable concertina portion configured to extend along a length of the portion, and an electrical connection (e.g., a stretchable conductor) configured to extend along at least a portion of the stretchable concertina portion. The single conduit is sometimes referred to as a “short tube” and extends from the patient interface to the main air delivery tube from the flow generator. Such single conduit short tube may be able to expand or stretch in a longitudinal sense, e.g., 5-100% stretch, from a neutral position to a stretched position upon application of a load (e.g., gravity and/or tube drag), and at least partially return to the neutral position due to elastic resiliency upon lessening or removal of the load. The single conduit may also be able to be compressed in a longitudinal sense.

As previously discussed, the first antenna 9100A may be disposed on or within a proximal portion 4170A of air circuit 4170. The second antenna 9100B may be disposed on or within a distal portion of the conduit headgear 4180 configured to couple to the air circuit 4170. The first tag 9200A may be adjacent to or near the first antenna 9100A, for example, within the second portion 4180B of the conduit headgear 4180. In an example, the second antenna 9100B is provided as a part of the first tag 9200A (e.g., as the antenna part of an RFID tag). In an example, the second antenna 9100B is provided separately from the first tag 9200A (e.g., the antenna part of an RFID tag may be separate from second antenna 9100B). A distal end of the electrical connection 9105 is coupled to the second antenna 9100B. The electrical connection 9105 may extend from the second antenna 9100B, on or within a distal portion of the first portion 4180A of the conduit headgear 4180, to a proximal portion of the first portion 4180A. The electrical connection 9105 may be external of the conduit headgear 4180 or may extend within the material comprising the conduit headgear 4180 or within a coating applied to the conduit headgear 4180. In one example, the electrical connection 9105 may be integrated within the material comprising the conduit headgear 4180. As discussed above, in some aspects, the electrical connection 9105 may not be positioned within an air flow passage of the conduit headgear 4180.

A proximal end of the electrical connection(s) 9105 is coupled to the third antenna 9100C. The third antenna 9100C is disposed on or within a proximal portion of the air circuit 4170, for example, within a proximal portion of the first portion 4180A of the conduit headgear 4180. A proximal end of the first portion 4180A of conduit headgear 4180 may be coupled directly or indirectly to the patient interface 3000′. The second tag 9200B may be associated with the patient interface 3000′, a cushion, or one or more other accessories. Although the electrical connection 9105 and the third antenna 9100C are depicted in reference to the first portion 4180A, the second portion 4180B may also incorporate similar structures and conduit headgear may be substantially symmetrical. In some aspects, however, one of the first portion 4180A or the second portion 4180B may incorporate the third antenna 9100C and the electrical connection 9105, while the other of the first portion 4180A or the second portion 4180B may not. As noted above, the first portion 4180A and/or the second portion 4180B may include a concertina portion, and the third antenna 9100C and the electrical connection 9105 may be provided to the first portion and/or the second portion.

In one aspect, to accommodate the stretchability of flexibility of the first concertina portion 4200A and the second concertina portion 4200B, the electrical connection 9105 may extend along a serpentine path. As shown in FIG. 6, the electrical connection 9105 forms a plurality of the curves 9106. The plurality of curves 9106 may extend along a portion of the electrical connection 9105 or along an entirety of the electrical connection 9105. For example, the plurality of curves 9106 may extend along at least the first concertina portion 4200A.

Each curve of the plurality of curves 9106 may be dimensionally the same or similar. Alternatively, one or more curves of the plurality of curves 9106 may dimensionally differ. For example, one or more curves of the plurality of curves 9106 may be larger or small to accommodate the curvature of the conduit headgear 4180 or changes in corrugation of the concertina portion. By arranging the electrical connection 9105 with the plurality of curves 9106 or, for example, in a zig-zag or serpentine path, stretching or bending of the conduit headgear 4180 may result in relatively little increase in tensile forces on the electrical connection 9105. This configuration may also reduce the chances of the electrical connection 9105 failing due to repeated stretching and/or bending cycles.

In another aspect, the electrical connection 9105 may itself have a coiled or serpentine configuration to allow for stretchability. An electrical connection 9105 with a coiled or serpentine configuration may extend along a more direct path from the second antenna 9100B to the third antenna 9100C, or may follow a serpentine or zig-zag path. The electrical connection 9105 itself may or may not be formed of a stretchable material.

In some aspects, the electrical connection 9105 may be formed of a stretchable conductive material and may be incorporated on or within a sleeve 9107, demonstrated by the stippling portion in FIG. 6. The sleeve 9107 may overlay the conduit headgear 4180 and may be removable relative to the conduit headgear 4180, or the sleeve 9107 may be fixedly coupled to an outer surface of the conduit headgear 4180 (e.g., to the first portion 4180A of the conduit headgear 4180 (as illustrated), to the second portion 4180B, or to both the first portion 4180A and the second portion 4180B). The sleeve 9107 may surround (e.g., wrap around) a portion of the conduit headgear 4180 or surround an entire circumference of the conduit headgear 4180. Additionally or alternatively, the sleeve 9107 may extend along an entire length or less than an entire length of the first portion 4180A and/or the second portion 4180B of conduit headgear 4180. In some aspects, the sleeve 9107 may cover a majority of or substantially all of an outer surface of the conduit headgear 4180 including, e.g., around an opening in the conduit headgear 4180 configured to couple to air circuit 4170.

The sleeve 9107 may be comprised of one or more materials. For example, the sleeve 9107 may be comprised of one or more of a flexible conductive silicone, conductive fibers or threads, a stretchable conductive textile or other suitable materials or combinations of materials. The material may include conductive traces, e.g., copper traces, or conductive fibers or threads that may be printed, thermally bonded, sewn, or otherwise incorporated onto or into the material. In some aspects, a metal gel soft circuit may be used to form the electrical connection(s) 9105. The metal gel soft circuit may comprise a membrane that may be bonded to a textile version of the sleeve 9107. Additionally, the metal gel soft circuit may include one or more stabilizing additives to allow the material to withstand high-pressure lamination and thermo-welding processes without displacement. The metal gel soft circuit may be configured to withstand bending, stretching, or torsion.

The sleeve 9107 may be comprised of flexible materials and/or stretchable materials and may include a corrugated portion or be configured to stretch or otherwise accommodate a corrugated portion, such as the first concertina portion 4200A and/or the second concertina portion 4200B of the conduit headgear 4180. For example, the sleeve 9107 may be configured to be extended or stretched and compressed to accommodate the length of the first portion 4180A or the second portion 4180B of conduit headgear 4180 as it changes.

Additionally or alternatively, the sleeve 9107 may also include the second antenna 9100B and the third antenna 9100C. For example, the second antenna 9100B may be disposed on or within a first end of the sleeve 9107 (e.g., near or adjacent to the attachment location of the air circuit 4170), and/or third antenna 9100C may be disposed on or within a second end of the sleeve 9107 (e.g., near or adjacent to patient interface 3000′). If the sleeve 9107 is configured to cover both the first portion 4180A and the second portion 4180B of the conduit headgear 4180, then one or more additional antennas may be disposed on or within the sleeve 9107, e.g., if approximately symmetrical antenna extensions are provided along both the first portion 4180A and the second portion 4180B of the conduit headgear 4180.

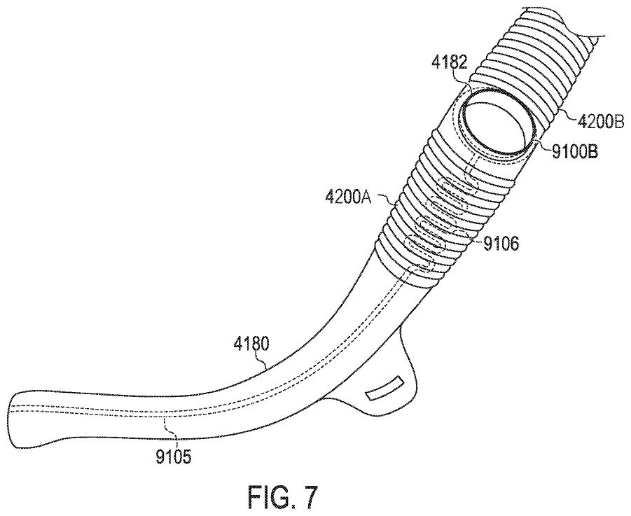

FIG. 7 illustrates a top perspective view of an exemplary portion of the conduit headgear 4180. In this configuration, the second antenna 9100B encircles or surrounds an opening 4182 where the air circuit 4170 is configured to couple to the conduit headgear 4180. The second antenna 9100B may be, for example, incorporated into a material from which the conduit headgear 4180 is made surrounding the opening 4182, may be printed, thermally bonded, adhered, or coated around the opening 4182, or may be incorporated into a sleeve, e.g., a textile sleeve, that fits over the conduit headgear 4182 and surrounds the opening 4182. The opening 4182 is configured to be coupled (e.g., directly or indirectly) to the air circuit 4170. Although not shown, in some configurations, the first antenna 9100A encircles or surrounds a corresponding opening on the air circuit 4170.

To accommodate the corrugations of the first concertina portion 4200A, the electrical connection(s) 9105 may include a distal portion 9105A having a plurality of curves 9106. The plurality of curves 9106 may extend along an entire length of the first concertina portion 4200A or along a portion of the first concertina portion 4200A. Accordingly, the plurality of curves 9106 may include one curve, two curves, three curves, etc. The plurality of curves 9106 may enable the electrical connection(s) 9105 to extend and/or contract, for example, as the first concertina portion 4200A is expanded and/or contracted. The electrical connection(s) 9105, including the plurality of curves 9106, may be formed from a stretchable conductive silicone. In some embodiments, the stretchable conductive silicone may be threads or fibers printed, thermally bonded, adhered, overmolded, or coated to the material from which the conduit headgear 4180 is formed.

Additionally or alternatively, the electrical connection(s) 9105 may be formed from a stretchable conductive textile with printed conductive, e.g., copper traces. The stretchable conductive textile with printed conductive, e.g., copper, traces may be thermally bonded to the conduit headgear 4180, which may be formed, e.g., of silicone. Additionally or alternatively, a metal gel soft circuit may be used to form the electrical connection(s) 9105. The metal gel soft circuit may comprise a membrane that may be bonded to the conduit headgear 4180, which may be formed of a silicone part. A metal gel soft circuit may alternatively comprise a membrane that may be bonded onto a textile. Additionally, the metal gel soft circuit may include one or more stabilizing additives to allow the material to withstand high-pressure lamination and thermo-welding processes without displacement. The metal gel soft circuit may be configured to withstand bending, stretching, or torsion. Any one or combination of these materials may be utilized to form and incorporate the electrical connection(s) 9105 and/or one or more antennas to the conduit headgear 4180.

Although not shown in the accompanying figures, conductive stretchable materials described herein may also be used to interconnect one or more sensors, electrodes, accelerometers, high elongation strain gauges, microprocessors, and/or other circuitry elements that may be present on the patient interface 3000, the conduit headgear 4180, the air circuit 4170, or other accessories.

During use of the patient interfaces 3000, 3000′, or another suitable patient interface (collectively patient interface 3000), the air circuit 4170 may be fluidly coupled to the RPT device 4000 and to the conduit headgear 4180. As described above, the RPT device 4000 may be configured to supply a flow of gas, for example, air, which may be supplemented with oxygen, through the air circuit 4170 and the conduit headgear 4180 to the patient interface 3000. The RPT device 4000 may also be configured to receive a signal from a proximal end of the air circuit 4170 once the air circuit 4170 is coupled to the conduit headgear 4180. Additionally or alternatively, the RPT device 4000 may be configured to receive a signal from a proximal end of the conduit headgear 4180 once the conduit headgear 4180 is coupled to the patient interface 3000. The signal may include information regarding patient interface 3000 or an accessory. For example, the first antenna 9100A associated with the air circuit 4170 may detect the first tag 9200A in the conduit headgear 4180 and may read information about the conduit headgear 4180 associated with the first tag 9200B when the air circuit 4170 is coupled to the conduit headgear 4180. Similarly, the third antenna 9100C in the conduit headgear 4180 may detect the second tag 9200B in the patient interface 3000 and may relay information about the patient interface 3000 associated with the second tag 9200B to the second antenna 9100B, which may be read by the first antenna 9100A. Via the transceiver 9300, for example, this information may be transmitted to a controller of the RPT device 4000 (e.g., the central controller 4230) or a controller associated with the system 8000.

In some aspects, the received information (e.g., from the tag 9200B) may be one or more of, e.g., the type of patient interface 3000 being used, characteristics of the patient interface 3000 (e.g., one or more of cushion material, cushion size, conduit size, patient interface size, length of use of the patient interface, date of manufacture of the patient interface, a set of respiratory therapy conditions the patient interface is suitable for use with, etc.), date of usage or time stamp, batch identification number of the patient interface 3000, or a serial identification number of patient interface 3000. In some aspects, the received information may be one or more of patient information, e.g., the type of therapy or therapy settings the patient is intended to receive, or other information. In some aspects, the received information may be whether the air circuit 4170, the conduit headgear 4180, and/or an accessory device (not shown) is/are connected or disconnected from the patient interface 3000. Similar information may be received, e.g., about the conduit headgear 4180, for example, from the tag 9200A.

The RPT device 4000 (namely a controller of the RPT device 4000, such as the central controller 4230, which may be incorporated as part of or separate from the RPT device 4000), may be configured to perform an action upon receiving information from transceiver 9300 about patient interface 3000. For example, the RPT device 4000 may automatically control operation of the therapy provided to the patient based on the information or signal received (e.g., the therapy device controller 4240 and or the therapy control module 4330). For example, the breathing experience may be improved by determining whether the settings of the RPT device 4000 (e.g., gas flow, humidity levels, etc.) are correctly aligned to the patient interface being worn by the patient. In some configurations, the RPT device 4000 may enable the design of patient interface-specific venting and flow curves to promote more comfortable and/or efficient delivery of therapy. In some aspects of the technology, an indication to the patient may be generated based on the information received by the RPT device 4000. For example, an indication may be generated that the incorrect type or size of patient interface or conduit headgear is being used, that a cushion of the patient interface or the entire patient interface or conduit headgear should be replaced, that the RPT device 4000 is configured with one or more incorrect settings that should be changed by the patient, or other suitable indications. The indication may be generated, e.g., on one or more of a display of the RPT device 4000 (e.g., the display 4294) or other component of the system 8000, to an external device, such as the remote external device 4286 or the local external device 4288 (e.g., a patient's tablet, smartphone, or computer, or to a healthcare provider).

In further aspects of the technology, information received from the patient interface 3000 may be received by the RPT device 4000 in addition to information received from other sensors or systems associated with the system 8000. For example, sensors or systems configured to detect air flow, pressure, air leaks, humidity, or other characteristics of the system 8000 may transmit information to the RPT device 4000. The RPT device 4000 may analyze or interpret information regarding the patient interface 3000 or conduit headgear received from the RFID system 9000 in combination with one or more other sensors. For example, information may be received from the RFID system 9000 regarding the type or size of patient interface being worn or how long the patient interface has been used for.

The system 8000 may also receive information from other sensors or patient input regarding patient interface discomfort or the occurrence of leaks. The system 8000 may analyze this information together and may take an action based on the aggregate information. For example, if patient interface discomfort is indicated by a patient or leaks are detected with the patient interface, based on the information received from the RFID system 9000, the RPT device 4000 may generate an indication to a patient that a difference size or type of patient interface or conduit headgear should be used, a different size or type of cushion should be used, or a new patient interface, conduit headgear, or cushion should be used. This indication may include, e.g., a suggestion regarding a patient interface, conduit headgear, or cushion type or size to be used or guidance on how to select a better-fitting cushion, conduit headgear, or patient interface type or size. Indications may be generated as described above, e.g., on one or more of a display of the RPT device 4000 (e.g., the display 4294) or other component of the system 8000, to an external device, such as the remote external device 4286 or the local external device 4288 (e.g., a patient's tablet, smartphone, or computer, or to a healthcare provider). In other aspects, one or more settings of the RPT device 4000 may be changed based on the aggregate information received from the RFID system 9000 and other information from the system 8000.

In some aspects, the system 8000 may indicate to a patient when a patient interface, conduit headgear, or cushion has been worn long enough or is an old enough production batch identification number or a serial identification number that performance may be impacted and a new patient interface, conduit headgear, or cushion should be used. In some aspects, the age of the patient interface or conduit headgear or how many times the patient interface or conduit headgear has been used may lead to generation of an indicator that a new patient interface, new conduit headgear, or new cushion should be used. The system 8000 may indicate a patient to replace the patient interface, conduit headgear, or cushion. This indication may also depend, at least in part, on the recommended usage period of a particular patient interface, conduit headgear, or cushion type, the type of therapy being administered to the patient, or one or more other factors. Indications may be generated as described above, e.g., on one or more of a display of RPT device 4000 or other component of system 8000, to an external device such as the remote external device 4286 or the local external device 4288, (e.g., a patient's tablet, smartphone, or computer, or to a healthcare provider), or to a cloud server, such as remote external communication network 4282 and/or local external communication network 4284 and/or remote external communication network 4282 (e.g., for remote monitoring, to improve patient experience, to trigger an order (such as for a new patient interface or accessory, etc.)).

First antenna 9100A and transceiver 9300 may read a signal from first tag 9200A and/or second tag 9200B via extended second antenna 9100B at least once during a therapy session. For example, first tag 9200A and/or second tag 9200B may be read when therapy starts, e.g., when a start button is pressed or when auto-start is initiated. In some aspects, if no accessory or conduit headgear 4180 is detected as being attached to an air circuit 4170, then RFID system 9000 may continue to read, e.g., continuously or at regular intervals until the accessory or conduit headgear 4180 is detected as being attached to air circuit 4170. In some configurations of the technology, a signal from first tag 9200A may be read periodically, e.g., at every few seconds, minutes, or hours. In some examples of the technology, first antenna 9100A and transceiver 9300 may read the signal from first tag 9200A and/or second tag 9200B according to a regular or irregular frequency. For example, the signal may be read more or less frequently at the beginning of a session or after a pre-determined amount of time. In some aspects, the frequency may increase if an unexpected reading occurs, or in the event of a failed reading. In some aspects, the type of therapy being administered or type of patient interface being used may at least in part determine the frequency with which the first tag 9200A and/or the second tag 9200B is read.

FIG. 5B is a schematic view of system 8000 according to certain examples. Air circuit 4170 includes a transceiver 9300′ and an RFID reader 9100A′. Conduit headgear 4180 includes tag 9200A′ and RFID reader 9100C′. Patient interface includes tag 9200B′. In certain examples, the various discussions of antenna 9100A and/or transceiver 9300 herein can be applied to RFID reader 9100A′ and vice versa. In certain examples, the various discussions of antenna 9100B and/or tag 9200A herein can be applied in a similar manner to tag 9200A′ and vice versa. In certain examples, the various discussions of tag 9200B can be applied in a similar manner to tag 9200B′ and vice versa.

The transceiver 9300′ may be configured to handle communication (via wire or wirelessly) to RPT device 4000 or other devices.

RFID reader 9100A′ is configured to wirelessly communicate and/or power tag 9200A′ that is provided in conduit headgear 4180. RFID reader 9100A′ includes at least one antenna, which may correspond to antenna 9100A in certain examples, and an integrated circuit that includes a transceiver (e.g., the transceiver 9300 from FIG. 5A in an example). Tag 9200A′ may be an RFID tag that includes at least one antenna, which may correspond to antenna 9100B in certain examples, and an integrated circuit, which may correspond to tag 9200A in certain examples. Tag 9200A′ may also include non-transitory memory for storing data thereon.

Tag 9200A′ is connected via a wire (as discussed elsewhere herein) to RFID reader 9100C′. The wire allows energy that is received by tag 9200A′ to be carried to RFID reader 9100C′ to thereby provide wireless communication and/or power to tag 9200B′ that is provided in patient interface 3000. The RFID reader 9100C′ includes at least one antenna, which may correspond to antenna 9100C in certain examples, and an integrated circuit that includes a transceiver, which may be the same or similar to that in RFID reader 9100A′. Tag 9200B′ may be an RFID tag that includes at least one antenna and an integrated circuit, which may correspond to tag 9200B in certain examples. Tag 9200B′ may include non-transitory memory for storing data thereon.

The reader/tag combinations herein act as, and are in certain examples, air core transformers. For example, RFID reader 9100A′ is the primary winding of a first transformer, with 9200A′ being the secondary winding of the first transformer. Similarly, 9100C′ may be the primary winding of a second transformer, with 9200B′ being the secondary winding of the second transformer. Accordingly, for example, RFID reader 9100A′ is used to generate an electromagnetic field that powers tag 9200A′. The power output from the RFID reader 9100A′ and/or 9100C′ can be modulated in order to transmit commands. The tags can answer these commands by modulating their respective antenna impedance. This modulation is then detected by a corresponding RFID reader and interpreted as data.

In some aspects, wireless power that is supplied from air circuit (e.g., via RFID reader 9100A′) may be relatively increased in comparison to the wireless power needed to just read or otherwise activate a tag. This is because the power supplied from RFID reader 9100A′ may also be used (in addition to reading tag 9200A′) to power RFID reader 9100C′ to read tag 9200B′. In an example, the activation of the RF field from RFID reader 9100A′ may be maintained for a relatively brief amount of time, such as less than one second, or between 1 and 10 seconds. The time period that wireless energy is supplied to the conduit headgear 4180 may be set/determined in order to allow for reading data from the tags as discussed herein.

In an example, a reader included in the air circuit may be the same type as the reader included in the conduit headgear. In other examples, the readers may be different types. For example, the reader in air circuit may be designed to have a relatively higher energy transfer value to the tag of the conduit headgear than that of the reader of the conduit headgear to the tag of the patient interface.

In an example, when multiple tags are included in system 8000, they may be the same type of tag. In another example, different tags of system 8000 may be of different types. As an example, a tag included in a patient interface may be an active tag, while the tag included in the conduit headgear 4180 may be passive. In an example, different types of passive tags may be used.

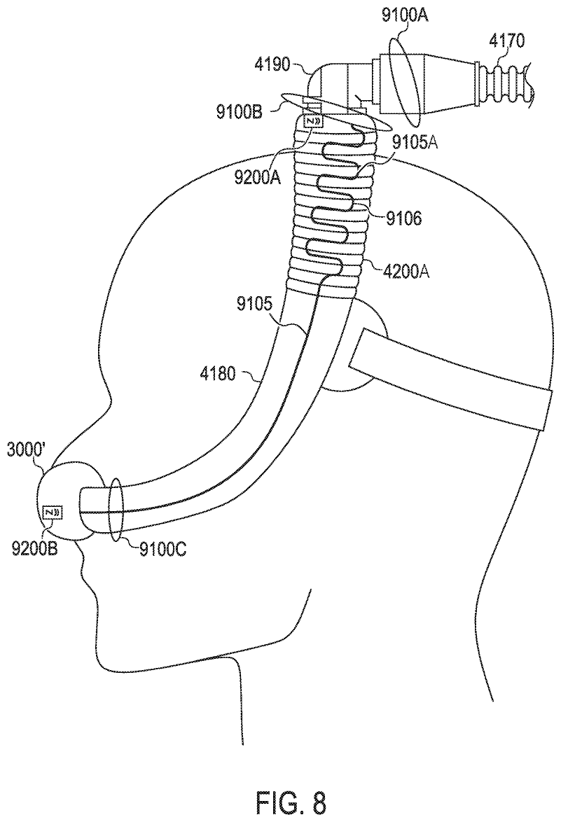

FIG. 8 illustrates the patient interface 3000′, the conduit headgear 4180, and the air circuit 4170 in fluid communication with one another while being worn by a patient. For example, the conduit headgear 4180 is configured such that the air circuit 4170 is coupled to the conduit headgear 4180 at or near the top of the patient's head, for example, via the joint 4190 (discussed above with respect to FIG. 6). The air circuit 4170 may be rotatable relative to conduit the headgear 4180 via the joint 4190. For example, the joint 4190 may be a swivel joint, thus permitting the air circuit 4170 to rotate relative to the conduit headgear 4180. Additionally, the conduit headgear 4180 and the air circuit 4170 may be angled relative to one another. For example, via the joint 4190, the air circuit 4170 may be approximately 90 degrees relative to the conduit headgear 4180. Accordingly, in some aspects, the first antenna 9100A, which may be fixed in or on proximal portion of the air circuit 4170, may be angled relative to the second antenna 9100B fixed in or on the conduit headgear 4180.

As previously discussed, the electrical connection(s) 9105 physically and electrically couple second the antenna 9100B to the third antenna 9100C. The electrical connection(s) 9105 may include a plurality of curves 9106 along the first concertina portion 4200A or the second concertina portion 4200B (FIGS. 6 and 7), thus permitting the electrical connection(s) 9105 to extend and/or contract, for example, as the first concertina portion 4200A is extended and/or contracted. In such a way, signals from the third antenna 9100C may be transmitted to second antenna 9100B via the electrical connection(s) 9105.

As discussed above, the electrical connection(s) 9105 may include a plurality of curves 9106. The plurality of curves 9106 may extend along a portion of the electrical connection 9105, as shown. By arranging the electrical connection 9105 with the plurality of curves 9106 or, for example, in such a zig-zag/serpentine path, stretching or bending of the conduit headgear 4180 may result in relatively little increase in tensile forces on the electrical connection 9105. This may allow use of an electrical connection 9105 that has relatively little stretch or an electrical connection 9105 that is stretchable. This configuration may also reduce the chances of the electrical connection 9105 failing due to repeated stretching and/or bending cycles.

Each embodiment discussed herein may enable the user to detect the identification of the patient interface or accessory being used. In such a way, information regarding the mask, the accessory, the patient, or the therapy prescribed may be transmitted via RFID. Each embodiment may help to increase patient use and/or patient comfort, among other things.

It will be apparent to those skilled in the art that various modifications and variations can be made to the disclosed device without departing from the scope of the disclosure. Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

### 5.6 Reference Signs List

| Reference No. | Reference Label |
|---|---|
| 1000 | patient |
| 1100 | bed partner |
| 3000 | patient interface |
| 3000′ | patient interface |
| 3000-1 | tube up nose and mouth patient interface |
| 3000-2 | nose and mouth patient interface |
| 3000-3 | patient interface |
| 3000-4 | nose only patient interface |
| 3012A | first nasal cushion |
| 3012B | second nasal cushion |
| 3014A | first hole |
| 3014B | second hole |
| 3016 | lumen |
| 3018 | tubular portion |
| 3018A | first portion |
| 3018B | second portion |
| 3018C | third portion |
| 3020A | first end |
| 3020B | second end |
| 3022 | features |
| 3050-1 | cushion |
| 3050-2 | cushion |
| 3100 | seal-forming structure |
| 3200 | plenum chamber |
| 3200-1 | plenum chamber |
| 3200-2 | plenum chamber |
| 3254 | plenum chamber inlet port |
| 3254-1 | plenum chamber inlet ports |
| 3254-2 | plenum chamber inlet ports |
| 3266-1 | groove |
| 3266-2 | groove |
| 3300 | structure |
| 3302 | headgear |
| 3302-1 | headgear |
| 3302-2 | headgear |
| 3304-1 | inferior strap |
| 3305-1 | superior strap |
| 3306-1 | magnetic member |
| 3307-1 | rear strap |
| 3307-2 | rear strap |
| 3308-2 | intermediate section |
| 3309-2 | slit |
| 3320 | tab |
| 3328 | concertina structure |
| 3332 | inlet |
| 3340 | rigidiser arm |
| 3342 | first end |
| 3344 | second end |
| 3350 | tube |
| 3351 | conduit sleeve |
| 3352 | superior opening |
| 3354 | inferior extension |
| 3356 | connection member |
| 3362 | extendable concertina structure |
| 3363 | non-extendable tube sections |
| 3370-1 | magnets |
| 3380 | four-point arm sleeve |
| 3380-1 | sleeve |
| 3384 | inferior extension |
| 3386 | connection member |
| 3388-1 | inferior opening |
| 3394 | tabs |
| 3394-1 | tabs |
| 3400 | vent |
| 3402 | vent opening |
| 3402-1 | vent opening |
| 3402-2 | vent opening |
| 3404 | vent housing |
| 3408 | anterior surface |
| 3412 | posterior surface |
| 3416 | groove |
| 3448 | diffuser |
| 3450 | vent |
| 3456 | anterior surface |
| 3464 | gap |
| 3500 | conduit connection structure |
| 3504 | connection structure |
| 3600 | connection port |
| 3610 | elbow |
| 3700 | forehead support |
| 3800 | patient interface |
| 4000 | RPT device |
| 4010 | external housing |
| 4020 | pneumatic block |
| 4110 | air filter |
| 4112 | inlet air filter |
| 4114 | outlet air filter |
| 4120 | muffler |
| 4122 | inlet muffler |
| 4124 | outlet muffler |
| 4140 | pressure generator |
| 4142 | blower |
| 4144 | motor |
| 4160 | anti-spill back valve |
| 4170 | air circuit |
| 4170A | proximal portion |
| 4180 | conduit headgear |
| 4180A | first portion |
| 4180B | second portion |
| 4182 | opening |
| 4190 | joint |
| 4192 | supplemental gas |
| 4200A | first concertina portion |
| 4200B | second concertina portion |
| 4202 | PCBA |
| 4210 | power supply |
| 4220 | input device |
| 4230 | central controller |
| 4232 | clock |
| 4240 | therapy device controller |
| 4250 | one or more protection circuits |
| 4260 | memory |
| 4270 | transducer |
| 4272 | pressure sensor |
| 4274 | flow rate sensor |
| 4276 | motor speed transducer |
| 4280 | data communication interface |
| 4282 | remote external communication network |
| 4284 | local external communication network |
| 4286 | remote external device |
| 4288 | local external device |
| 4290 | output device |
| 4292 | display driver |
| 4294 | display |
| 4300 | algorithms |
| 4310 | pre-processing module |
| 4312 | interface pressure estimation algorithm |
| 4314 | vent flow rate estimation algorithm |
| 4316 | leak flow rate estimation algorithm |
| 4318 | respiratory flow rate estimation algorithm |
| 4320 | therapy engine module |
| 4321 | phase determination algorithm |
| 4322 | waveform determination algorithm |
| 4323 | ventilation determination algorithm |
| 4324 | inspiratory flow limitation determination |
| | algorithm |
| 4325 | hypopnea determination algorithm |
| 4326 | snore determination algorithm |
| 4327 | airway patency determination algorithm |
| 4328 | target ventilation determination algorithm |
| 4329 | therapy parameter determination algorithm |
| 4330 | therapy control module |
| 4340 | one or more methods |
| 5000 | humidifier |
| 6320 | conduit |
| 8000 | system |
| 9000 | RFID system |
| 9100A | first antenna |
| 9100A′ | RFID reader |
| 9100B | second antenna |
| 9100C | third antenna |
| 9100C′ | RFID reader |
| 9105 | electrical connection |
| 9105A | distal portion |
| 9106 | Curves |
| 9107 | Sleeve |
| 9200A | first tag |
| 9200A′ | tag |
| 9200B | second tag |
| 9200B′ | tag |
| 9300 | Transceiver |
| 9300′ | Transceiver |

1. A conduit headgear, comprising:
an opening configured to removably couple with an air circuit;
a first portion extending from the opening to a first proximal end, wherein the first proximal end is configured to couple with a patient interface;
a second portion extending from the opening to a second proximal end, wherein the second proximal end is configured to couple with the patient interface;
a first stretchable concertina portion extending along a length of the first portion;
a second stretchable concertina portion extending along a length of the second portion;
a first antenna located at a region adjacent the opening;
a second antenna spaced apart from the first antenna; and
a conductive electrical connection coupling the first antenna and the second antenna, wherein the electrical connection extends along at least a portion of either the first stretchable concertina portion or the second stretchable concertina portion, and where the electrical connection is configured to accommodate stretching of the first stretchable concertina portion or the second stretchable concertina portion.

2. The conduit headgear of claim 1, wherein the second antenna is located at a region adjacent the first proximal end or the second proximal end.

3. The conduit headgear of claim 1, wherein the electrical connection has a serpentine or a zig-zag path at least where the electrical connection extends along the at least a portion of either the first stretchable concertina portion or the second stretchable concertina portion.

4. The conduit headgear of claim 1, wherein the electrical connection is formed of a wire or a stretchable material.

5. The conduit headgear of claim 1, wherein the electrical connection is located on an external surface of the first portion or the second portion.

6. The conduit headgear of claim 1, wherein the electrical connection is located within a wall of the first portion or the second portion.

7. The conduit headgear of claim 1, wherein the electrical connection is incorporated as part of a sleeve covering an outer surface of at least one of the first portion or the second portion.

8. The conduit headgear of claim 7, wherein the sleeve is formed of a stretchable textile material.

9. The conduit headgear of claim 7, wherein the electrical connection is in the form of a conductive trace, a conductive thread, or a metal gel.

10. The conduit headgear of claim 9, wherein the electrical connection is printed, thermally bonded, or sewn on the sleeve.

11. The conduit headgear of claim 1, wherein the electrical connection is a stretchable conductive silicone or thread printed or thermally bonded to an external surface of the first portion or the second portion.

12. The conduit headgear of claim 1, wherein the first antenna encircles the opening.

13. A conduit headgear, comprising:
an opening configured to removably couple with an air circuit;
a first portion extending from the opening to a first proximal end, wherein the first proximal end is configured to couple with a patient interface;
a second portion extending from the opening to a second proximal end, wherein the second proximal end is configured to couple with the patient interface;
a first stretchable concertina portion extending along a length of the first portion;
a second stretchable concertina portion extending along a length of the second portion;
a first antenna located at a region adjacent the opening;
a second antenna spaced apart from the first antenna; and
a sleeve covering at least one of the first portion or the second portion, wherein the sleeve comprises a conductive electrical connection extending along the sleeve and electrically coupling the first antenna and the second antenna.

14. The conduit headgear of claim 13, wherein the sleeve is formed of a stretchable textile material.

15. The conduit headgear of claim 13, wherein the sleeve is thermally bonded to an outer surface of at least one of the first portion or the second portion.

16. The conduit headgear of claim 13, wherein at least one of the first antenna or the second antenna is incorporated as part of the sleeve.

17. The conduit headgear of claim 13, wherein the sleeve is removably coupled to the first portion or the second portion.

18. The conduit headgear of claim 13, wherein the sleeve covers the first portion and the second portion.

19. The conduit headgear of claim 13, wherein the second antenna is located at a region adjacent the first proximal end or the second proximal end.

20. A conduit headgear, comprising:
an opening configured to removably couple with an air circuit;
a first portion extending from the opening to a first proximal end, wherein the first proximal end is configured to couple with a patient interface;
a second portion extending from the opening to a second proximal end, wherein the second proximal end is configured to couple with the patient interface;
a first stretchable concertina portion extending along a length of the first portion;
a second stretchable concertina portion extending along a length of the second portion;
a first antenna located at a region adjacent the opening;
a second antenna spaced apart from the first antenna; and
a stretchable conductive electrical connection electrically coupling the first antenna and the second antenna, wherein the electrical connection extends along at least a portion of either the first stretchable concertina portion or the second stretchable concertina portion.

21. The conduit headgear of claim 20, wherein the electrical connection is formed of conductive silicone.

22. The conduit headgear of claim 20, wherein the electrical connection is in the form of a thread or wire.

23. The conduit headgear of claim 20, wherein the electrical connection is printed or thermally bonded on an external surface of the first portion or the second portion.

24. The conduit headgear of claim 1, wherein the first antenna is part of an RFID tag.

25. A system comprising:
the conduit headgear of claim 1;
a third antenna that is electrically coupled to a power source; and
at least one hardware processor configured to perform operations comprising:
causing the third antenna to generate a first RF field that supplies energy that is transferred, via the electrical connection, to the second antenna to generate a second RF field.

26. The system of claim 25, wherein the patient interface includes a second RFID tag, wherein the second RF field is used to read data from the second RF field.

27. The system of claim 26, wherein the operations further comprise:
performing a determination as to whether the opening of the conduit headgear has been coupled with the air circuit, wherein the third antenna is caused to generate the first RF field based on the determination that the opening of the conduit headgear has been coupled with the air circuit.

28. A conduit headgear, comprising:
an opening configured to removably couple with an air circuit;
a first portion extending from the opening to a first proximal end, wherein the first proximal end is configured to couple with a patient interface;
a second portion extending from the opening to a second proximal end, wherein the second proximal end is configured to couple with the patient interface;
a first stretchable concertina portion extending along a length of the first portion;
a second stretchable concertina portion extending along a length of the second portion;
a first RFID tag at a region adjacent the opening; and
an RFID reader configured to communicate with a second RFID tag that is different from the first RFID tag;
a stretchable conductive electrical connection electrically coupling the RFID tag and the RFID reader, wherein the electrical connection extends along at least a portion of either the first stretchable concertina portion or the second stretchable concertina portion,
wherein the first RFID tag is configured to wirelessly receive power, and provide power, via the stretchable conductive electrical connection, to the RFID reader thereby power the RFID reader to communicate with the second RFID tag.
